(19) United States
(12) Patent Application Publication
(10) Pub. No.: US 2007/0149456 A1
(43) Pub. Date: Jun. 28, 2007

(54) Epithelial sodium channel inhibiting agents and uses therefor

(75) Inventors: James B. Bruns, Imperial, PA (US); Marcelo D. Carattino, Pittsburgh, PA (US); Rebecca P. Hughey, McDonald, PA (US); Thomas R. Kleyman, Pittsburgh, PA (US); Shaohu Sheng, Cheswick, PA (US)

Correspondence Address: JESSE A. HIRSHMAN, ESQ., 1722 MURRAY AVENUE, PITTSBURGH, PA, 15217, US

(73) Assignee: University of Pittsburgh, Pittsburgh, PA (US)

(21) Appl. No.: 11/637,568

(22) Filed: Dec. 12, 2006

Related U.S. Application Data
(60) Provisional application No. 60/749,738, filed on Dec. 13, 2005.
(60) Provisional application No. 60/754,801, filed on Dec. 29, 2005.

(51) Int. Cl. A61K 38/17; C07K 14/47

(52) U.S. Cl. 514/12; 530/324

(57) ABSTRACT

Disclosed herein are peptide cleavage products, generated during natural processing of epithelial sodium channel α- and γ-subunits and their use in inhibiting epithelial sodium channel activity. Also disclosed herein are polypeptide and polypeptide analog derivatives of those agents. These epithelial sodium channel-inhibitory agents may be formulated into a drug product for, without limitation, inhibition of epithelial sodium channel function in a patient's airway, useful in treating, among other diseases or conditions: hypertension, congestive heart failure, cirrhosis, nephrotic syndrome, hypokalemia, cystic fibrosis, chronic pulmonary obstructive diseases, such as chronic bronchitis, asthma and bronchiectasis. A method of testing the activity of an epithelial sodium channel-inhibitory agent also is provided.

### CROSS REFERENCE TO RELATED APPLICATIONS

This application claims the benefit under 35 U.S.C. §119(e) to U.S. Provisional Patent Application Nos. 60/749,738, filed Dec. 13, 2005, and 60/754,801, filed Dec. 29, 2005, both of which are incorporated herein by reference in their entirety.

### STATEMENT REGARDING FEDERAL FUNDING

This invention was made with government support under Grant No. R01-DK065161, awarded by the National Institutes of Health. The government has certain rights in this invention.

Inhibitors of epithelial sodium channel function are provided along with methods of use, including treatment of hypertension, congestive heart failure, cirrhosis, nephrotic syndrome, hypokalemia, cystic fibrosis, chronic pulmonary obstructive diseases, such as chronic bronchitis, asthma and bronchiectasis, and methods of identifying useful derivatives of the inhibitors.

The epithelial sodium channel (ENaC) mediates Na⁺ entry across the apical membrane of the distal nephron, airway and alveoli, and distal colon. ENaC has a key role in regulating the volume of airway surface liquids. Over-expression of β-ENaC in transgenic mice produces a phenotype similar to cystic fibrosis with airway surface liquid volume depletion, mucus obstruction, goblet cell metaplasia, neutrophil inflammation and poor bacterial clearance (Mall, M., Grubb, B. R., Harkema, J. R., O'Neal, W. K., and Boucher, R. C. 2004. Increased airway epithelial Na⁺ absorption produces cystic fibrosis-like lung disease in mice. Nat.Med. 10:487-493). It has been proposed that increased activity of ENaC in the airways of patients with cystic fibrosis contributes to the pathogenesis of this disease, and that inhibition of airway ENaC activity may be of therapeutic benefit. While the diuretic amiloride is an effective ENaC inhibitor and is used clinically as a potassium-sparing diuretic, it is rapidly cleared from the airway and is an ineffective inhibitor of airway Na⁺ channels. Unfortunately, effective long-acting blockers of airway Na⁺ channels are not available.

### SUMMARY

Provided are epithelial sodium channel-inhibitory agents, compositions that contain the agents and methods of inhibiting epithelial sodium channel activity. Also provided are methods of characterizing epithelial sodium channel-inhibitory agents. The epithelial sodium channel-inhibitory agents are polypeptides, polypeptide analogs or modified versions thereof corresponding to a 26-mer furin-cleaved fragment of the epithelial sodium channel α-subunit and a 43-mer furin/CAP-1-cleaved fragment of the epithelial sodium channel γ-subunit. These agents are useful in treating any disease or condition that would benefit from inhibition of epithelial sodium channel activity, including, without limitation: hypertension, congestive heart failure, cirrhosis, nephrotic syndrome, hypokalemia, cystic fibrosis and chronic pulmonary obstructive diseases, such as chronic bronchitis, asthma and bronchiectasis.

In one embodiment, epithelial sodium channel-inhibitory agent is provided comprising a polypeptide or polypeptide analog, other than a full length epithelial sodium channel γ-subunit, comprising the sequence:
X₁X₂X₃SX₄X₅X₆X₇X₈X₉GX₁₀X₁₁PX₁₂FX₁₃X₁₄X₁₅X₁₆PLX₁₇X₁₈FX₁₉X₂₀X₂₁X₂₂X₂₃X₂₄X₂₅ARDFX₂₆X₂₇X₂₈X₃₀X₂₉KRK(Formula II)(SEQ ID NO: 1)
wherein X₁₋₃₀ are, independently, any amino acid, X₃₀ may or may not be present and any 0, 1, 2 or 3 amino acids listed specifically in Formula II may be substituted, or conservatively substituted, with another amino acid.

In another embodiment, an epithelial sodium channel-inhibitory agent other than a full length epithelial sodium channel α-subunit is provided, the agent comprising one or more polypeptide or polypeptide analog comprising a sequence: DX₁X₂X₃X₄X₅PHPLQRLX₆X₇PX₈PX₉X₁₀X₁₁X₁₂RX₁₃X₁₄R (SEQ ID NO: 2); DLRGX₄LPHPLQRLRVPPPPX₁₀X₁₁ARX₁₃AR (SEQ ID NO: 2, wherein 2=L, 3=R, 4=G, 6=L, 14=R, 15=V, 17=P, 19=P, 22=A, and 25=A); PHPLQRL (SEQ ID NO: 2, residues 7-13); PHPLQRLX₆X₇PX₈P (SEQ ID NO: 2, residues 7-18); LPHPLQRL (SEQ ID NO: 2, residues 6-13); PHPLQRLX₆X₇PX₈P (SEQ ID NO: 2, residues 7-18); DX₁X₂X₃X₄X₅PHPLQRLX₆X₇PX₈P (SEQ ID NO: 2, residues 1-18); PHPLQRLX₆X₇PX₈PX₉X₁₀X₁₁X₁₂ (SEQ ID NO: 2, residues 7-22); or PHPLQRLX₆X₇PX₈PX₉X₁₀X₁₁X₁₂RX₁₃X₁₄R (SEQ ID NO: 2, residues 7-26), wherein X₁₋₁₄ are, independently, any amino acid and any 0, 1, 2 or 3 amino acids listed specifically in the sequences are substituted with another amino acid, wherein the agent is not a polypeptide consisting of the sequence DLRGALPHPLQRLRTPPPPNPARSAR (SEQ ID NO: 4, residues 206-231).

In a further embodiment, a composition is provided comprising an epithelial sodium channel-inhibitory agent, the agent comprising an amount of the agent effective to inhibit epithelial sodium channel activity in a patient, wherein the agent is a polypeptide or polypeptide analog comprising the sequence:
X₁X₂X₃SX₄X₅X₆X₇X₈X₉GX₁₀X₁₁PX₁₂FX₁₃X₁₄X₁₅X₁₆PLX₁₇X₁₈FX₁₉X₂₀X21X₂₂X₂₃X₂₄X₂₅ARDFX₂₆X₂₇X₂₈X₃₀X₂₉KRK(Formula II) (SEQ ID NO: 1)
wherein X₁₋₃₀ are, independently, any amino acid, X₃₀ may or may not be present and any 0, 1, 2 or 3 amino acids listed specifically in Formula II are substituted with another amino acid, and a pharmaceutically acceptable excipient. A method of inhibiting epithelial sodium channel activity in cells of a patient's airway also is provided, comprising administering to the patient an amount of the composition effective to inhibit activity of epithelial sodium channel in cells of the patient's airway, thereby inhibiting activity of the epithelial sodium channel in the patient's airway.

In another embodiment, a composition is provided comprising an epithelial sodium channel-inhibitory agent, the agent comprising an amount of the agent effective to inhibit epithelial sodium channel activity in a patient, wherein the agent is one or more polypeptide or polypeptide analog comprising a sequence:

DX₁X₂X₃X₄X₅PHPLQRLX₆X₇PX₈PX₉X₁₀X₁₁X₁₂RX₁₃X₁₄R (SEQ ID NO: 2); DLRGX₄LPHPLQRLRVPPPPX₁₀X₁₁ARX₁₃AR (SEQ ID NO: 2, wherein residues 2=L, 3=R, 4=G, 6=L, 14=R, 15=V, 17=P, 19=P, 22=A, and 25=A); PHPLQRL (SEQ ID NO: 2, 7-13); LPHPLQRL (SEQ ID NO: 2, residues 6-13); PHPLQRLX₆X₇PX₈P (SEQ ID NO: 2, residues 7-18); DX₁X₂X₃X₄X₅PHPLQRLX₆X₇PX₈P (SEQ ID NO: 2, residues 1-18); PHPLQRLX₆X₇PX₈PX₉X₁₀X₁₁X₁₂ (SEQ ID NO: 2, residues 7-22); or PHPLQRLX₆X₇PX₈PX₉X₁₀X₁₁X₁₂RX₁₃X₁₄R (SEQ ID NO: 2, residues 7-26), wherein X₁₋₁₄ are, independently, any amino acid and any 0, 1, 2 or 3 amino acids listed specifically in the sequences are substituted with another amino acid, and a pharmaceutically acceptable excipient. A method of inhibiting epithelial sodium channel activity in cells of a patient's airway is provided, comprising administering to the patient an amount of the composition effective to inhibit activity of epithelial sodium channel in cells of the patient's airway, thereby inhibiting activity of the epithelial sodium channel in the patient's airway.

In a further embodiment, a method of characterizing the activity of an epithelial sodium channel-inhibitory agent is provided, comprising testing the epithelial sodium channel-inhibitory agent for inhibition of epithelial sodium channel activity, the agent comprising the sequence:

DX₁X₂X₃X₄X₅PHPLQRLX₆X₇PX₈PX₉X₁₀X₁₁X₁₂RX₁₃X₁₄R (SEQ ID NO: 2); DLRGX₄LPHPLQRLRVPPPPX₁₀X₁₁ARX₁₃AR (SEQ ID NO: 2, wherein residues 2=L, 3=R, 4=G, 6=L, 14=R, 15=V, 17=P, 19=P, 22=A, and 25=A); PHPLQRL (SEQ ID NO: 2, residues 7-13); LPHPLQRL (SEQ ID NO: 2, residues 6-13); PHPLQRLX₆X₇PX₈P (SEQ ID NO: 2, residues 7-18); DX₁X₂X₃X₄X₅PHPLQRLX₆X₇PX₈P (SEQ ID NO: 2, residues 1-18); PHPLQRLX₆X₇PX₈PX₉X₁₀X₁₁X₁₂ (SEQ ID NO: 2, residues 7-22); or PHPLQRLX₆X₇PX₈PX₉X₁₀X₁₁X₁₂RX₁₃X₁₄R (SEQ ID NO: 2, residues 7-26), wherein X₁₋₁₄ are, independently, any amino acid and any 0, 1, 2 or 3 amino acids listed specifically in the sequences are substituted with another amino acid. Alternately, the agent comprises the sequence:
X₁X₂X₃SX₄X₅X₆X₇X₈X₉GX₁₀X₁₁PX₁₂FX₁₃X₁₄X₁₅X₁₆PLX₁₇X₁₈FX₁₉X₂₀X₂₁X₂₂X₂₃X₂₄X₂₅ARDFX₂₆X₂₇X₂₈X₃₀X₂₉KRK(Formula II) (SEQ ID NO: 1)
wherein X₁₋₃₀ are, independently, any amino acid, X₃₀ may or may not be present and any 0, 1, 2 or 3 amino acids listed specifically in Formula II are substituted with another amino acid.

### BRIEF DESCRIPTION OF THE DRAWINGS

FIGS. 1A-1C show lack of furin-dependent proteolysis effectively reduces the open probability of channels with a degenerin site mutation that favors a high open probability. Experiments were performed 20-24 h after injection of oocytes with cRNAs for wild-type or mutant ENaCs. The α and γ subunits had amino- (HA) and carboxyl- (V5) terminal epitope tags. FIG. 1A, Mutation of furin cleavage sites in both the α and γ subunits reduced the activity of channels with a degenerin site mutation (βS518K). Whole cell ENaC currents were measured at −60 mV in oocytes expressing either αβγ, αRtripleAβγR143A, αβS518Kγ or αRtripleAβS518KγR143A. The mutation αRtripleA blocks both furin-cleavage sites in α. Significant differences were observed between αβγ controls versus αRtripleAβγR143A or αβS518Kγ (grey bars, p<0.001, n=24-26, Kruskal Wallis test (nonparametric ANOVA) followed by Dunn's multiple comparisons post-test). FIGS. 1B and 1C, Mutations of furin consensus cleavage sites in the α and γ subunits affect the gating of ENaCs with a degenerin site mutation. Single channel tracings were obtained in the cell-attached mode as described under “Experimental procedures.” The closed state is indicated by “C”. Recordings were performed at an applied pipette potential of +60 mV. Upper tracings show representative recordings of αβS518Kγ (n=11) (B) or αRtripleAβS518KγR143A (n=5) (C) channels. Normalized amplitude histograms are presented at the right side of each recording.

FIGS. 2A-2C show the tract Asp206-Arg231 in the α-subunit inhibits channel activity. TEV was performed in oocytes expressing either wild-type or mutant ENaCs. The αsubunit had amino- (HA) and carboxyl- (V5) terminal epitope tags. FIG. 2A, Cleavage at both furin consensus sites within the α subunit of ENaC is required for expression of active channels. ENaC currents were recorded before and 5 min following treatment with 2 μg/ml trypsin (close bars). Experiments were performed with a solution containing (in mM) 100 Na⁺ gluconate, 1.54 CaCl₂, 5 BaCl₂, 10 TEA, 10 Hepes, pH 7.4. A gray bar indicates statistically significant differences in amiloride-sensitive currents between αβγ control versus αR205Aβγ, αR231Aβγ, or αRtripleAβγ (p<0.001, Kruskal Wallis test (nonparametric ANOVA) followed by Dunn's multiple comparisons post-test). Amiloride-sensitive currents following treatment with trypsin were not significantly different between the 4 groups (black bars). Experiments were performed with 15-18 oocytes for each group. FIG. 2B, ENaCs lacking the tract αAsp206-Arg231 are active in the presence or absence of furin cleavage. Whole cell currents in oocytes expressing αΔ206-231βγ and αR205A,Δ206-231βγ were similar to or greater than currents measured in oocytes expressing wild-type αβγ. Whole cell currents were statistically different between αβγ control versus αRtripleAβγ (p<0.01) and αR205A,Δ206-231βγ (p<0.05, Kruskal Wallis test (nonparametric ANOVA) followed by Dunn's multiple comparisons post-test). Experiments were performed with 15 oocytes for each group. FIG. 2C, Characterization of α-subunit processing by furin. ENaC was immunoprecipitated with anti-V5 antibodies from extracts of MDCK cells transiently expressing GFP control, wild-type αβγ, αΔ206-231βγ, αR205A,Δ206-231βγ or αRtripleAβγ and immunoblotted for the V5-tagged α subunit in each case. Numbers to the left of the gel represent mobility of molecular weight markers.

FIGS. 3A-3F show ENaC currents are blocked by the synthetic peptide α-26 representing the tract released from α by furin cleavage. Experiments were performed in oocytes expressing either wild-type or mutant ENaCs. I/I₀ represents the ratio of the amiloride-sensitive current before and after 3 min of perfusion with the peptide α-26. FIG. 3A, Concentration-response for α-26 in oocytes expressing wild-type ENaC (n=9-14). FIG. 3B, effect of related peptides (10 μM) on ENaC currents. Control, no peptide; α-26; SCR, α-26 with scrambled amino acid sequence; P/A, α-26 with all Pro substituted with Ala; R/E, α-26 with all Arg substituted with Glu. Control versus either α-26 (p<0.001), P/A peptide (p<0.05), or R/E peptide (p<0.01) (n=12-15, Kruskal-Wallis test (Nonparametric ANOVA) following by Dunn's multiple comparisons test). FIG. 3C, time course of inhibition of amiloride-sensitive currents by α-26 added at time zero (n=14). FIG. 3D, the block by α-26 is reversible. ENaC currents were determined before (basal), during treatment (T) and following washout (W/O) in oocytes expressing ENaC and treated with α-26 (1 μM) (open circles) or vehicle (closed circles) (n=12-13). FIG. 3E, α-26 and amiloride bind at different sites. Amiloride concentration-response in the presence (open circles) or absence (closed circles) of α-26 (2.5 μM) (n=12). FIG. 3F, α-26 (1 μM) does not block uncleaved channels. The α subunit had amino-(HA) and carboxyl- (V5) terminal epitope tags in each case. αβγ versus αRtripleAβγ or αR205AΔ206-231βγ (p<0.001, n=9-11, Kruskal-Wallis test (Nonparametric ANOVA) following by Dunn's multiple comparisons test).

FIGS. 4A-4C show the synthetic peptide α-26 representing αAsp206-Arg231 reduces ENaC open probability. Experiments were performed in oocytes expressing wild-type ENaC. FIG. 4A—NPo measured during the first 6 min of recording for controls (no peptide) (n=5) or experiments performed with the pipette back filled with α-26 (10 μM) (n=7). Controls versus α-26 (p<0.05, unpaired t test). FIGS. 4B and 4C—representative single channel recordings performed in oocytes expressing ENaC under control conditions (FIG. 4B) or with a pipette back filled with α-26 (10 μM) (FIG. 4C), respectively.

FIGS. 5A-5G show endogenous ENaCs in primary cultures of human airway epithelial cells (HAE) and a mouse kidney cell line (mpkCCD_{c14}) are inhibited by peptide α-26. FIGS. 5A and 5B—concentration-response for α-26 in HAE (FIG. 5A) and mpkCCD_{c14} cells (FIG. 5B). I/I₀ is the ratio of the amiloride-sensitive current before and 5 min following the addition of peptide. FIGS. 5C and 5D—representative recordings of the effect of α-26 (gray) or SCR (black) on HAE (FIG. 5C) and mpkCCD_{c14} (FIG. 5D) monolayers. Arrows indicate the addition of peptides at a concentration of 2 μM and 50 μM, and amiloride (amil) at 10 μM. FIGS. 5E and 5F—responses of HAE (FIG. 5E) and mpkCCD_{c14} (FIG. 5F) monolayers to α-26, SCR, or control (no peptide). I/I₀ estimated under basal conditions (open bars) and following the addition of peptide at 2 μM (closed bars) and 50 μM (gray bars) are plotted (n=6-8). FIG. 5G—amiloride-sensitive currents were recorded in oocytes expressing wild-type ENaC with or without prostasin, before and after 3 min of perfusion with α-26. Significant differences in the current response to α-26 between oocytes expressing wild-type ENaC and co-expressing ENaC and prostasin were observed at 1 μM (p<0.001, n=10-11) and 10 μM (p<0.05, n=7) (Kruskal-Wallis test (Nonparametric ANOVA) following by Dunn's multiple comparisons test).

FIGS. 6A and 6B are graphs showing the γ-peptide reversibly inhibits endogenous ENaC in a mouse cortical collecting duct (mCCD_{c14}) cell line (6A—reversibility and 6B—dose response).

FIG. 7 is a graph showing the γ-peptide is a potent inhibitor of ENaC mediated transepithelial sodium transport in HAE cells.

FIG. 8 provides the amino acid sequence of the human epithelial sodium channel α-subunit (SEQ ID NO: 3).

FIG. 9 provides the amino acid sequence of the mouse epithelial sodium channel α-subunit (SEQ ID NO: 4).

FIG. 10 provides the amino acid sequence of the human epithelial sodium channel γ-subunit (SEQ ID NO: 5).

FIG. 11 provides the amino acid sequence of the mouse epithelial sodium channel γ-subunit (SEQ ID NO: 6).

FIG. 12 provides illustrations of the furin cleavage of the α-peptide (HA-α-V5) and furin/CAP-1 cleavage of the γ-peptide (HA-γ-V5).

FIGS. 13A and 13B show partial alignment of the mouse ENaC α and γ subunits. (FIG. 13A) Identical residues are highlighted in light gray with black lettering while homologous residues are highlighted in dark gray with white lettering. Furin cleavage sites are highlighted in black and a tetra-basic tract in the γ subunit is underlined and bolded. (FIG. 13B) Alignment of peptides released from the α and γ subunits by proteolytic cleavage.

FIG. 14A and 14B show prostasin co-expression activates ENaC by inducing cleavage of the γ subunit at RKRK¹⁸⁶ (SEQ ID NO: 7). (FIG. 14A) Extracts of MDCK cells transiently transfected with epitope-tagged ENaC and with (+) or without (−) prostasin were incubated overnight with anti-V5 antibody conjugated to agarose beads. Irnmunoprecipitates were analyzed by immunoblotting for the V5-tagged γ subunit after SDS-PAGE. Note that co-expression of ENaC with prostasin produces a smaller fragment of the γ subunit (see asterisk) that is absent for the RKRK¹⁸⁶ (SEQ ID NO: 7)/QQQQ (SEQ ID NO: 19) mutant (γmut). Numbers to the right of the gel represent the mobility of BioRad Precision Plus protein standards in kDa. Representative immunoblot is shown (n=3). (FIG. 14B) Xenopus oocytes were co-injected with cRNAs encoding mouse αβγ or αβγmut ENaC with (+) or without (−) prostasin. Whole cell amiloride sensitive currents were measured the following day at a clamp potential of −100 mV (n=28-30). *p<0.005 compared to αβγ without prostasin.

FIGS. 15A-15C. Prostasin increases the open probability of ENaC. Single channel recordings were performed in the cell-attached patch configuration using oocytes co-expressing wild type ENaC with or without prostasin. Representative single channel recordings from oocytes expressing wild type channels with (FIG. 15B) and without (FIG. 15A) prostasin. Amplitude histograms are presented at the right side of each recording. “C” indicates the closed state. (FIG. 15C) Open probabilities of ENaC estimated from oocytes co-expressing wild type channels with or without prostasin in the same batch of oocytes (n=7-11, p<0.0001, unpaired Student's t test).

FIGS. 16A and 16B show the tract γGlu144-Lys186 inhibits channel activity. Either wild-type or mutant ENaCs were expressed in oocytes and MDCK cells. The γ subunit had amino-terminal HA and carboxyl-terminal V5 epitope tags. (FIG. 16A) Deletion of the tract γGlu144-Lys186 between both the furin and prostasin cleavage sites increases ENaC activity. Amiloride sensitive whole cell currents were significantly different between oocytes expressing αβγ versus αβγ R143A,RKRK (SEQ ID NO: 7)/Q4 (γR/A, RKRK (SEQ ID NO: 7)/Q4) (p<0.05) or αβγ R143A,Δ144-186 (γR/AΔ) channels (p<0.001, Kruskal Wallis test (nonparametric ANOVA) followed by Dunn's multiple comparisons post-test, n=16). (FIG. 16B) Characterization of γ-subunit processing in MDCK cells. ENaC was immunoprecipitated with anti-V5 antibodies from extracts of MDCK cells transiently expressing wild-type αβγ or αβγ R143A,Δ144-186. Following SDS-PAGE, the immunoprecipitates were blotted for the V5-tagged γ subunit. Numbers to the right of the gel represent the mobility of BioRad Precision Plus protein standards in kDa. Representative immunoblot is shown (n=3).

FIGS. 17A-17C show deletion of the tract γGlu144-Lys186 increases ENaC single channel open probability. Single channel recordings were performed in the cell-attached configuration of patch-clamp from oocytes expressing wild type αβγ or αβγR143A,Δ144-186. Representative single channel recordings of wild type αβγ (FIG. 17A) or αβγR143A,Δ144-186 (FIG. 17B) channels. Amplitude histograms are presented at the right side of each recording. “C” indicates the closed state. (FIG. 17C) Open probabilities were statistically different in oocytes expressing wild type αβγ and αβγR143A,Δ144-186 channels (p<0.005, unpaired Student's t test with Welch correction, n=4).

FIGS. 18A-18C show a peptide (γ-43) representing the γ cleavage product inhibits ENaC-mediated transepithelial Na⁺ transport in endogenously expressing epithelia. (FIG. 18A) Mouse mpkCCD_{c14} cells cultured on permeable membrane supports and mounted in a modified Ussing chamber were continuously short-circuited. Transepithelial resistance was monitored by applying a 4 mV bipolar pulse every 60 seconds. Where indicated, a 43-residue peptide corresponding to the tract Glu144-Lys186 of mouse γ ENaC (γ-43) was added to the apical chamber at a final concentration of 3 μM. This led to a marked inhibition of transepithelial Na⁺ transport that was relieved by washout of the peptide. Subsequent application of 20 μM amiloride to the apical chamber confirmed that the short circuit current reflected ENaC-mediated transepithelial Na⁺ transport. Tracing is representative of 5 experiments. mpkCCD_{c14} cells (FIG. 18B) or HAE cells (FIG. 18C) cultured and voltage clamped as above were exposed to increasing concentrations of either γ-43 (closed circles) or a scrambled peptide (open circles). While the scrambled peptide failed to inhibit amiloride sensitive short circuit currents, the γ cleavage product inhibited in a dose dependent manner (n=8-9).

FIGS. 19A and 19B show apparent competition between amiloride and the γ-43 peptide for ENaC block. mpkCCDc14 cells cultured on permeable membrane supports and mounted in a modified Ussing chamber were continuously short-circuited by a voltage clamp amplifier (Physiologic Instruments, San Diego, Calif.). (FIG. 19A) Dose response curves for amiloride in the presence (open circles) or absence (closed circles) of 3 μM γ-43 peptide. (FIG. 19B) Amiloride IC50 values in the absence or presence of 3 μM γ-43 peptide (n=18). *p<0.001

### DETAILED DESCRIPTION

The use of numerical values in the various ranges specified in this application, unless expressly indicated otherwise, are stated as approximations as though the minimum and maximum values within the stated ranges are both preceded by the word “about”, whether or not the term “about” is present. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases.

As used herein, and unless indicated otherwise, “a” and/or “an” refer to one or more.

As used herein, the term “comprising”, is intended to be inclusive and/or open-ended and does not exclude additional, unrecited elements or method steps. The transitional phrase “consisting of” excludes any element, step, or ingredient not specified in the claim.

ENaC is likely a tetramer (α₂βγ) composed of three homologous subunits with each containing two transmembrane domains connected by a large extracellular loop, and cytoplasmic amino- and carboxyl-termini. Residues preceding and within the second membrane-spanning domain constitute the channel pore. A number of factors have been identified that modulate ENaC activity, including proteolysis. Proteolytic cleavage of ENaC subunits activates the channel by increasing channel open probability. ENaC activity is stimulated by treatment with extracellular trypsin, or by co-expression of ENaC and specific serine proteases, referred to as channel activating proteases (or CAPs) (Vallet, V., Chraïbi, A., Gaeggeler, H.-P., Horisberger, J.-D., and Rossier, B. C. 1997. An epithelial serine protease activates the amiloride-sensitive sodium channel. Nature 389:607-610). ENaC activity in human bronchial epithelial (HBE) cells was blocked by serine protease inhibitors such as aprotinin or bikunin, which inhibit prostasin, also referred to as the well-characterized CAP-1, as well as other serine proteases. Furin, a ubiquitous serine protease not blocked by aprotinin that resides primarily in the trans-Golgi network, both cleaves and activates ENaC (Hughey, R. P., Bruns, J. B., Kinlough, C. L., Harkleroad, K. L., Tong, Q., Carattino, M. D., Johnson, J. P., Stockand, J. D., and Kleyman, T. R. 2004. Epithelial sodium channels are activated by furin-dependent proteolysis. J.Biol. Chem. 279:18111-18114). Using epitope-tagged subunits and site-specific mutagenesis of minimal furin cleavage consensus sites (RXXR (SEQ ID NO: 2, residues 23-36), where X is any residue and cleavage occurs following the distal Arg (R)), we observed that α-ENaC was cleaved twice (after R205 and R231) while γ-ENaC was cleaved once (after R143) in a furin-dependent manner, consistent with proteolytic processing of ENaC in the biosynthetic pathway en route to the cell surface (Hughey, R. P. et al. 2004. J. Biol. Chem. 279:18111-18114). Furin mediated proteolysis of α-ENaC is blocked by mutations R205A and R2312A in α-ENaC, and furin-mediated proteolysis of γ-ENaC is blocked an R143A mutation in γ-ENaC. Furin dependent processing of ENaC was also required for channel activation in mouse cortical collecting duct cells (mpkCCD_{c14}) and transfected Chinese Hamster Ovary (CHO) and Madin-Darby canine kidney (MDCK) cells.

α-ENaC must be cleaved at both furin consensus sites to activate the channel (Sheng, S., Carattino, M. D., Bruns, J. B., Hughey, R. P., and Kleyman, T. R. 2006. Furin cleavage activates the epithelial Na⁺ channels by relieving Na⁺ self-inhibition. Am J Physiol Renal Physiol. 290:F1488-96). Proteolytic cleavage at the two furin sites releases a 26-mer peptide, as illustrated in FIG. 12 (HA-α-V5). As shown herein, this 26-mer peptide affects ENaC activity. Perfusion of Xenopus oocytes expressing mouse ENaC with a synthetic 26-mer peptide (α-26) representing the sequence released by furin led to a dose-dependent inhibition of channel activity (Ki of ˜3 μM). A control (scrambled) peptide did not block channel activity. Endogenous ENaC activity in HBE or mpkCCD_{c14} cells is blocked by the peptide with a Ki of ˜50-100 μM. The α-peptide (α-26) is rich in Pro and basic amino acid residues. Mutation of all Pro or basic residues led to a loss of inhibitory activity.

It was previously observed that γ-ENaC is cleaved at the single furin consensus site (Hughey, R. P. et al. 2004. J. Biol. Chem. 279:18111-18114). We have observed that a second CAP-1/prostasin-dependent cleavage event occurs within the sequence RKRK¹⁸⁶ (SEQ ID NO: 7), releasing a 43-mer peptide, as illustrated in FIG. 12 (HA-γ-V5). Mutation of the CAP-1/prostasin cleavage site (R183Q/K184Q/R185Q/K186Q) blocks CAP-1/prostasin-dependent cleavage and activation of channel activity. A synthetic 43-mer γ-peptide (γ-43), corresponding to the peptide released by furin and CAP-1 cleavage, inhibited endogenous ENaC activity in HBE and mpkCCD_{c14} cells with an apparent Ki of ˜2 μM. A control (scrambled) peptide did not block channel activity. In summary, peptides released from ENaC as a result of proteolytic processing of the channel can inhibit ENaC activity when added back to the active channel. These novel peptide inhibitors of ENaC should serve as the basis for the development of novel drug products useful in treating diseases or conditions that would benefit from inhibition of epithelial sodium channel activity, including, without limitation: chronic obstructive pulmonary disease; cystic fibrosis; chronic or acute bronchitis, bronchiolitis, pharyngitis, laryngitis; or any airway infection.

Novel inhibitors of epithelial sodium channel function therefore are described herein. The inhibitors are polypeptide fragments generated upon proteolytic activation of the epithelial sodium channel α and γ subunits and analogs thereof. In the case of the epithelial sodium channel α subunit, the polypeptide typically is a 26-mer generated by furin cleavage. In the case of the epithelial sodium channel γ subunit, the polypeptide is a 43-mer generated by the action of both furin and channel activating protease 1 (CAP-1, also known as prostasin). As shown herein, these polypeptides inhibit epithelial sodium channel activity when applied to cells expressing epithelial sodium channels. As such, like amiloride, these polypeptides are useful in treating any disease or condition in which inhibition of the epithelial sodium channel activity is desirable, such as hypertension, congestive heart failure, cirrhosis, nephrotic syndrome or hypokalemia, but unlike amiloride, these polypeptides are not expected to be readily cleared from the airways, making them useful in treating cystic fibrosis, chronic pulmonary obstructive diseases, such as chronic bronchitis, asthma and bronchiectasis, or any other disease or condition in which increased airway secretion is desired, as in the clearance of infectious agents or particular matter in the case of inhalation of excessive particulate matter.

Polypeptides cleaved from the α and γ subunits of an epithelial sodium channel during activation (processing) of the epithelial sodium channel, along with derivatives and analogs thereof, are provided herein. These cleaved polypeptides are shown to inhibit activity of the human epithelial sodium channel, non-limiting examples of which are provided in Tables A (α subunit) and C (γ subunit), below. Derivatives of the cleaved, native polypeptides are expected to inhibit activity of the human epithelial sodium channel as well. As such, the native polypeptide cleavage products as well as derivatives thereof are referred to herein as “epithelial sodium channel-inhibitory agent(s)” or “epithelial sodium channel-inhibitory polypeptide(s).” As used herein, the “agents” include as a class, polypeptides, but also includes polypeptide analogs and modified polypeptides as are known in the art.

Modified polypeptides are, in many instances, more stable than un-modified polypeptides because the modified residue(s) render the polypeptide resistant to protease cleavage/degradation. As such, by modification of the polypeptide backbone, including, without limitation, derivatization of one or more amino acids or attachment of chemical groups onto a polypeptide chain, the usefulness of a polypeptide as a drug may be increased—the modification increasing drug parameters, such as bioavailability and half-life. A large number of potential modifications are listed, for example and without limitation, in U.S. Pat. No. 6,075,121, including use of α and β ester linkages, thioamines and N-hydroxylation. However, two polypeptide analog structures may prove to be most useful in improving bioavailability and/or half-life of the polypeptides described herein, including circularization and peptoid modification.

A polypeptide may be circularized by any method, such as, without limitation, using a safety catch liner as described by Borne and co-workers (Borne, GT et al. 2005. A convenient method for synthesis of cyclic peptide libraries Methods Mol Biol. 298:151-65). By circularization, the 3D structure of the polypeptide may be altered, and the addition of a small spacer sequence, such as 1-25 flexible glycine residues prior to peptide ligation (circularization), is expected to be helpful in retaining polypeptide function.

A second, well-characterized polypeptide modification that shows promise in producing polypeptides that retain function of the “parent” polypeptide, yet show better protease resistance is the use of “peptoids.” A peptoid is a polypeptide containing one or more N-substituted glycine residues. An N-substituted amino acid residue has a standard amino acid side-chain pendant from the N, rather than from the α carbon. For example NVal has a 2-propyl group pendant from its N. N-alkyl glycine residues are common peptoid building blocks, which may mimic standard amino acids, such as Val (2-propyl), Leu (isobutyl) or Ile (2-butyl) among the 19 other standard R-group-containing amino acids, or which may contain virtually any R-group, for example any lower alkyl (C-₁₋₆) group, alcohol, amine, organic acid (carboxyl-containing), etc. group. By including N-modified glycine residues, the peptoid is made resistant to proteolysis and may be functionalized in a manner that increases the peptide's bioavailability or tissue localization. U.S. Pat. No. 6,075,121 describes peptoid structures and methods of producing peptoids. U.S. Pat. No. 6,887,845 describes N-palmitoyl derivatized surfactant protein-B. The palmitoyl moieties are thought to interact with cellular lipid bilayers, thereby affecting the bioavailability of the polypeptide.

Also provided are compositions containing an amount of one or more of the described epithelial sodium channel-inhibitory agents effective for use in treating a disease or condition that would benefit from inhibition of epithelial sodium channel activity, including, without limitation: airway conditions such as such as cystic fibrosis, chronic obstructive pulmonary diseases, such as, without limitation, chronic bronchitis, asthma and bronchiectasis; cardiovasculature conditions, as a potassium-conserving diuretic to treat conditions including hypertension, congestive heart failure, cirrhosis, nephrotic syndrome and hypokalemia. Because small molecule drugs, such as amiloride clear from the lungs too rapidly to be therapeutically effective, the epithelial sodium channel-inhibitory agents described herein are thought to be preferred because they will likely clear from the airways much more slowly than the small molecule drug compounds.

Table A provides sequences of the epithelial sodium channel-inhibitory agent cleaved from the α subunit of the epithelial sodium channel of the listed species during activation of the epithelial sodium channel. Table B identifies commonalities between the α-ENaC sequences listed in Table A and provides conservative substitutions for those non-conserved amino acids using one common substitution matrix (BLOSUM62).

TABLE A
Alpha EnaC subunit Cleavage Products

| | |
|---|---|
| Mouse | |
| DLRGALPHPLQRLRTPPPPNPARSAR²³¹ | (SEQ ID NO: 4, res- |
| | idues 206-231) |
| Human | |
| DLRGTLPHPLQRLRVPPPPHGARRAR²³¹ | (SEQ ID NO: 3, res- |
| | idues 179-204) |
| Rat | |
| DLLGAFPHPLQRLRTPPPPYSGRTAR²⁰⁴ | (SEQ ID NO: 8) |
| Bovine | |
| DLREPLPHPLQRLPVPAPPHAARGVR¹⁸⁴ | (SEQ ID NO: 9) |
| Rabbit | |
| DVHPPLPHPLQRLRVPPPRLEARRAR¹⁸³ | (SEQ ID NO: 10) |
| Consensus | |
| DLRGXLPHPLQRLRVPPPPXXARXAR | (SEQ ID NO: 2, |

wherein wherein residues 2 = L, 3 = R, 4 = G,
6 = L, 14 = R, 15 = V, 17 = P, 19 = P, 22 = A,
and 25 = A)

TABLE B
Alpha ENaC subunit Consensus:
DX₁X₂X₃X₄X₅PHPLQRLX₆X₇PX₈PX₉X₁₀X₁₁X₁₂RX₁₃X₁₄R (SEQ ID NO: 2)

| | Residue | Endogenously | Blosum62 | Blosum62 |
|---|---|---|---|---|
| | in Mouse | Present | Substitution | Substitution |
| Residue | or Human | Residues | (score ≧ 0)^{a} | (score > 0)^{a} |
| X₁ | L | L, V | L, V, M, I, F | L, V, M, I |
| X₂ | R | R, L, H | R, L, H, N, E, Q, K, M, I, V, F, T, D, Y | R, L, H, Q, K, M, I, V, N, Y |
| X₃ | G | G, E, P | G, E, P, S, T, A, V, N, D, Q, H, R, K | G, E, P, T, D, Q, K |
| X₄ | A, T | A, T, P | A, T, P, C, S, G, N, D, E, Q, H, K, | A, P, S, G, D |
| X₅ | L | L, F | L, F, M, I, V, Y, W | L, F, M, I, V, Y, W |
| X₆ | R | R, P | R, P, N, E, Q, H, K, T | R, P, Q, K, T |
| X₇ | T, V | T, V | T, V, S, P, G, N, D, E, Q, H, K, A, M, I, L, | T, V, S, P, G, D, M, I, L |
| X₈ | P | P, A | P, A, T, C, S, G, | P, A, T, S |
| X₉ | P | P, R | P, R, T, N, E, Q, H, K | P, R, T, Q, K |
| | | | N, H, Y, L, S, G, D, E, Q, R, K, T, F, Y, W, M, I, | N, H, Y, L, S, D, F, W, M, I, |
| X₁₀ | N, H | N, H, Y, L | V | V |
| X₁₁ | P, G | P, G, S, A, E | P, G, S, A, E, T, V, N, D, Q, K, C, H, R | P, G, S, A, E, T, N, D, Q, K |
| X₁₂ | A | A, G | A, G, C, S, T, V | A, G, S, T |
| X₁₃ | S, R, | S, R, T, G | S, R, T, G, A, N, D, E, Q, K, H, P, V | S, R, T, G, A, N, Q, K, P, D |
| X₁₄ | A | A, V | A, V, C, S, G, M, I, L | A, V, S, M, I, L |

^{a}Includes “Endogenously Present Residues.”

In one embodiment corresponding to the mouse-human examples shown below (mouse polypeptide affecting human epithelial sodium channel), the epithelial sodium channel-inhibitory agent is a polypeptide cleaved from an α-ENac subunit or a derivative or analog thereof (α-ENaC-derived epithelial sodium channel-inhibitory agent), comprising the amino acid sequence: DLRGALPHPLQRLRTPPPPNPARSAR (SEQ ID NO: 4, residues 206-231), DLRGTLPHPLQRLRVPPPPHGARRAR (SEQ ID NO: 3, residues 179-204), or DLRGX₄LPHPLQRLRX₇PPPPX₁₀X₁₁ARX₁₃AR (SEQ ID NO: 2, wherein 2=L, 3=R, 4=G, 6=L, 14=R, 17=P, 19=P, 22=A, and 25=A),where X₄is A or T, X₇is T or V, X₁₀is N or H, X₁₁ is P or G and X₁₃ is S or R. In yet another embodiment, the polypeptide or analog thereof has the mammalian consensus sequence:
DX₁X₂X₃X₄X₅PHPLQRLX₆X₇PX₈PX₉X₁₀X₁₁X₁₂RX₁₃X₁₄R, (Formula I) (SEQ ID NO: 2) or
DLRGX₄LPHPLQRLRVPPPPX₁₀X₁₁ARX₁₃AR(SEQ ID NO: 2, wherein residues 2=L, 3=R, 4=G, 6=L, 14=R, 15=V, 17=P, 19=P, 22=A, and 25=A)
in which one or more of X₁₋₁₄ are independently substituted as shown in Table B under the heading “Endogenously Present” meaning X₁₋₁₄ has values present in known epithelial sodium channel sequences. In a further embodiment, one or more of X₁₋₁₄ are substituted as shown in Table B under the heading “Blosum62 Substitution (score≧0)” in which the substitutions are based on known conservative amino acid substitutions. In another embodiment, one or more of X₁₋₁₄ are substituted as shown in Table B under the heading “Blosum62 Substitution (score>0)” in which the substitutions are based on known conservative amino acid substitutions. In a further embodiment, one or more of X₁₋₁₄ are “conservatively” substituted, meaning that one or more of X₁₋₁₄ is substituted with an art-recognized conservative substitution, either using a BLOSUM62 scoring matrix or another scoring matrix or classification scheme, such as other BLOSUM (Blocks Substitution Matrix) matrices, such as, without limitation BLOSUM 45, 52, 60, 80 or 90 (the higher the number, the less divergent the “matches”), PAM (Point Accepted Mutation) matrices, such as, without limitation, PAM 100, 120, 160, 200 and 250 (the higher the number, the more divergent the “matches”), GONNET matrices and DNA identity matrices. In yet another embodiment, one or more of X₁₋₁₄, or X_{(2-4, 7, 10, 11 or 13)}, are substituted with any amino acid. Lastly, any 1, 2 or 3 or more amino acids listed specifically (not as “Xₙ”) in Formula I may be substituted conservatively or non-conservatively with another amino acid.

In further embodiments, the α-ENaC-derived epithelial sodium channel-inhibitory agent comprises the core amino acid sequence: PHPLQRL (SEQ ID NO: 2, residues 7-13), LPHPLQRL (SEQ ID NO: 2, residues 6-13); PHPLQRLX₆X₇PX₈P (SEQ ID NO: 2, wherein residues 7-18), DX₁X₂X₃X₄X₅PHPLQRLX₆X₇PX₈P (SEQ ID NO: 2, residues 1-18), PHPLQRLX₆X₇PX₈PX₉X₁₀X₁₁X₁₂ (SEQ ID NO: 2, residues 7-22)and PHPLQRLX₆X₇PX₈PX₉X₁₀X₁₁X₁₂RX₁₃X₁₄R (SEQ ID NO: 2, residues 7-26), wherein X₁₋₁₄ are, independently, any amino acid or are substituted in any manner described above. One or more of the C-terminal 1, 2, 3, 4, 5, 6, 7 or 8 amino acids of Formula I may be deleted, substituted, optionally conservatively, with any amino acid, or contain insertions of any amino acid, such as Q. For example, and without limitation, the C-terminal 1, 2, 3, 4, 5, 6 or 7 amino acids may be deleted without affecting the ENaC inhibitory activity of the α-ENaC-derived epithelial sodium channel-inhibitory agent. The C-terminal 1-8 amino acids of Formula I may prove to be unnecessary to inhibit epithelial sodium channel activity because they include the furin protease consensus cleavage site “RXXR (SEQ ID NO: 2, residues 23-26)” and/or show less inter-species conservation. Likewise, one or more of the N-terminal 1, 2, 3, 4, 5, 6 or 7 or more amino acids of Formula I may be deleted, substituted, optionally conservatively, with any amino acid, or contain insertions of any amino acid, because less sequence conservation is seen in the N-terminal 6 amino acids of Formula I. For example, and without limitation, the N-terminal 1, 2, 3, 4, 5, 6 or 7 amino acids may be deleted without affecting the ENaC inhibitory activity of the α-ENaC-derived epithelial sodium channel-inhibitory agent.

Table C provides sequences of the epithelial sodium channel-inhibitory agent cleaved from the γ subunit of the epithelial sodium channel of the listed species during activation of the epithelial sodium channel. Table D identifies commonalities between the γ-ENaC sequences listed in Table C and provides conservative substitutions for those non-conserved amino acids using one common substitution matrix (BLOSUM62).

TABLE C
Gamma ENaC subunit Cleavage Products

| | |
|---|---|
| Mouse | +TL,32 |
| EAGSMRSTWEGTPPRFLNLIPLLVFNENEKGKARDFFTG-RKRK186 | |
| (SEQ ID NO: 6, residues 144-186) | |
| Human | |
| EAESWNSVSEGKQPRFSHRIPLLIFDQDEKGKARDFFTG-WKRK181 | |
| (SEQ ID NO: 5, residues 139-181) | |
| Rat | |
| EAGSMPSTLEGTPPRFFKLIPLLVFNENEKGKARDFFTG-RKRK181 | |
| (SEQ ID NO: 11) | |
| Dog | |
| EAESWSSAWEGTRPKFLRLVPLMVFSQDETSQARDFLTG-RKRK183 | |
| (SEQ ID NO: 12) | |
| Bovine | |
| EAQSWSSVRKGTDPKFLNLAPLMAFEKGDTGKARDFFTG-RKRK183 | |
| (SEQ ID NO: 13) | |
| Rabbit | |
| DTESWSPAWEGVRPKFLNLVPLLIFNRDEKGKARDFLSLGRKRK184 | |
| (SEQ ID NO: 14) | |
| Consensus | |
| EAESWSSXWEGTXPKFLNLIPLLVFNXDEKGKARDFFTGXRKRK | |
| (SEQ ID NO: 1, wherein residues 1 = E, 2 = A, | |
| 3 = E, 5 = W, 6 = S, 7 = S, 9 = W, 10 = E, 12 = T, | |
| 15 = K, 17 = L, 18 = N, 19 = L, 20 = I, 23 = L, | |
| 24 = V, 26 = N, 28 = D, 29 = E, 30 = K, 31 = G, | |
| 32 = K, 37 = F, 38 = T, 39 = G, and 41 = R) | |

TABLE D
Gamma ENaC subunit Consensus:
X₁X₂X₃SX₄X₅X₆X₇X₈X₉GX₁₀X₁₁PX₁₂FX₁₃X₁₄
X₁₅X₁₆PLX₁₇X₁₈FX₁₉X₂₀X₂₁X₂₂X₂₃X₂₄X₂₅ARDFX₂₆X₂₇X₂₈X₃₀X₂₉KRK
(Formula II) (SEQ ID NO: 1)

| | Residue | Endogenously | Blosum62 | Blosum62 |
|---|---|---|---|---|
| | in Mouse | Present | Substitution | Substitution |
| Residue | or Human | Residues | (score ≧ 0)^{a} | (score > 0)^{a} |
| X₁ | E | E, D | E, D, S, T, N, Q, H, R, K | E, D, T, N, Q, K |
| X₂ | A | A, T | A, T, C, S, G, P, N, D, E, Q, H, K | A, T, S, P, G, D |
| X₃ | G, E | G, E, Q | G, E, Q, S, T, A, V, N, D, H, R, K, M | G, E, Q, T, D, K |
| X₄ | M, W | M, W | M, W, Q, I, L, F, Y | M, W, I, L, F, Y |
| X₅ | R, N | R, N, P, S | R, N, P, S, E, Q, H, K, T, G, D, K, A | R, N, P, S, Q, K, D, H, T, A |
| X₆ | S | S, P | S, P, T, A, G, N, D, E, Q, K | S, P, T, A, N |
| X₇ | T, V | T, V, A | T, V, A, S, P, G, N, D, E, Q, H, K, M, I, L, C | T, V, A, S, P, G, D, M, I, L |
| | | | W, S, L, R, F, Y, T, A, G, N, D, E, Q, | W, S, L, R, F, Y, T, A, N, M, I, V, Q, K |
| X₈ | W, S | W, S, L, R | K, M, I, V, H | |
| X₉ | E | E, K | E, K, S, T, N, D, Q, H, R | E, K, D, Q, R |
| X₁₀ | T, K | T, K, V | T, K, V, S, P, G, N, D, E, Q, H, R, A, M, I, L | T, K, V, S, P, G, D, E, Q, R, M, I, L |
| X₁₁ | P, Q | P, Q, R, D | P, Q, R, D, T, S, N, E, K, M, H | P, Q, R, D, T, E, K, N |
| X₁₂ | R | R, K | R, K, N, E, Q, H, S, T | R, K, Q, E |
| X₁₃ | L, S | L, S, F | L, S, F, M, I, V, A, G, N, D, E, Q, K, L, Y | L, S, F, M, I, V, T, A, N, Y |
| X₁₄ | N, H | N, H, K, R | N, H, K, R, T, G, D, E, Q, N, S | N, H, K, R, S, D, Y, E, Q |
| X₁₅ | L, R | L, R | L, R, M, I, V, F, N, E, Q, H, K | L, R, M, I, V, Q, K |
| X₁₆ | I | I, V, A | I, V, A, M, L, F, C, S, G | I, V, A, M, L, S |
| X₁₇ | L | L, M | L, M, I, V, F, Q | L, M, I, V |
| X₁₈ | V, I | V, I, A | V, I, A, M, L, F, C, S, G | V, I, A, M, L, S |
| X₁₉ | N, D | N, D, S, E | N, D, S, E, T, G, Q, R, K, A, H | N, D, S, E, H, T, A, Q, K |
| X₂₀ | E, Q | E, Q, K, R | E, Q, K, R, S, T, N, D, H, M | E, Q, K, R, D |
| X₂₁ | N, D | N, D, G | N, D, G, S, T, E, Q, R, K, A, V | N, D, G, S, H, T, E |
| X₂₂ | E | E, D | E, D, S, T, N, Q, H, R, K | E, D, T, Q, K, N |
| X₂₃ | K | K, T | K, T, S, N, E, Q, R, P, G, D, H | K, T, E, Q, R, S, P, G, D |
| X₂₄ | G | G, S | G, S, T, A, V, A, N, D, E, Q, K | G, S, T, A, N |
| X₂₅ | K | K, Q | K, Q, S, T, N, E, R, D, H, M | K, Q, E, R, K |
| X₂₆ | F | F, L | F, L, M, I, Y, W, V, F | F, L, Y, M, I, V |
| X₂₇ | T | T, S | T, S, P, G, N, D, E, Q, H, K, | T, S, P, G, D, A, N |
| X₂₈ | G | G, L | G, L, S, T, A, V, M, I, F | G, L, T, M, I, V |
| X₂₉ | R, W | R, W | R, W, N, E, Q, H, K, F | R, W, Q, K, F, Y |
| X₃₀ | — | — or G | —, G, T, A, V | —, G, T |

^{a}Includes “Endogenously Present Residues.”

In one embodiment corresponding to the mouse-human examples shown below (mouse polypeptide affecting human epithelial sodium channel), the epithelial sodium channel-inhibitory agent is a polypeptide cleaved from a γ-ENaC subunit or a derivative or analog thereof (γ-ENaC-derived epithelial sodium channel-inhibitory agent), comprising the amino acid sequence: EAGSMRSTWEGTPPRFLNLIPLLVFNENEKGKARDFFTGRKRK (SEQ ID NO: 6, residues 144-186), EAESWNSVSEGKQPRFSHRIPLLIFDQDEKGKARDFFTGWKRK (SEQ ID NO: 5, residues 139-181), or EAX₃SX₄X₅SX₇X₈EGX₁₀X₁₁PRFX₁₃X₁₄X₁₅IPLLX₁₈FX₁₉X₂₀X₂₁EKGKARDFFTGX₂₉KRK (SEQ ID NO: 20), where X₃ is G or E, X₄ is M or W, X₅ is R or N, X₇ is T or V, X₈ is W or S, X₁₀ is T or K, X₁₁ is P or Q, X₁₃ is L or S, X₁₄ is N or H, X₁₅ is L or R, X₁₈ is V or I, X₁₉ is N or D, X₂₀ is E or Q, X₂₁, is N or D, X₂₉ is R or W. In yet another embodiment, the polypeptide has the mammalian consensus sequence:
X₁X₂X₃SX₄X₅X₆X₇X₈X₉GX₁₀X₁₁PX₁₂FX₁₃X₁₄X₁₅X₁₆PLX₁₇X₁₈FX₁₉X₂₀X₂₁X₂₂X₂₃X₂₄X₂₅ARDFX₂₆X₂₇X₂₈X₃₀X₂₉KRK(Formula II)(SEQ ID NO: 1), or
EAESWSSX₇WEGTX₁₁PKFLNLIPLLVFNX₂₀DEKGKARDFFTGX₃₀RKRK (SEQ ID NO: 1, wherein residues 1=E, 2=A, 3=E, 5=W, 6=S, 7=S, 9=W, 10=E, 12=T, 15=K, 17=L, 18=N, 19=L, 20=I, 23=L, 24=V, 26=N, 28=D, 29=E, 30=K, 31=G, 32=K, 37=F, 38=T, 39=G and 41=R) in which one or more of X₁₋₃₀ are independently substituted as shown in Table D under the heading “Endogenously Present Residues.” In a further embodiment, one or more of X₁₋₂₉ are substituted as shown in Table D under the heading “Blosum62 Substitution (score≧0)” in which the substitutions are based on known conservative amino acid substitutions. In another embodiment, one or more of X₁₋₃₀ are substituted as shown in Table D under the heading “Blosum62 Substitution (score>0)” or in which the substitutions are based on known conservative amino acid substitutions. In a further embodiment, one or more of X₁₋₃₀ are “conservatively substituted,” as defined above. In yet another embodiment, one or more of X₁₋₃₀, X_{(2, 3, 4, 7, 8, 10-15, 18-25 or 27-30)}, or X_{(3, 7, 8, 10, 13 and 30)} are substituted with any amino acid, or in the case of X₃₀, no amino acid. Lastly, any 1, 2 or 3 or more amino acids listed specifically (not as “Xₙ”) in Formula II may be substituted conservatively or non-conservatively with another amino acid.

In further embodiments, the γ-ENaC-derived epithelial sodium channel-inhibitory agent comprises the amino acid sequence of Formula II, but in which 1, 2, 3, 4, 5, 6, 7 or more C-terminal amino acids of Formula II may be deleted, substituted, optionally conservatively, with any amino acid, or contain insertions of any amino acid, such as Q. For example, and without limitation, the C-terminal 1, 2, 3, 4, 5, 6 or 7 amino acids may be deleted without affecting the ENaC inhibitory activity of the γ-ENaC-derived epithelial sodium channel-inhibitory agent. The C-terminal 1-7 or more amino acids of Formula II may prove to be unnecessary to inhibit epithelial sodium channel activity because they include the CAP protease consensus cleavage signal. Likewise, one or more of the N-terminal 1, 2, 3, 4, 5, 6 or 7 or more amino acids, of Formula II may be deleted, substituted, optionally conservatively, with any amino acid, or contain insertions of any amino acid. For example, and without limitation, the N-terminal 1, 2, 3, 4, 5, 6 or 7 amino acids may be deleted without affecting the ENaC inhibitory activity of the γ-ENaC-derived epithelial sodium channel-inhibitory agent.

The described epithelial sodium channel-inhibitory agents can be 26 or 43 amino acid residues in length (in the case of the α- or γ-epithelial sodium channel-derived agents, respectively). However, longer and shorter agents are contemplated as being useful in the methods described herein. In the case of shorter agents, those deletions are described above. In the case of longer agents, additional amino acids, amino acid analogs or polypeptide sequence(s) may be added to the above-described 26-mers (α-ENac) and 43-mers (γ-ENaC) or described shorter versions thereof. In the case of the addition of additional polypeptide sequence(s), the additional polypeptide sequence(s) comprise, in one non-limiting embodiment, any polypeptide containing a consensus or core sequence described above and/or may comprise additional amino acids or amino acid analogs, such as contiguous amino acids of the epithelial sodium channel subunit to which the agent corresponds. The agent also may include, without limitation, useful antigenic tags (such as poly-His tags) or secretion signal peptides, as are known in the art, that would be useful in production and/or affinity purification of a recombinantly-prepared polypeptide. The antigenic tag or signal sequence may be cleavable or not cleavable from the agent, in a manner well-known in the art. The length of the agent or polypeptide corresponding to the α ENaC subunit may be from 5 to 100 amino acids in length, 10-50 amino acids in length, or 20-35 amino acids in length, though any effective length may be useful. The length of the agent or polypeptide corresponding to the γ ENaC subunit may be from 7 to 100 amino acids in length, 10-75 amino acids in length, or 35-50 amino acids in length, though any effective length may be useful. Multiple iterations of the α or γ sequences described above may be provided in a single compound, either attached covalently, in frame by a traditional peptide linkage, cross-linked either randomly or specifically by any useful cross-linking technology (for example and without limitation, those available from Pierce Biotechnology, Inc. of Rockford, Ill. or Toronto Research Chemicals, North York, Ontario, Canada) or non-covalently.

As used herein, an epithelial sodium channel-inhibitory agent may be said to comprise an amino acid sequence, meaning, if the agent is a polypeptide, it comprises a sequence of traditional amino acids and if it is a peptide analog, it comprises the equivalent of the stated amino acid sequence. For example and without limitation, a polypeptide or peptoid comprising the sequence PHPLQRL (SEQ ID NO: 2, residues 7-13), refers to a polypeptide comprising the sequence PHPLQRL (SEQ ID NO: 2, residues 7-13) as well as to peptoids, for example and without limitation, NProNHisNProNLeuNGlnNArgNLeu and NProHisProLeuGlnArgLeu (SEQ ID NO: 2, residues 8-13, in which residue 8 is modified with an NPro). Likewise, an “agent consisting of 43 amino acids” refers to a 43-mer polypeptide as well as a 43-mer polypeptoid. In contrast, a “polypeptide consisting of 43 amino acids” refers only to a polypeptide chain of common L-amino acids (Ala, Cys, Asp, Glu, Phe, Gly, His, Ile, Leu, Met, Asn, Pro, Gln, Arg, Ser, Thr, Val, Trp or Tyr), unless indicated otherwise. Further, unless stated otherwise, amino acids referenced herein in the context of a particular sequence of an “agent” can be traditional amino acids as well as amino acid analogs.

Depending on the disease or condition to be treated, an epithelial sodium channel-inhibitory agent described herein may be administered to a patient in a composition (drug product) comprising the epithelial sodium channel-inhibitory agent in any quantity effective to treat the patient by inhibiting the patient's epithelial sodium channel. In the case of its use as a diuretic, the polypeptide or analog thereof may be administered orally or by parenteral delivery routes, such as, without limitation, intravenous (iv), intraperitoneal (ip), intramuscular (im), intratumoral (it) and subcutaneous (sc) routes. In the case of delivery of the polypeptide to the airway to treat COPD, for example and without limitation, an effective amount of the epithelial sodium channel-inhibitory agent typically will be delivered as an aerosol by any suitable aerosol or spray delivery system, for example and without limitation, one of the many commercially-available aerosol or spray inhalant drug delivery systems. Nasal delivery also can be achieved by nasal spray, for instance using a spray device commercially available from Ing. Erich Pfeiffer GmbH of Radolfzell, Germany. The agent may be delivered as a liquid or as a dry powder and may be formulated in any suitable composition, whether dry or liquid, according to methods known to those of skill in the formulary arts.

“Effective amounts” of the drug will vary, depending on the dosage route, the disease or condition to be treated, the severity of the patient's condition or disease, the drug product's bioavailability and the persistence of the active agent in the patient's airway. In any case, an “effective amount” of any given drug is an amount of that drug necessary to create a desired effect in a patient with the secondary goal of minimizing toxicity to the patient, thereby achieving acceptable toxicity with maximum therapeutic effect. As is known in the pharmaceutical arts, often therapeutic levels may be achieved by multiple smaller doses, as opposed to one large bolus. In one embodiment, from about 100 pg to about 100 mg of the active agent, including any increments therebetween, may be delivered to a patient in a single dose. Doses may be administered, for example and without limitation, from about one to about ten times daily, typically from one to about four times daily, one to seven times a week, or as needed. The drug product may be administered for a fixed time period, for example and without limitation, one, two, three, or four days or weeks, any number of months, or a year or more, including increments therebetween. In its use as an expectorant to treat infectious diseases of the airways, such as viral or bacterial rhinitis, pharyngitis, laryngitis, bronchitis, bronchiolitis and/or pneumonia, doses of one to four times daily for about three days to about four weeks may be preferred in order to enhance clearance from the airway.

The composition (drug product) may be prepared in any manner acceptable in the pharmaceutical and/or formulary arts, and may include any suitable, pharmaceutically-acceptable excipients, including, without limitation: water, buffers, salts, acids, bases, permeation enhancers, oils, vitamins, herbal or other natural extracts, fatty acids, phospholipids, colorings, chelating agents, sweeteners and flavorings.

In another embodiment, methods of characterizing the activity of an epithelial sodium channel-inhibitory agent are provided. The methods comprise testing an epithelial sodium channel-inhibitory agent described herein for inhibition of epithelial sodium channel activity and quantifying the degree of inhibition, optionally against other compound(s), such as, without limitation: amiloride, other epithelial sodium channel inhibiting agent(s) or other α- or γ-ENaC-derived epithelial sodium channel-inhibitory agent(s) described herein. A number of methods of testing for epithelial sodium channel activity are known and are readily available, but one simple method is the method described below; namely determining the effect of a given polypeptide or derivative described above on amiloride-sensitive current in epithelial sodium channel-expressing Xenopus oocytes. Other methods, such as measuring amiloride-sensitive short circuit currents in epithelial monolayers mounted in an Ussing chamber, measuring equivalent short circuit currents in epithelial monolayers using an EVOM device (World Precision Instruments), or measuring nasal, airway or rectal potential differences would be equally suitable for testing of the epithelial sodium channel-inhibitory agents.

### EXAMPLE 1

### Alpha Peptide

Oocyte Expression—cRNAs for α, β, γ mouse ENaC (wild-type and mutant) subunits and mouse prostasin were synthesized with T3 or T7 m-Message mMachineTM (Ambion, Austin, Tex.). Subunits carrying furin cleavage consensus site mutations and the corresponding wild-type subunits have amino-terminal HA and carboxyl-terminal V5 tags (Hughey, R. P., Bruns, J. B., Kinlough, C. L., Harkleroad, K. L., Tong, Q., Carattino, M. D., Johnson, J. P., Stockand, J. D., and Kleyman, T. R. (2004) J. Biol. Chem. 279, 18111-18114 18). Stage V-VI Xenopus laevis oocytes were pretreated with 1.5 mg/ml of type IV collagenase and injected with 0.5-2 ng of cRNA/subunit. Injected oocytes were maintained as previously reported (Carattino, M. D., Hill, W. G., and Kleyman, T. R. (2003), Arachidonic Acid Regulates Surface Expression of Epithelial Sodium Channels, J Biol. Chem. 278, 36202-36213 29).

Peptides—Peptides were synthesized and purified by the Peptide Synthesis Facility of the Molecular Medicine Institute, University of Pittsburgh or by GenScript Corporation. The sequences of the peptides were: α-26, DLRGALPHPLQRLRTPPPPNPARSAR (SEQ ID NO: 4, residues 206-231); scrambled α-26 (SCR), PLTQPRPRLADHNPLSARRRPLPAGP (SEQ ID NO: 15); P/A (α-26 with all Pro substituted with Ala), DLRGALAHALQRLRTAAAANAARSAR (SEQ ID NO: 16); R/E (α-26 with all Arg substituted with Glu), DLEGALPHPLQELETPPPPNPAESAE (SEQ ID NO: 17). Peptides were amino-terminal acetylated and carboxylterminal amidated.

Two-electrode Voltage Clamp—Two-electrode voltage clamp (TEV) was performed as previously described (Carattino, M. D., Hill, W. G., and Kleyman, T. R. (2003) J Biol. Chem. 278, 36202-36213 29). The extracellular solution (TEV solution) was (in mM): 110 NaCl, 2 KCl, 1.54 CaCl₂, 10 HEPES, pH 7.4, or otherwise as indicated. The ENaC-mediated component of the whole cell Na⁺ current was determined by bath perfusion with TEV solution supplemented with 10 μM amiloride. ENaC-mediated whole cell Na⁺ currents, at −60 mV,were defined as the amiloride-sensitive component of the current. Experiments with oocytes co-expressing ENaC and prostasin and the corresponding controls were performed in the presence of aprotinin (1 μM) to impede any potential degradation of α-26 by prostasin.

mpkCCDc14 Cell Culture—mpkCCDc14 cells were grown as previously reported (Bens, M., Vallet, V., Cluzeaud, F., Pascual-Letallec, L., Kahn, A., Rafestin-Oblin, M. E., Rossier, B. C., and Vandewalle, A. (1999), Corticosteroid-dependent sodium transport in a novel immortalized mouse collecting duct principal cell line, J. Am. Soc. Nephrol. 10, 923-934). mpkCCDc14 cells were subcultured onto permeable filter supports coated with collagen (0.4-μm pore size, 1-cm² surface area, Snapwell filters; Coming Inc., Acton, Mass.). Cells were kept on filters for at least 4 days in defined medium (Bens, M., Vallet, V., Cluzeaud, F., Pascual-Letallec, L., Kahn, A., Rafestin-Oblin, M. E., Rossier, B. C., and Vandewalle, A. (1999) J. Am. Soc. Nephrol. 10, 923-934 30) that was changed every second day. At least 24 h prior to the experiments, the culture medium was replaced with a minimal medium (without drugs or hormones) that contained only Dulbecco's modified Eagle's medium and Ham's F-12.

Primary Cultures of HAE Cells—HAE cells were obtained from excess pathological tissue remaining after lung transplantation or from organ donor lungs deemed not suitable for transplantation under a protocol approved by the University of Pittsburgh Investigational Review Board. All cells were isolated from second through sixth generation bronchi and grown on permeable Transwell supports (Coming Inc.) as previously described (Devor, D. C., and Pilewski, J. M. (1999), UTP inhibits Na⁺ absorption in wild-type and DeltaF508 CFTR-expressing human bronchial epithelia, Am. J. Physiol. 276, C827-C837 31. ).

Short Circuit Current Measurements—Cell culture inserts were mounted in modified Ussing chambers (Harvard Apparatus, Holliston, Mass.), and the monolayers were continuously short-circuited with a voltage/current clamp system (Physiologic Instruments, San Diego, Calif.). A 2-mV bipolar pulse was applied periodically to measure the resistance of the monolayer. As previously described by Bridges et al. (Bridges, R. J., Newton, B. B., Pilewski, J. M., Devor, D. C., Poll, C. T., and Hall, R. L. (2001), Na+ transport in normal and CF human bronchial epithelial cells is inhibited by BAY 39-9437, Am. J. Physiol. 281, L16-L23 32, Na⁺ transport in normal and CF human bronchial epithelial cells is inhibited by BAY 39-9437), the bath solution contained (in mM) 120 NaCl, 25 NaHCO₃, 3.3 KH₂PO₄, 0.8 K₂HPO₄, 1.2 MgCl₂, 1.2 CaCl₂, and 10 glucose. The pH of this solution was 7.4 when gassed with a mixture of 95% O2-5% CO₂ at 37° C.

Single Channel Studies—Bath and pipette solutions contained (in mM) 110 LiCl, 2 CaCl₂, 2 KCl, and 10 Hepes, pH 7.4. Experiments were performed as previously described (Carattino, M. D., Sheng, S., and Kleyman, T. R. (2005), Mutations in the pore region modify epithelial sodium channel gating by shear stress, J Biol. Chem. 280, 4393-4401 33). Single channel currents were recorded in the cell-attached configuration of patch clamp, acquired at 5 kHz and filtered at 1 kHz by a 4-pole low pass Bessel filter. Single channel currents were further filtered at 100-200 Hz with a Gaussian filter for display and analysis. Recordings were performed at an applied pipette potential of +60 mV.

Western Blot—ENaC was immunoprecipitated from extracts of transiently transfected MDCK cells with goat anti-V5 antibody conjugated to agarose from Novus Biologicals (Littleton, Colo.) and immunoblotted for the α subunit with mouse anti-V5 monoclonal antibody from Invitrogen as previously described (Hughey, R. P., Mueller, G. M., Bruns, J. B., Kinlough, C. L., Poland, P. A., Harkleroad, K. L., Carattino, M. D., and Kleyman, T. R. (2003), Maturation of the epithelial Na⁺ channel involves proteolytic processing of the alpha- and gamma-subunits, J. Biol. Chem. 278, 37073-37082 17).

Data and Statistical Analyses—and mean time of inhibition are expressed as the mean with a 95% confidence interval (CI); otherwise, data were expressed as the mean ±S.E. (n), where “n” equals the number of independent experiments analyzed. IC₅₀ was estimated from normalized currents plotted as a function of the peptide concentration fitted, as shown in Equation 1
y=t/(1+10^{(logX-log/C50)}) (Eq. 1)
where y is the response variable, X is the concentration of peptide, and IC₅₀ is the concentration of peptide that provokes a response half way between the baseline (t) and maximum response. The time course of inhibition in oocytes was estimated with the Equation 2, which described an exponential decay from a plateau
y=m+(p−m)e^{(-k(T-T0)} (Eq. 2)
where y is the response variable, p and m are the plateau and maximal responses, k is the rate constant, T₀ is the beginning of exponential decay, and T is time. For statistical analyses the normality and equality of the standard deviations of the data were tested. Based on these results a parametric or a nonparametric test was used. Fitting and statistical comparisons were performed with Sigmaplot 8.02 (SPSS Inc., Chicago, Ill.) and GraphPad 3.0 (GraphPad Software, San Diego, Calif.).
Results

The Lack of Furin Cleavage Destabilizes the Open State of ENaC—We previously reported that the α subunit of ENaC is cleaved twice, immediately following two furin consensus cleavage sites, at Arg-205 and Arg-231 (Hughey, R. P., Bruns, J. B., Kinlough, C. L., Harkleroad, K. L., Tong, Q., Carattino, M. D., Johnson, J. P., Stockand, J. D., and Kleyman, T. R. (2004) J. Biol. Chem. 279,18111-18114 18). A third potential furin consensus cleavage site at Arg-208 within the α subunit is not processed by furin (Hughey, R. P., Bruns, J. B., Kinlough, C. L., Harkleroad, K. L., Tong, Q., Carattino, M. D., Johnson, J. P., Stockand, J. D., and Kleyman, T. R. (2004) J. Biol. Chem. 279, 18111-18114 18). The introduction of Ala mutations at all three Arg in the α subunit (αR205A,R208A,R231A (αRtripleA)) prevented α subunit cleavage and significantly reduced the expression of ENaC whole cell currents in Xenopus oocytes (Hughey, R. P., Bruns, J. B., Kinlough, C. L., Harkleroad, K. L., Tong, Q., Carattino, M. D., Johnson, J. P., Stockand, J. D., and Kleyman, T. R. (2004) J. Biol. Chem. 279, 18111-18114 18). Whole cell currents measured in oocytes expressing wild-type or furin-insensitive mutant channels (αRtripleAβγ) increased to similar levels in response to external trypsin, suggesting that ENaC subunit proteolysis increases channel open probability rather than channel number (Hughey, R. P., Bruns, J. B., Kinlough, C. L., Harkleroad, K. L., Tong, Q., Carattino, M. D., Johnson, J. P., Stockand, J. D., and Kleyman, T. R. (2004) J. Biol. Chem. 279, 18111-18114 18). At the single channel level we confirmed that mutant αRtripleA had a markedly reduced open probability in the presence of a high external [Na⁺] (Sheng, S., Carattino, M. D., Bruns, J. B., Hughey, R. P., and Kleyman, T. R. (2006) Am. J. Physiol. 290, F1488-F1496 28). Furthermore, Caldwell et al. (Caldwell, R. A., Boucher, R. C., and Stutts, M. J. (2004) Am. J. Physiol. 286, C190-C194 Caldwell, R. A., Boucher, R. C., and Stutts, M. J. (2005) Am. J. Physiol. 288, L813-L819 25, 26) have shown that α subpopulation of ENaCs with a low open probability and brief mean open times are converted by external trypsin or elastase to channels that have characteristic long open and closed times.

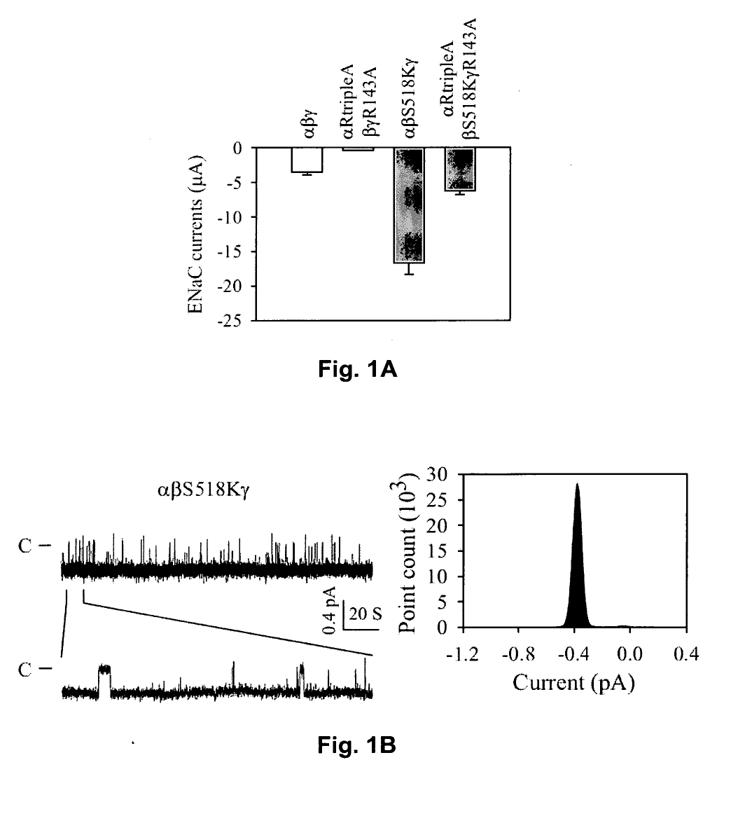

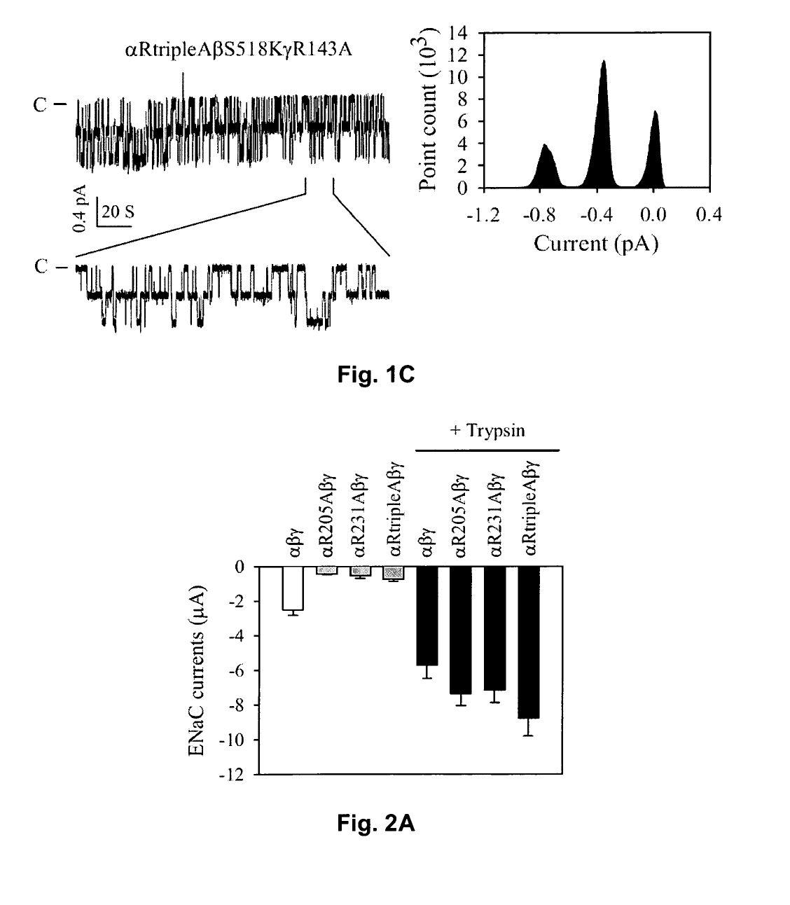

Previous studies have shown that ENaC gating is modulated, in part, via a regulatory domain that has been localized to the region preceding the second membrane-spanning (pre-M2) domain (Sheng, S., Li, J., McNulty, K. A., Kieber-Emmons, T., and Kleyman, T. R. (2001), Second transmembrane domains of ENaC subunits contribute to ion permeation and selectivity, J. Biol. Chem. 276, 1326-1334 and Snyder, P. M., Bucher, D. B., and Olson, D. R. (2000), Gating induces a conformational change in the outer vestibule of ENaC, J. Gen. Physiol. 116, 781-790). Channels with a specific mutation at a key site (the degenerin site) within the pre-M2 region of the β subunit (βS518K) have a very high open probability (Carattino, M. D., Edinger, R. S., Grieser, H. J., Wise, R., Neumann, D., Schlattner, U., Johnson, J. P., Kleyman, T. R., and Hallows, K. R. (2005), Epithelial sodium channel inhibition by AMP-activated protein kinase in oocytes and polarized renal epithelial cells, J. Biol. Chem. 280, 17608-17616 35, 36), whereas channels that lack proteolysis of the α subunit have a very low open probability (Sheng, S., Carattino, M. D., Bruns, J. B., Hughey, R. P., and Kleyman, T. R. (2006) Am. J. Physiol. 290, F1488-F1496 28). We used these mutants to examine whether the lack of proteolysis, or the introduction of a mutation at the degenerin site, exerted a dominant effect on ENaC activity when expressed in oocytes. Oocytes were co-injected with either (i) wild-type αβγ, (ii) wild-type β with a (αRtripleA) and γ (γR143A) subunits carrying mutations in the furin cleavage sites, (iii) wild-type α and γ with a β subunit degenerin mutation (βS518K), or (iv) three mutant subunits (αRtripleAβS518KγR143A). We found that oocytes expressing αRtripleAβS518KγR143A exhibited whole cell Na⁺ currents that were similar in magnitude to currents measured in oocytes expressing wild-type αβγ (p>0.05). These currents were significantly greater than the currents measured in oocytes expressing αRtripleAβγR143A (p<0.001) and significantly less than currents measured in oocytes expressing αβS518Kγ (p<0.05, n=24-26) (Kruskal-Wallis test (non-parametric ANOVA) followed by Dunn's multiple comparisons test) (FIG. 1A). At the single channel level, αRtripleAβS518KγR143A channels had a single channel conductance of 6.2±0.1 pS (n=5-6) similar to what we described previously for wild-type αβγ (Carattino, M. D., Sheng, S., and Kleyman, T. R. (2005), Mutations in the pore region modify epithelial sodium channel gating by shear stress, J. Biol. Chem. 280, 4393-4401 33). However, analysis of αRtripleAβS518KγR143A at a single channel level showed frequent short openings and closures (FIG. 1C), whereas the αβS518Kγ mutant had a high open probability with long openings and brief closures (FIG. 1B). These data provide additional evidence in support of a major role for proteolytic processing of ENaC extracellular domains in the regulation of channel gating.

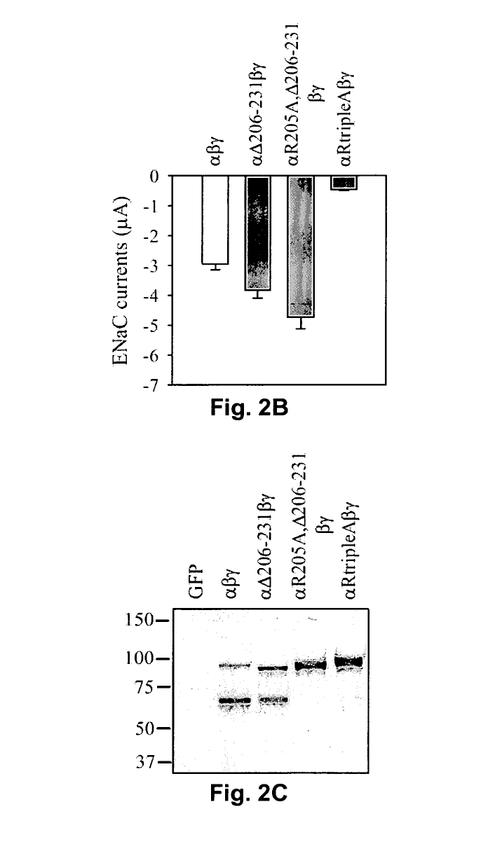

Proteolytic Activation of ENaC Requires Cleavage at Multiple Sites within the α Subunit—As there are two furin cleavage sites within the α subunit, we examined whether the presence of a single furin cleavage site was sufficient for activation of ENaC. Xenopus oocytes expressing ENaCs with mutations at the proximal (αR205Aβγ), distal (αR231Aβγ), or at both furin cleavages sites (αR205A/R208A/R231Aβγ (αRtripleA)) exhibited significantly reduced amiloride-sensitive currents when compared with oocytes expressing wild-type channels (FIG. 2A). Subsequent treatment of these oocytes expressing either wild-type or mutant ENaCs with trypsin (2 μg/ml) increased amiloridesensitive currents to similar levels (FIG. 2A), suggesting that similar numbers of channels were present on the plasma membrane in each case. These data support our previous finding that the α subunit must be cleaved twice during ENaC maturation for channels to exhibit normal activity (Sheng, S., Carattino, M. D., Bruns, J. B., Hughey, R. P., and Kleyman, T. R. (2006) Am. J. Physiol. 290, F1488-F1496 28).

The mechanism by which proteolysis increases ENaC open probability has not been elucidated. It is possible that non-cleaved subunits have an overall constrained structure that increases residency time in the closed state and/or reduces residency time in the open state. Alternatively, as the α subunit must be cleaved twice during ENaC maturation for channels to exhibit normal activity, the tract between both furin cleavages sites might be functioning as an inhibitor that acts by stabilizing the closed conformation of the channel. To discriminate between these two possibilities, we deleted the tract of amino acids that are putatively excised from the α subunit following furin processing (Asp-206-Arg-23 1). This mutant (αΔ206-231) still contains the first furin consensus site after Arg-205 and should be cleaved once. In addition, we generated an α subunit mutant with both the R205A mutation and deletion of the tract Asp-206-Arg-231 (R205A,Δ206-231) that should lack furin-dependent processing. ENaC currents were not significantly affected by the Δ206-231 mutation in the α subunit (p >0.05) and were even modestly increased in the absence of α subunit furin cleavage (αR205A,Δ206-231 mutant) versus wild-type αβ (p<0.005, Kruskal-Wallis test (nonparametric ANOVA) followed by Dunn's multiple comparisons test). When co-expressed in MDCK cells with β and γ subunits, αΔ206-231 migrated slightly faster on SDS gels than wildtype α subunit as expected with the deletion of 26 amino acid residues (FIG. 2C), whereas both wild-type αβγ and αΔ206-231βγ exhibited evidence of α subunit cleavage (i.e. appearance of a carboxyl-terminal polypeptide of 65 kDa) consistent with previously described furin-dependent processing (Hughey, R. P., Mueller, G. M., Bruns, J. B., Kinlough, C. L., Poland, P. A., Harkleroad, K. L., Carattino, M. D., and Kleyman, T. R. (2003) J. Biol. Chem. 278, 37073-37082 and Hughey, R. P., Bruns, J. B., Kinlough, C. L., Harkleroad, K. L., Tong, Q., Carattino, M. D., Johnson, J. P., Stockand, J. D., and Kleyman, T. R. (2004) J. Biol. Chem. 279,18111-18114 17, 18). In contrast, no evidence of α subunit cleavage was observed when αR205A,Δ206-231βγ was expressed in MDCK cells (FIG. 2C). Thus, in channels lacking the α subunit tract Asp-206-Arg-231, proteolytic processing by furin is not required to activate the channel, indicating that this tract may function as an inhibitor that stabilizes the channel in the closed conformation. Ourdata suggest that a double cleavage event is required to dissociate a putative blocker (tract αAsp-206-Arg-231) from the channel. In the absence of this tract, the α subunit does not need to be cleaved for the channel to display normal activity.

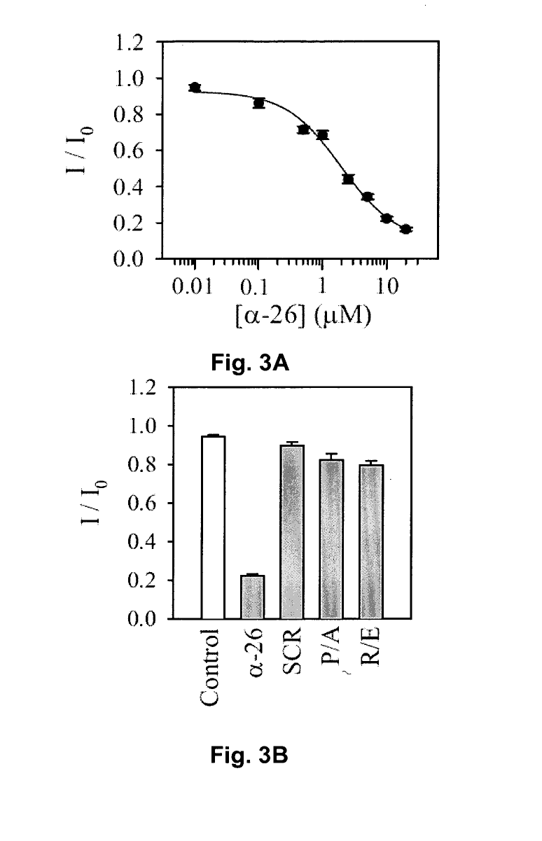

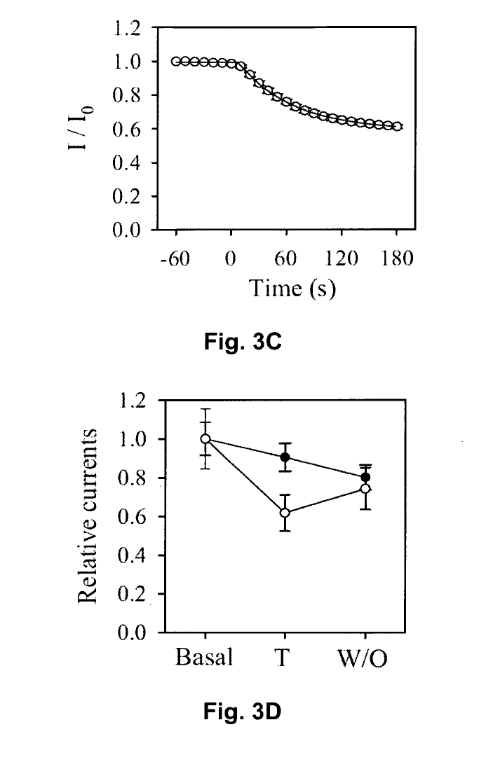

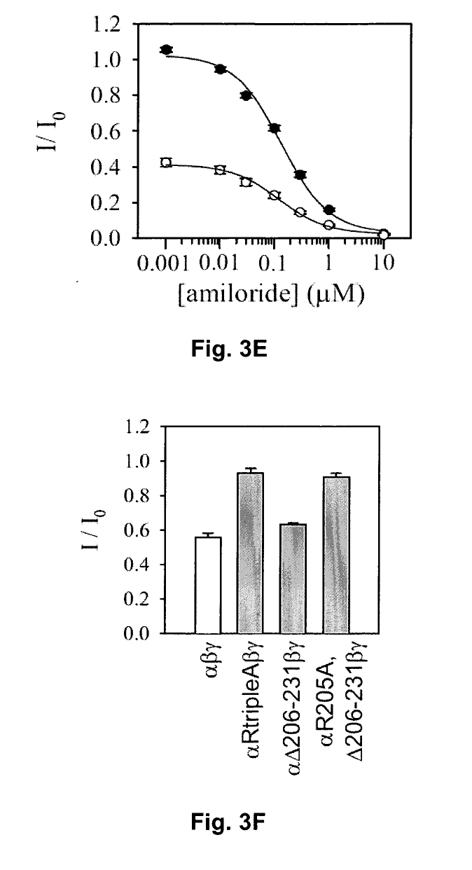

ENaC Is Inhibited by a Short Peptide Derived from Its Proteolytic Processing—As consequence of proteolytic processing of the α subunit, a peptide of 26 residues, corresponding to Asp-206-Arg-231, is predicted to be cleaved from the α subunit. We synthesized this 26-mer peptide (α-26) and examined whether it altered channel activity. Wildtype αβγ expressed in oocytes were inhibited by α-26 with an IC₅₀ of 2.8×10⁻⁶ M (CI, 2.5 to 3.2×10⁻⁶ M) (FIG. 3A), but not by a control peptide with the sequence scrambled (FIG. 3B, SCR). IC₅₀s determined at holding potentials of −20, −60, and −100 mV were 2.9×10⁻⁶ M (CI, 2.5 to 3.3×10⁻⁶ M), 2.8×10⁻⁶ M (CI, 2.5 to 3.2×10⁻⁶ M) and 2.9×10⁻⁶ M (CI, 2.6 to 3.4×10⁻⁶ M), respectively, indicating that the block of ENaC was not voltage dependent. The time required for 1 μM α-26 to achieve half maximal inhibition was 42 s (CI, 36.8-48.8 s) (FIG. 3C), suggesting that α-26 has a slow association rate when compared with amiloride, a prototypic voltage-dependent channel blocker that achieves half maximal inhibition within seconds (Kleyman, T. R., and Cragoe, E. J., Jr. (1988), Amiloride and its analogs as tools in the study of ion transport, J Membr Biol 105(1), 1-21).α-26 was a reversible inhibitor of the channel (FIG. 3D). The IC₅₀ for amiloride block of ENaC was not affected by the presence of α-26. The amiloride IC₅₀ was 152 nM (CI, 116-198 nM) in the presence of 2.5 μMα-26 and 150 nM (CI, 136-165 nM) in the absence of peptide (FIG. 3E), suggesting that α-26 does not bind to or interact with the amiloride binding site. α-26 is rich in Pro and Arg residues. Substitution of all Pro with Ala, or alternatively all Arg with Glu, led to a significant reduction in the block of ENaC by 10 μM peptide (FIG. 3B). Channels that lack furin-dependent cleavage of the α subunit (αRtripleAβγ), as well as channels lacking both furin-dependent cleavage of the α subunit and the tract αAsp-206-Arg-231 (i.e. αR205A,Δ206-231βγ) were not blocked by 1 μM α-26 (FIG. 3F), indicating that the peptide does not have access to its effector binding site within the channel in the absence of α subunit cleavage.

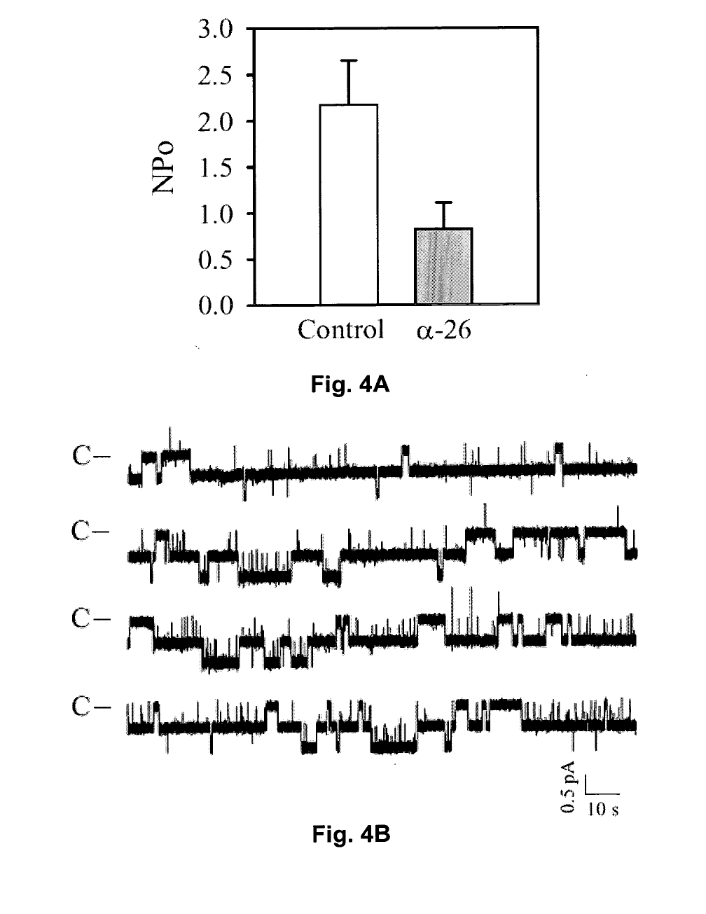

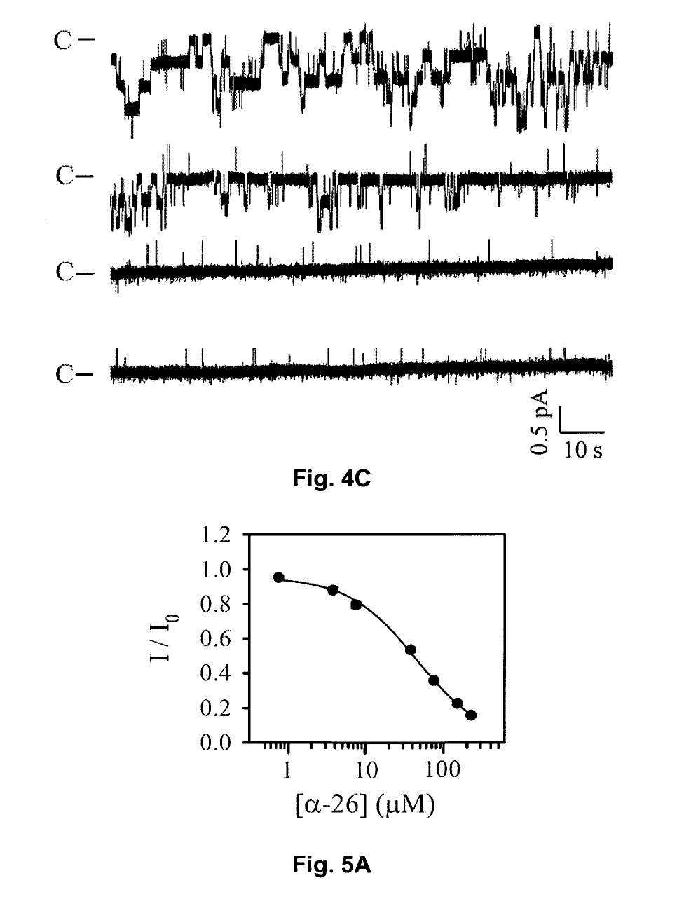

Single channel analyses using a patch pipette backfilled with α-26 (10 μM) demonstrated a reduction in the product of the number of channels and open probability (NPo) compared with controls (FIG. 4A). For experiments performed with α-26, the tip of the pipette was filled with standard patch solution. Pipette resistances were similar in control (7.8±0.4 MΩ) and α-26-treated patches (6.9±0.6 MΩ) (p=0.30, unpaired t test). Control patches that did not contain α-26 in the pipette remained active over the recording period (FIG. 4B). Unitary currents, at an applied pipette potential of +60 mV, were similar in both control (0.42±0.2 pA, n=5) and α-26-containing patches (0.39±0.2 pA, n=7) (p=0.32, unpaired t test). FIG. 4C shows a representative tracing recorded with α-26 in the pipette. At the beginning of the experiment open times were similar to that observed in controls (FIG. 4B). Over the course of the experiment openings decreased and were shorter in duration compared with control until channels were fully closed. Assuming no changes in the number of channels during the recording, the changes observed in NPo due to the presence of α-26 in the pipette largely reflect a change in channel P₀.

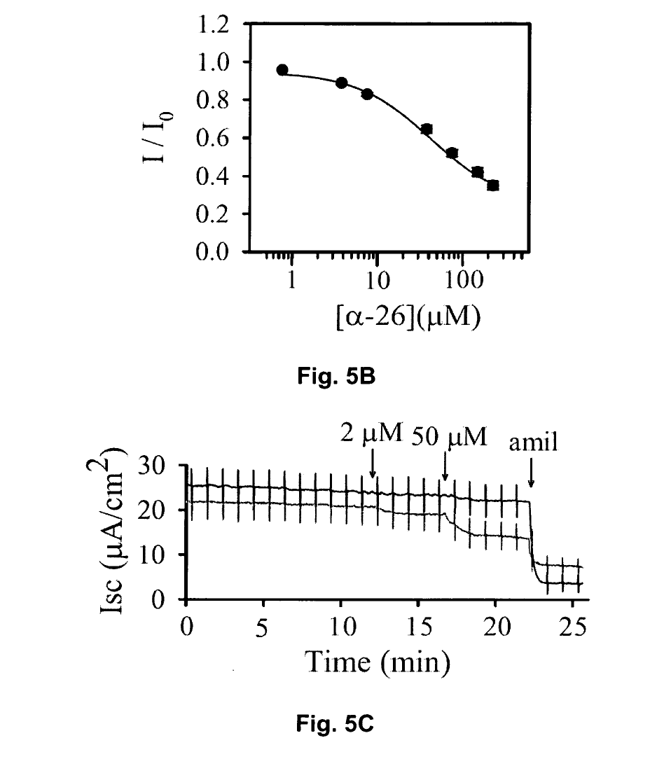

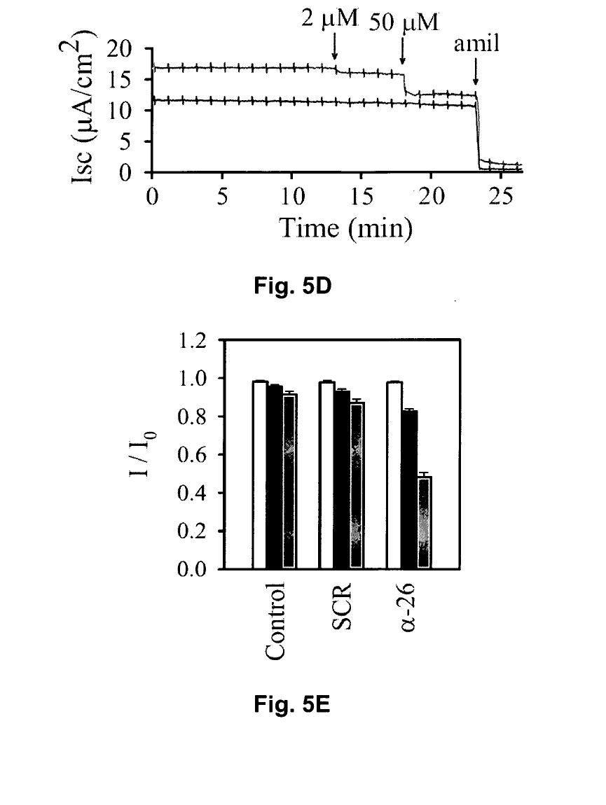

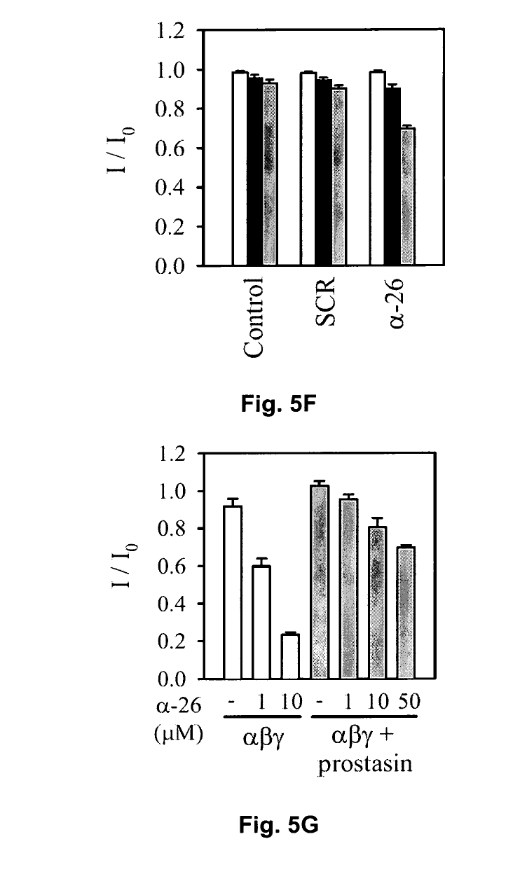

Endogenous ENaCs Are Inhibited by α-26—We examined whether α-26 inhibited endogenous channels expressed in a primary culture of HAE cells and in a cell line derived from mouse CCDs (mpkCCD_{c14}) by monitoring the effects of α-26 on the amiloride-sensitive component of the short circuit current (Isc).α-26 inhibited Isc in HAE (FIG. 5A, 5C and 5E) and mpkCCD_{c14} (FIG. 5B, 5D and 5F) monolayers with IC₅₀s of 4.6×10⁻⁵M (CI, 4.2 to 4.9×10⁻⁵M) and 1.0×10⁻⁴M (CI, 9.2 to 1.1×10⁻⁴M), respectively (FIG. 5A and 5B). The scrambled α-26 peptide (50 μM) did not inhibit Isc measured in HAE or in mpkCCD_{c14} monolayers (FIGS. 5C and 5F).

Prostasin Reduces the Efficacy of α-26—The differences in the IC₅₀s of α-26 for ENaCs expressed in oocytes versus HAE and mpkCCD_{c14} monolayers may represent differential proteolytic processing of ENaC subunits. ENaC is cleaved and activated by furin in Xenopus oocytes and MDCK and Chinese hamster ovary cells (Hughey, R. P., Bruns, J. B., Kinlough, C. L., Harkleroad, K. L., Tong, Q., Carattino, M. D., Johnson, J. P., Stockand, J. D., and Kleyman, T. R. (2004) J. Biol. Chem. 279, 18111-18114 18), whereas other serine proteases, in addition to furin, are likely involved in the proteolytic processing and activation of ENaC in other mammalian epithelial cells (Bridges, R. J., Newton, B. B., Pilewski, J. M., Devor, D. C., Poll, C. T., and Hall, R. L. (2001) Am. J. Physiol. 281, L16-L23 32, 38 Vuagniaux, G., Vallet, V., Jaeger, N. F., Pfister, C., Bens, M., Farman, N., Courtois- Coutry, N., Vandewalle, A., Rossier, B. C., and Hummler, E. (2000), Activation of the amiloride-sensitive epithelial sodium channel by the serine protease mCAP1 expressed in a mouse cortical collecting duct cell line, J. Am. Soc. Nephrol. 11, 828-834 ). The mechanisms for ENaC activation by proteases other than furin are unknown. Kunitz-type protease inhibitors that do not block furin activity reduced amiloride-sensitive currents in some cultured epithelial cells (Vallet, V., Chraibi, A., Gaeggeler, H. P., Horisberger, J. D., and Rossier, B. C. (1997), An epithelial serine protease activates the amiloride-sensitive sodium channel, Nature 389, 607-610 20, Bridges, R. J., Newton, B. B., Pilewski, J. M., Devor, D. C., Poll, C. T., and Hall, R. L. (2001), Na+ transport in normal and CF human bronchial epithelial cells is inhibited by BAY 39-9437, Am. J. Physiol. 281, L16-L23 32, 38; Vuagniaux, G., Vallet, V., Jaeger, N. F., Pfister, C., Bens, M., Farman, N., Courtois- Coutry, N., Vandewalle, A., Rossier, B. C., and Hummler, E. (2000), Activation of the amiloride-sensitive epithelial sodium channel by the serine protease mCAP1 expressed in a mouse cortical collecting duct cell line, J. Am. Soc. Nephrol. 11, 828-834 ). Prostasin is a serine protease that is blocked by Kunitz-type protease inhibitors and activates ENaC when co-expressed in oocytes (Vallet, V., Chraibi, A., Gaeggeler, H. P., Horisberger, J. D., and Rossier, B. C. (1997) Nature 389, 607-610 20). cRNAs for wild-type ENaC subunits (0.5 ng/subunit) were co-injected with or without prostasin cRNA (1 ng). Co-expression of prostasin and ENaC in oocytes led to an increase in amiloride-sensitive currents (−9.6±1.3 μA, n=28) compared with oocytes expressing wildtype ENaC alone (−5.3±0.6 μA, n=26) (p<0.005, unpaired t test with Welch correction). Interestingly, co-expression of prostasin and ENaC significantly reduced the efficacy of α-26-dependent inhibition of ENaC (FIG. 5G).

Additional studies have been performed to define regions within the α-26 peptide that are required for its inhibitors effect. We observed that the 8-residue fragment LPHPLQRL (SEQ ID NO: 2, residues 6-13) inhibited mouse ENaCs expressed in oocytes with an IC₅₀ of ˜1 micromolar.

### EXAMPLE2

### Gamma Peptide

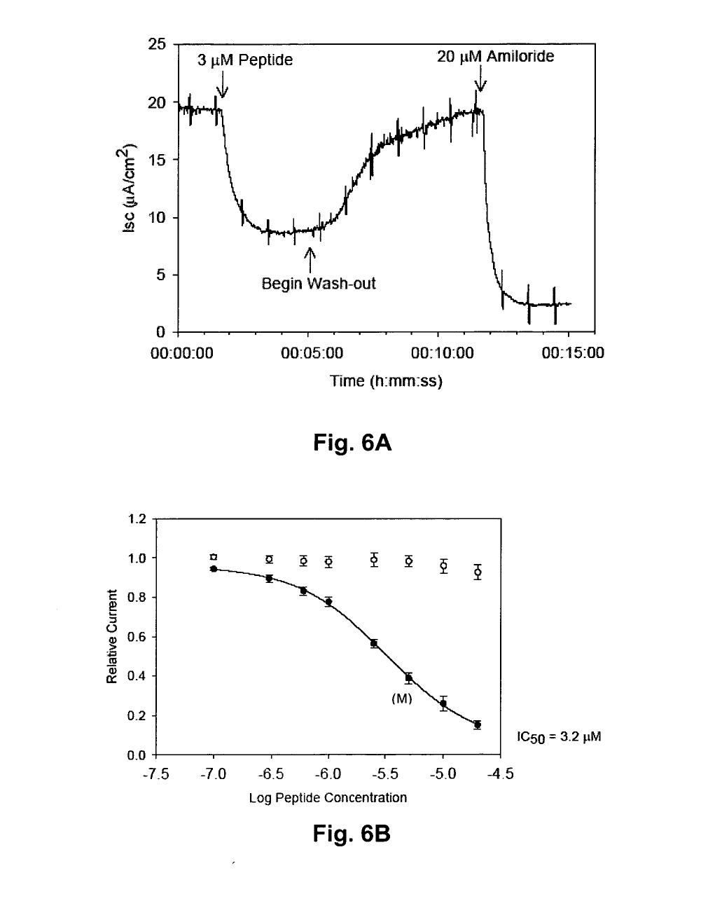

The γ-peptide reversibly inhibits endogenous ENaC in a mouse cortical collecting duct (mpkCCD_{c14}) cell line. (FIG. 6A) mpkCCD_{c14}cells cultured on permeable membrane supports and mounted in a modified Ussing chamber were continuously short-circuited by a voltage clamp amplifier (Physiologic Instruments, San Diego, Calif.). Transepithelial resistance was monitored by applying a 4 mV bipolar pulse every 60 seconds. Where indicated, a 43 residue peptide corresponding to E144-K186 of mouse γ-ENaC (γ-peptide) was added to the apical chamber at a final concentration of 3 μM. This led to a marked inhibition of transepithelial ion transport which was relieved by wash out of the peptide. Application of 20 μM amiloride to the apical chamber then confirmed that this was ENaC mediated transepithelial sodium transport. (FIG. 6B) mpkCCD_{c14}cells cultured and voltage clamped as above were exposed to increasing concentrations of either the γ-peptide (closed circles) or a scrambled peptide (FKGFVGKEALREILTFWLRFNNTEMDSKPLRTRANPPSKGGRE (SEQ ID NO: 18), open circles). While the scrambled peptide failed to inhibit amiloride sensitive short circuit currents, the γ-peptide inhibited in a dose dependant manner. Nonlinear curve fitting provided an estimated IC₅₀ of 3.2+/−0.2 μM. Data presented as mean +/−standard error (n=8).

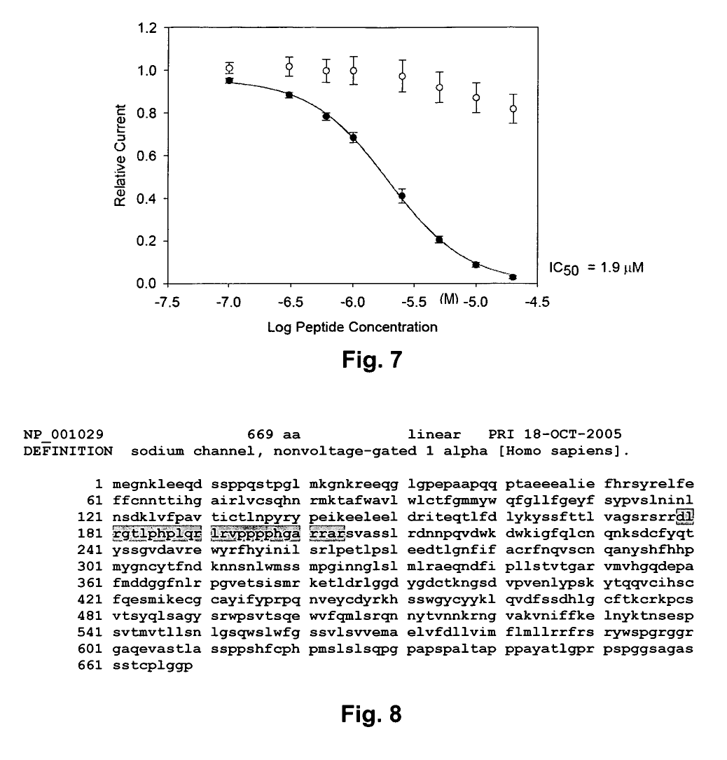

The γ-peptide is a potent inhibitor of ENaC mediated transepithelial sodium transport in human airway epithelial (HAE) cells. Primary cultures of HAE cells grown on permeable membrane supports and mounted in modified Ussing chambers were continuously short-circuited by a voltage clamp amplifier (Physiologic Instruments, San Diego, Calif.). Monolayers were exposed to increasing concentrations of either the γ-peptide (closed circles) or a scrambled peptide (open circles). As shown in FIG. 7, while the scrambled peptide showed only a slight inhibition of amiloride sensitive currents consistent with a time-dependant rundown of channel activity, the γ-peptide caused strong inhibition in a dose dependant manner. Nonlinear curve fitting provided an estimated IC₅₀ of 1.9 μM. (n=8-9).

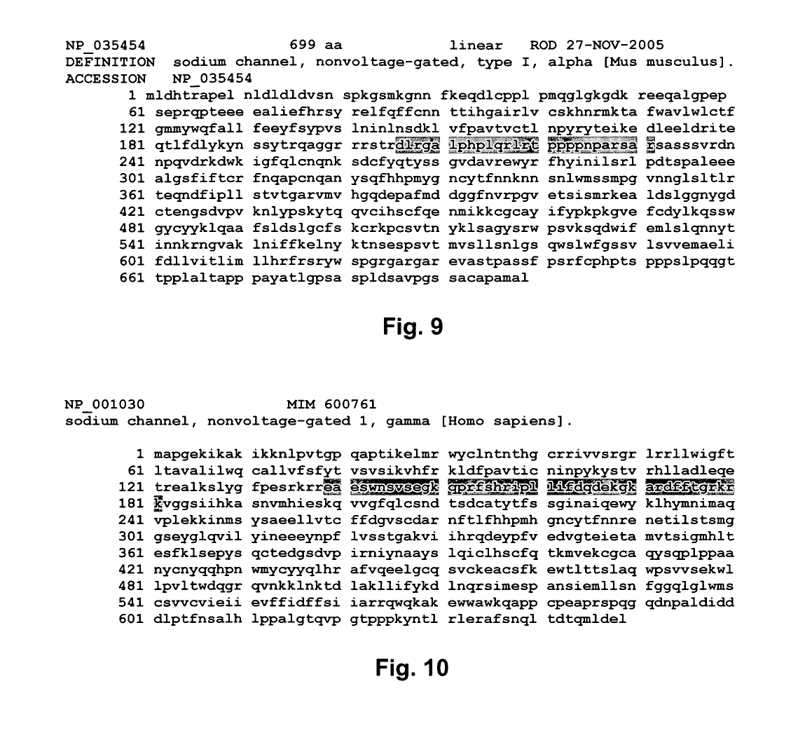

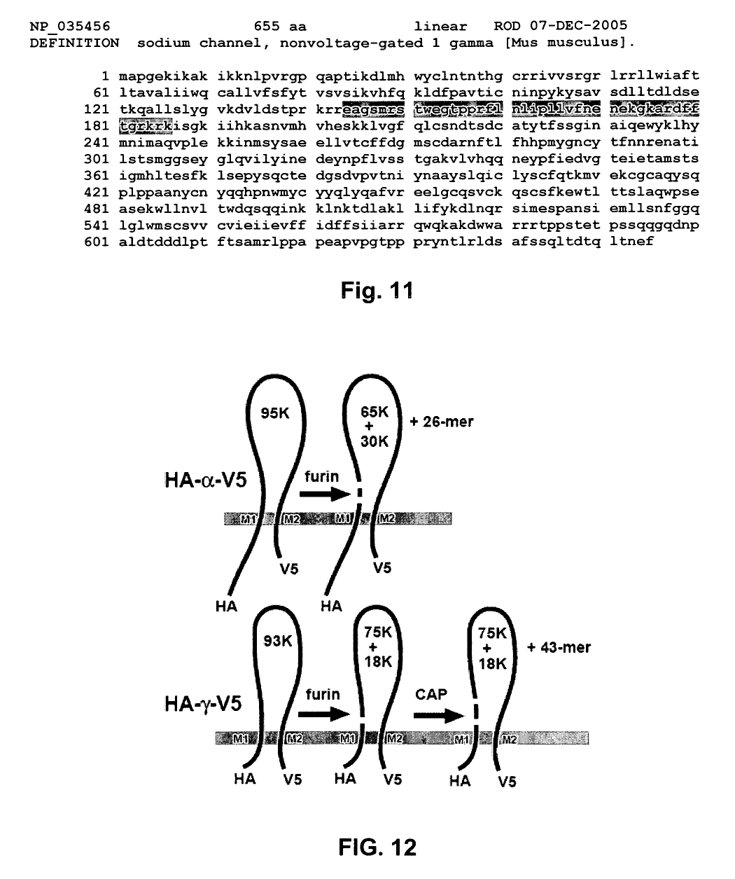

Sequences. Epithelial sodium channel α-subunits may be found in the public Genbank database under Accession Nos.: human, NP_{—}001029 (FIG. 8, OMIM No. 600228, SEQ ID NO: 3) and P37088; mouse, NP_{—}035454 (FIG. 9, SEQ ID NO: 4), AAD21244 and AF112185; rat, CAA49905 (cleaved sequence shown in SEQ ID NO: 8); bovine, P55270 (cleaved sequence shown in SEQ ID NO: 9); and rabbit, Q9N1333 (cleaved sequence shown in SEQ ID NO: 10). Epithelial sodium channel γ-subunits may be found in the public Genbank database under Accession Nos.: human, NP_{—}001030 (FIG. 10, OMIM No. 600761, SEQ ID NO: 5) and P51170; mouse, NP_{—}035456 (FIG. 11, SEQ ID NO: 6), AAD21246 and AF112187; rat, CAA54905 (cleaved sequence shown in SEQ ID NO: 11); dog, XP_{—}875216 (cleaved sequence shown in SEQ ID NO: 12); bovine (partial sequence), XP_{—}875216 (cleaved sequence shown in SEQ ID NO:13); and rabbit, Q28738 (cleaved sequence shown in SEQ ID NO: 14). Cleaved amino acid sequences are highlighted in FIGS. 8, 9, 10 and 11.

### EXAMPLE 3

### Gamma Peptide, Additional Studies.

Experimental Procedures

Vectors and Cell Culture—Wild-type, C-terminal and double epitope tagged mouse ENaC subunit cDNAs were previously described (Hughey, R. P., Mueller, G. M., Bruns, J. B., Kinlough, C. L., Poland, P. A., Harkleroad, K. L., Carattino, M. D., and Kleyman, T. R. (2003) J Biol Chem 278, 37073-37082 and Ahn, Y. J., Brooker, D. R., Kosari, F., Harte, B. J., Li, J., Mackler, S. A., and Kleyman, T. R. (1999), Cloning and functional expression of the mouse epithelial sodium channel, Am J Physiol 277, F121-129). The γ subunit mutations were generated in pcDNA3.1 (+) using a standard two-step PCR method. A cDNA clone (IMAGE #: 3600399) encoding full length mouse prostasin was obtained from Open Biosystems (Huntsville, Ala.) and sub-cloned into pcDNA3.1 (+) (Invitrogen, Carlsbad, Calif.). MDCK type 1 cells were a gift from Barry M. Gumbiner (Memorial Sloan-Kettering Cancer Center, New York) and were cultured as previously described (Lavelle, J. P., Negrete, H. O., Poland, P. A., Kinlough, C. L., Meyers, S. D., Hughey, R. P., and Zeidel, M. L. (1997), Low permeabilities of MDCK cell monolayers: a model barrier epithelium, Am J Physiol 273, F67-75). CCD (mpkCCD_{c14}) cells were a gift from Alain Vandewalle (Institut National de la Santéet de la Recherche Médicale, Paris, France) and were cultured as previously described (Bens, M., Vallet, V., Cluzeaud, F., Pascual-Letallec, L., Kahn, A., Rafestin-Oblin, M. E., Rossier, B. C., and Vandewalle, A. (1999) J Am Soc Nephrol 10, 923-934). HAE cells were isolated and cultured as described (Devor, D. C., Bridges, R. J., and Pilewski, J. M. (2000), Pharmacological modulation of ion transport across wild-type and DeltaF508 CFTR-expressing human bronchial epithelia, Am J Physiol Cell Physiol 279, C461-479).

Transient Transfection and Immunoblot Analysis of MDCK Cells—MDCK cells were transiently transfected with mENaC cDNAs using LipofectAMINE 2000 as described by the manufacturer (Invitrogen). The α and β subunits were C-terminally epitope tagged with Myc and FLAG, respectively. The γ subunit (wild-type or mutant) had N-terminal HA and C-terminal V5 epitope tags. Where indicated, mouse prostasin cDNA was co-transfected. Total amount of DNA transfected was held constant by co-transfection of GFP. Twenty-four hours post transfection, cells were lysed and the γ subunit was immunoprecipitated and immunoblotted as described previously Hughey, R. P., et al. (2003) J Biol Chem 278, 37073-37082.

Functional Expression in Xenopus Oocytes—ENaC expression in Xenopus oocytes and two-electrode voltage clamp were performed as previously reported (Carattino, M. D., Sheng, S., Bruns, J. B., Pilewski, J. M., Hughey, R. P., and Kleyman, T. R. (2006), The epithelial Na+ channel is inhibited by a peptide derived from proteolytic processing of its alpha subunit, J. Biol. Chem. 281, 18901-18907; Bruns, J. B., Hu, B., Ahn, Y. J., Sheng, S., Hughey, R. P., and Kleyman, T. R. (2003), Multiple epithelial Na+ channel domains participate in subunit assembly, Am J Physiol Renal Physiol 285, F600-609 and Carattino, M. D., Hill, W. G., and Kleyman, T. R. (2003), Arachidonic acid regulates surface expression of epithelial sodium channels, J Biol Chem 278, 36202-3621). Wild type α and β along with double-tagged γ mENaC cRNAs (1 ng per subunit) were injected with or without 3 ng of mouse prostasin cRNA. Electrophysiological measurements were performed 24 h post injection. The difference in measured current in the absence and presence of amiloride (10 to 20 μM) was used to identify ENaC mediated currents.

Single channel studies—Patch clamp experiments were performed as previously described (Carattino, M. D., Sheng, S., and Kleyman, T. R. (2005) J Biol Chem 280, 4393-4401). Single channel currents were acquired at 5 kHz, and filtered at 1 kHz by a 4-pole low pass Bessel filter. For display and analysis, single channel currents were further filtered at 100-200 Hz with a Gaussian filter. Single channel experiments in oocytes were performed in the cell-attached configuration of patch clamp with identical bath and pipette solutions containing (in mM) 110 LiCl, 2 CaCl₂, 1.54 KCl, and 10 Hepes, pH 7.4. The bath electrode consisted of an Ag—AgCl pellet connected to the bathing solution via an agar bridge made up in 200 mM NaCl. Liquid junction potentials were not corrected. Open probabilities were estimated from amplitude histograms obtained from recordings of at least 6 min of duration at an applied pipette potential of 60 mV using pCLAMP 6 (Axon Instruments, Forster City, Calif.).

Peptides—Peptides were synthesized and HPLC-purified by the peptide synthesis facility of the University of Pittsburgh's Molecular Medicine Institute. The sequences of the peptides in single letter amino acid code were: γ cleavage product (γ-43), EAGSMRSTWEGTPPRFLNLIPLLVFNENEKGKARDFFTGRKRK (SEQ ID NO: 6, residues 144-186); scrambled peptide, FKGFVGKEALREILTFWLRFNNTEMDSKPLRTRANPPSKGGRE (SEQ ID NO: 18). Both peptides were modified by N-terminal acetylation and C-terminal amidation.

Short Circuit Current Recordings—Cells cultured on permeable membrane supports and mounted in modified Costar Ussing chambers were continuously short-circuited by a voltage clamp amplifier (Physiologic Instruments, San Diego, Calif.) as previously described by Butterworth et al (Butterworth, M. B., Edinger, R. S., Johnson, J. P., and Frizzell, R. A. (2005), Acute ENaC stimulation by cAMP in a kidney cell line is mediated by exocytic insertion from a recycling channel pool, J Gen Physiol 125, 81-101).

Statistical Analysis—Data are presented as means±S.E. Significance comparisons between groups were performed with unpaired Student's t tests unless otherwise indicated. A p value of less than 0.05 was considered statistically different. IC₅₀ data are presented as the mean with a 95% confidence interval (CI) and were estimated from normalized currents plotted as a function of the peptide concentration fitted with the following equation y=b+(t−b)/(1+10^{(logIC50-X)n}) where y is the response variable, X is the concentration of peptide, n is the Hill coefficient and IC₅₀ is the concentration of peptide that provokes a response half way between baseline (b) and maximum response (t). Fitting and statistical comparisons were performed with Sigmaplot 8.02 (SPSS Inc, Chicago, Ill.), GraphPad 3.0 (GraphPad Software, San Diego, Calif.) and Clampfit 9.0 (Axon Instruments Inc., Union City, Calif.).

Results

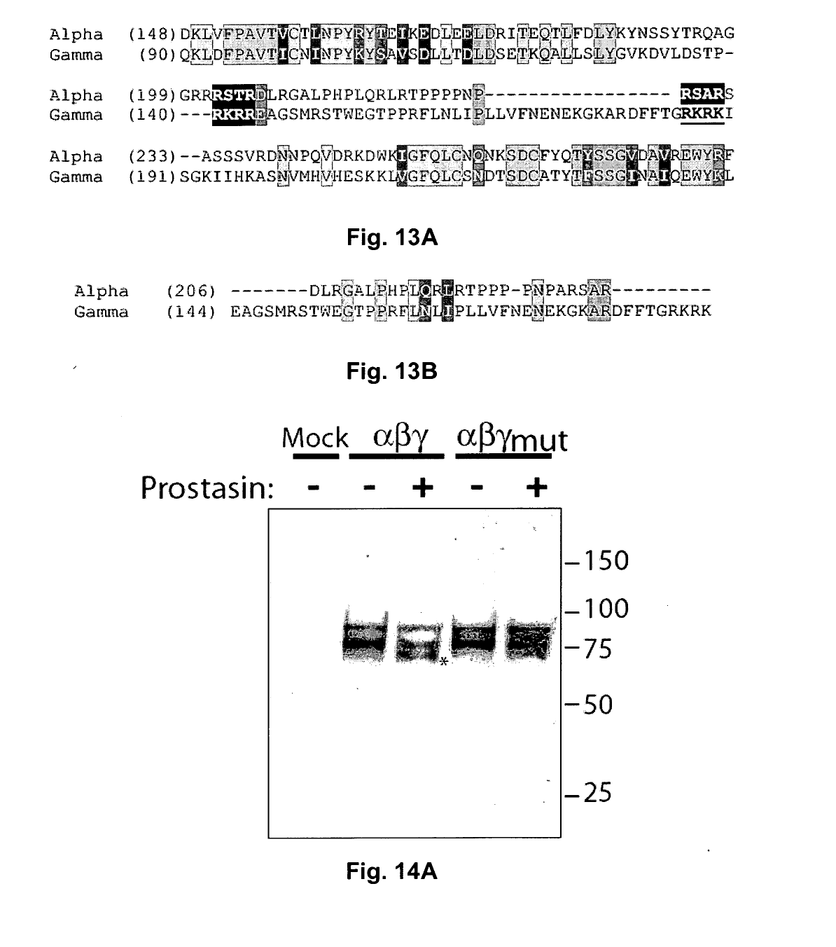

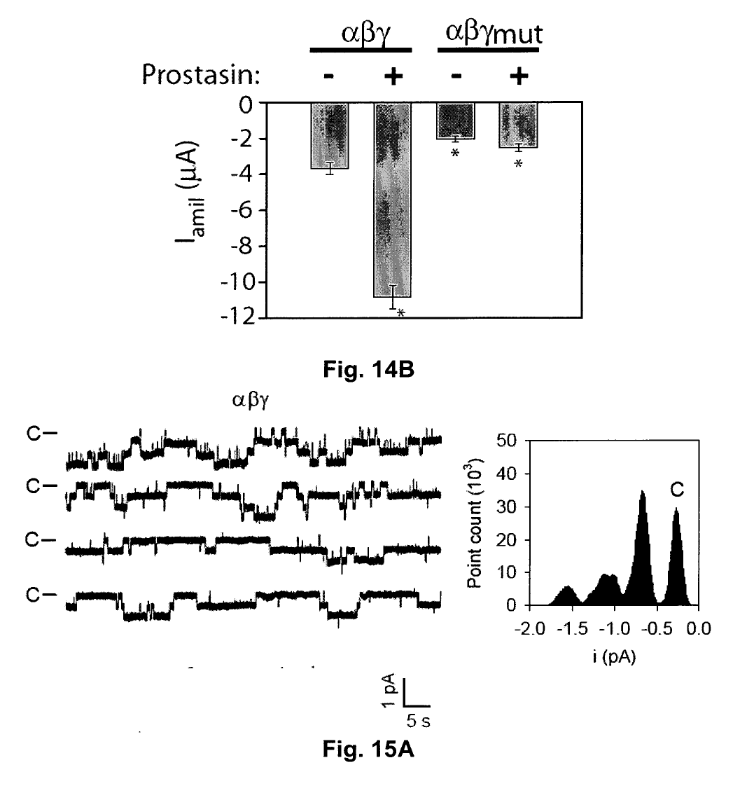

We recently demonstrated that the α subunit of ENaC must be cleaved twice, at two furin cleavage consensus sites, in order to activate the channel (Hughey, R. P., Bruns, J. B., Kinlough, C. L., Harkleroad, K. L., Tong, Q., Carattino, M. D., Johnson, J. P., Stockand, J. D., and Kleyman, T. R. (2004) J Biol Chem 279, 18111-18114; Sheng, S., Carattino, M. D., Bruns, J. B., Hughey, R. P., and Kleyman, T. R. (2006) Am J Physiol Renal Physiol 290, F1488-1496 and Carattino, M. D., et al. (2006) J. Biol. Chem. 281, 18901-18907). This double cleavage event excises a 26-mer inhibitory peptide (Carattino, M. D., et al. (2006) J. Biol. Chem. 281, 18901-18907). The γ subunit is cleaved once by furin at a site that aligns closely to the first α subunit furin site (FIG. 13). Carboxyl-terminal to the single γ subunit furin site is a tetra-basic tract (RKRK¹⁸⁶ (SEQ ID NO: 7)), which aligns closely to the distal furin site in the α subunit, but does not fit the consensus cleavage sequence for furin (Rockwell, N. C., Krysan, D. J., Komiyama, T., and Fuller, R. S. (2002), Precursor processing by kex2/furin proteases, Chem Rev 102, 4525-4548). This sequence may serve as a site for proteolysis by other serine proteases that cleave after basic residues. Prostasin is a serine protease that has been previously shown to activate ENaC when co-expressed in heterologous systems (Vuagniaux, G., Vallet, V., Jaeger, N. F., Hummler, E., and Rossier, B. C. (2002), Synergistic activation of ENaC by three membrane-bound channel-activating serine proteases (mCAP1, mCAP2, and mCAP3) and serum- and glucocorticoid-regulated kinase (Sgk1) in Xenopus Oocytes, J Gen Physiol 120, 191-201; Vallet, V., Chraibi, A., Gaeggeler, H. P., Horisberger, J. D., and Rossier, B. C. (1997) Nature 389, 607-610; Vuagniaux, G., Vallet, V., Jaeger, N. F., Pfister, C., Bens, M., Farman, N., Courtois-Coutry, N., Vandewalle, A., Rossier, B. C., and Hummler, E. (2000), Activation of the amiloride-sensitive epithelial sodium channel by the serine protease mCAP1 expressed in a mouse cortical collecting duct cell line, J Am Soc Nephrol 11, 828-834 and Adachi, M., Kitamura, K., Miyoshi, T., Narikiyo, T., Iwashita, K., Shiraishi, N., Nonoguchi, H., and Tomita, K. (2001), Activation of epithelial sodium channels by prostasin in Xenopus oocytes, J Am Soc Nephrol 12, 1114-1121). Previous studies suggest that a tetra-basic (or dibasic) tract could serve as α substrate for prostasin cleavage (Shipway, A., Danahay, H., Williams, J. A., Tully, D. C., Backes, B. J., and Harris, J. L. (2004), Biochemical characterization of prostasin, a channel activating protease, Biochem Biophys Res Commun 324, 953-963). We therefore examined whether co-expression of ENaC and prostasin altered proteolysis of the γ subunit. ENaCs were expressed in MDCK cells with the γ subunit bearing a carboxyl-terminal V5 epitope tag. Following cell lysis, immunoprecipitation and subsequent immunoblotting with an anti-V5 antibody, we observed both full length (93 kDa) and furin-cleaved (75 kDa) γ subunit (FIG. 14A). When ENaCs were co-expressed with prostasin, an additional γ subunit species of 70 kDa was observed consistent with cleavage at a site carboxyl-terminal to the furin cleavage site. When the RKRK¹⁸⁶ (SEQ ID NO: 7) tract in the γ subunit was mutated to QQQQ¹⁸⁶ ((SEQ ID NO: 19)_{γmut}) only the full length and 75 kDa fragment were observed both in the absence and presence of prostasin expression. These data suggest that prostasin induces cleavage of the γ subunit at the RKRK¹⁸⁶ (SEQ ID NO: 7) tract.

Co-expression of ENaC and prostasin led to a significant, 3-fold increase in whole cell amiloride-sensitive Na⁺ currents in Xenopus oocytes, compared to oocytes expressing ENaC alone (FIG. 14B). Expression of channels with the γ subunit RKRK¹⁸⁶ (SEQ ID NO: 7)/QQQQ (SEQ ID NO: 19) mutation led to a significant 45% reduction in whole cell Na⁺ currents, compared to oocytes expressing wild type ENaCs. Furthermore, co-expression of this mutant ENaC with prostasin led to only a modest, but non-significant increase in Na⁺ currents when compared to oocytes expressing the mutant channels alone (p>0.05). These results suggest that prostasin-induced cleavage at the RKRK186 (SEQ ID NO: 7) site plays a major role in prostasin-dependent activation of ENaC.

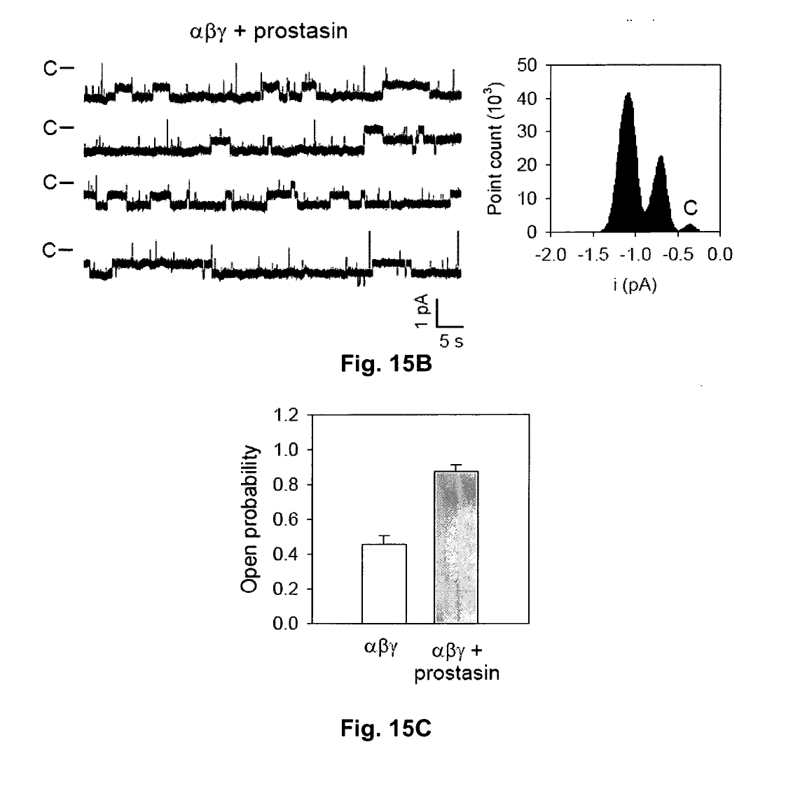

Previous reports, based on a cell surface expression assay in Xenopus oocytes, have inferred that prostasin induces activation of ENaC through an increase in single channel open probability (Vuagniaux, G., et al. (2002) J Gen Physiol 120, 191-201; Vallet, V., et al. (1997) Nature 389, 607-610 and Vuagniaux, G., et al. (2000) J Am Soc Nephrol 11, 828-834). We examined the open probability of wild-type ENaCs expressed in Xenopus oocytes with or without prostasin co-expression. In contrast to the observation of Adachi et al. ((2001), Activation of epithelial sodium channels by prostasin in Xenopus oocytes, J Am Soc Nephrol 12, 1114-1121), channel open probability increased 89% from 0.46±0.05 (n=11) to 0.87±0.04 (n=7, p<0.0001) when ENaC was co-expressed with prostasin (FIG. 15). Prostasin co-expression had no effect on unitary currents at an applied pipette potential of +60 mV (0.41±0.01 pA vs. 0.39±0.01 pA, p=0.33)

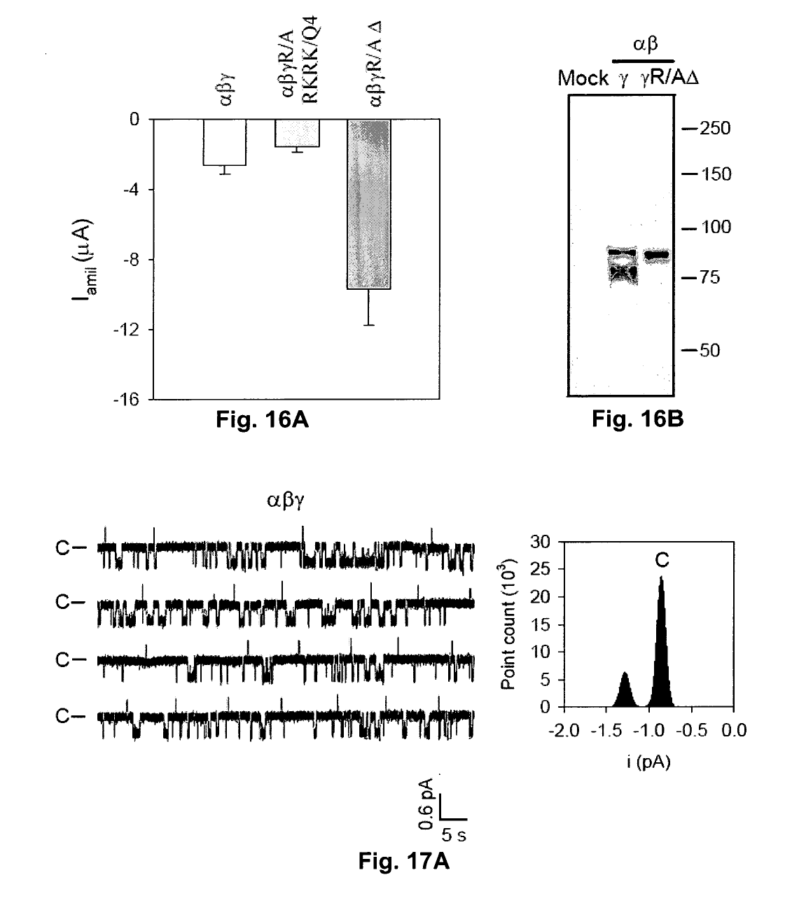

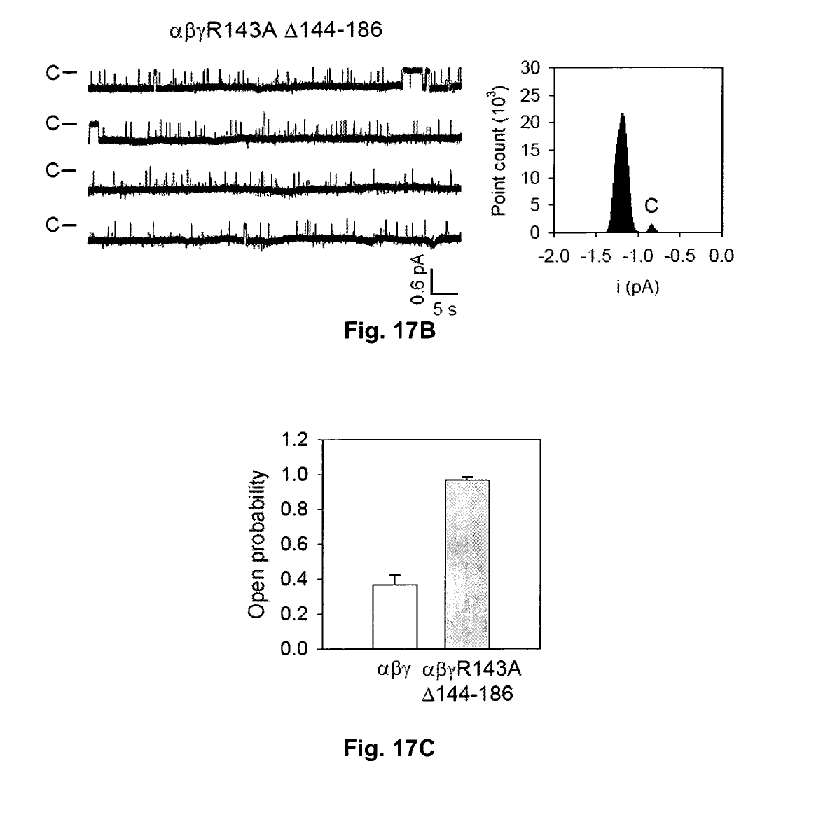

We have recently reported that cleavage of ENaC at two sites within the α subunit activates the channel by releasing an inhibitory 26-mer peptide (Carattino, M. D., et al. (2006) J. Biol. Chem. 281, 18901-18907). We therefore sought to determine if the 43-mer tract in the γ subunit (Glu144-Lys186), predicted to be excised following co-expression with prostasin, also functions as an inhibitory domain. We first examined whether deletion of the 43-mer tract in the γ subunit would enhance channel activity. In addition to removing the tract, Arg143 within the γ furin site was mutated to Ala to prevent furin-dependent cleavage. This mutant γR143A,Δ144-186 (γR/AΔ) was not cleaved when co-expressed with α and β subunits in MDCK cells (FIG. 16B). However, when this mutant was co-expressed with α and β in oocytes, whole cell Na⁺ currents were 3.7-fold greater than currents recorded in oocytes expressing wild type αβγ (FIG. 16A). At a single channel level, the mutant channel was constitutively active, with an open probability of >0.95, compared to wild type channels that had an open probability of 0.37±0.06 (FIG. 17). Unitary currents were not different between mutant (0.39+0.03 pA) and wild type (0.40±0.01) channels at an applied pipette potential of +60 mV (n=4, p=0.75).

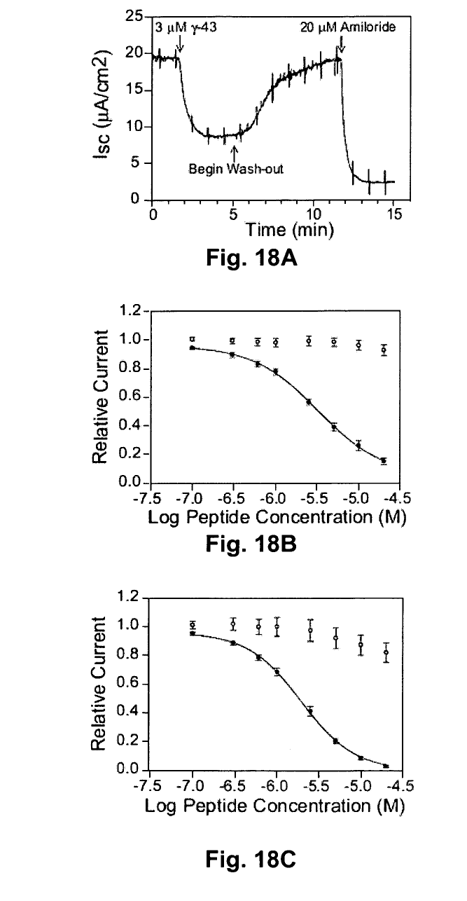

We next examined whether a synthetic peptide (γ-43) corresponding to the 43-mer tract excised from the γ subunit inhibited endogenous ENaC in epithelia. When added to the apical chamber of filter grown mouse cortical collecting duct cells (CCD), γ-43 (3 μM) rapidly inhibited short circuit currents (FIG. 18A). Peptide-dependent inhibition of the channel was reversible as the current was restored following washout of γ-43. This peptide inhibited short circuit currents in mouse CCD and in primary cultures of human airway epithelial (HAE) cells in a dose-dependent manner, with an IC₅₀ of 3.2 μM (CI, 2.7-3.8 μM) and 2.0 μM (CI, 1.9-2.2 μM), respectively (FIG. 18B and 18C) (n=8-9). In contrast, minimal effect on short circuit current was observed upon addition of a control, scrambled peptide. These data suggest that the 43-mer tract excised by furin- and prostasin-dependent proteolysis, functions as an endogenous inhibitor of ENaC activity prior to γ subunit cleavage.

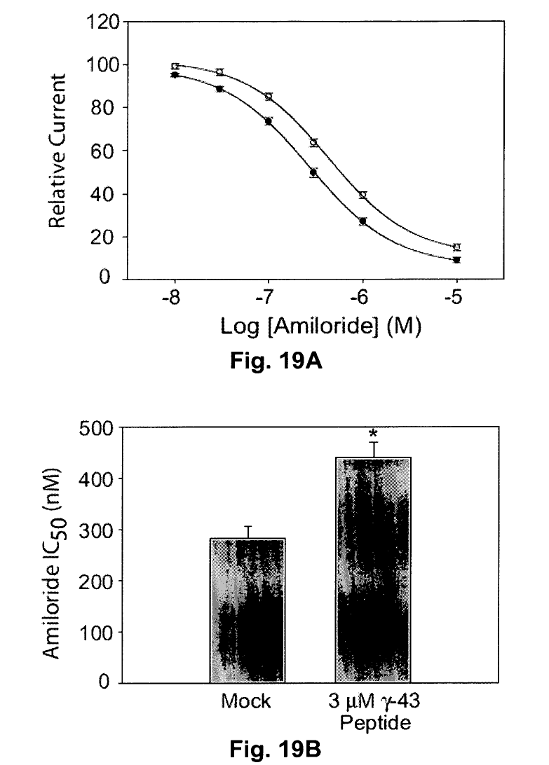

We recently reported that the inhibitory 26-mer peptide cleaved from the α subunit does not appear to interact with the amiloride binding site based on its lack of competition with amiloride (Carattino, M. D., et al. (2006) J. Biol. Chem. 281, 18901-18907). To our surprise, the γ-43 peptide significantly increased the IC₅₀ for amiloride block by 60% in CCD cells (FIG. 19). The amiloride IC₅₀ was 431 nM (CI, 339-548 nM) in the presence of 3 μM γ-43 and 270 nM (CI, 238-306 nM) in the absence of γ-43.

Discussion

We previously reported that ENaC α and γ subunits are processed by the endoprotease furin within their extracellular domains (Hughey, R. P., et al. (2004) J Biol Chem 279, 18111-18114). Two extracellular furin cleavage sites are present within the α subunit, and inhibition of furin dependent processing of the α subunit by mutating either one or both furin cleavage sites led to a profound inhibition of ENaC activity (Hughey, R. P., et al. (2004) J Biol Chem 279, 18111-18114; Sheng, S., et al. (2006) Am J Physiol Renal Physiol 290, F1488-1496 and Carattino, M. D., et al. (2006) J Biol. Chem. 281, 18901-18907). Furin dependent cleavage of the α subunit releases a 26-mer inhibitory peptide (Carattino, M. D., et al. (2006) J. Biol. Chem. 281, 18901-18907). We have also shown that the γ subunit is cleaved once by furin. In contrast to the α subunit, prevention of furin dependent processing of the γ subunit was associated with only a modest reduction in ENaC activity (Hughey, R. P., et al. (2004) J Biol Chem 279, 18111-18114).

Our observation that the α subunit must be cleaved twice to activate ENaC raised the possibility that the γ subunit also must be cleaved twice to fully activate ENaC by releasing a second inhibitory peptide. As there is only one furin cleavage site in the γ subunit, other proteases might be cleaving the γ subunit and activating ENaC. Our results suggest that prostasin, or α subsequent protease activated by prostasin, fulfills this task. Prostasin cleavage occurs immediately following tracts of basic amino acid residues (Shipway, A., et al. (2004), Biochemical characterization of prostasin, a channel activating protease, Biochem Biophys Res Commun 324, 953-963). We identified an RKRK¹⁸⁶ (SEQ ID NO: 7) tract ˜40 residues carboxyl-terminal to the furin cleavage site in γ as a potential prostasin-dependent cleavage site. Several lines of evidence suggest that prostasin is responsible for γ subunit cleavage at this site. Co-expression of ENaC and prostasin in MDCK cells resulted in the appearance of a second, more rapidly migrating γ subunit cleavage product consistent with the expected change in mobility upon cleavage at the RKRK¹⁸⁶ (SEQ ID NO: 7) site (FIGS. 14A and 20A). This second cleavage product was absent when prostasin was co-expressed with a mutant ENaC where the residues in the RKRK¹⁸⁶ (SEQ ID NO: 7) tract were changed to glutamine. Moreover, when this mutant was expressed in Xenopus oocytes, co-expression of prostasin failed to significantly activate ENaC, whereas this protease enhanced the activity of wild type ENaC (FIG. 14B). The fact that channels with the γ subunit RKRK¹⁸⁶ (SEQ ID NO: 7)/QQQQ (SEQ ID NO: 19) mutation exhibited lower currents than wild type channels suggests that there is limited proteolysis of wild type channels at this site in Xenopus oocytes in the absence of prostasin co-expression.

If prostasin and furin are activating ENaC as a result of two separate cleavage events in the γ subunit that releases a 43-mer inhibitory peptide, we reasoned that channels lacking this tract would exhibit an increase in channel activity. We found that channels lacking both this 43-mer tract and the furin cleavage site in the γ subunit exhibited greatly enhanced activity and an open probability that approached 1.0 (FIG. 17), even though the mutant γ subunit was not cleaved (FIG. 16B). The synthetic 43-mer peptide γ-43 reversibly inhibited endogenous ENaCs expressed in human airway epithelial cells and in mouse cortical collecting duct cells with IC₅₀'s of ˜2 to 3 μM (FIG. 18A-18C).

Although peptides excised from both the α and γ subunits inhibit ENaCs, they share limited similarity at the primary amino acid level (FIG. 13B). There are a number of other differences between these two inhibitory peptides. The 43-mer γ subunit derived peptide is a relatively potent inhibitor of ENaC expressed in human airway and mouse CCD cells (FIG. 18), in contrast to the 26-mer peptide (α-26) derived from α subunit cleavage that inhibited ENaC activity in human airway and mouse CCD cells with IC₅₀'s of 50 to 100 μM (Carattino, M. D., et al. (2006) J. Biol. Chem. 281, 18901-18907). The α subunit derived peptide did not alter the affect of amiloride on ENaC, suggesting that the α-26 peptide and amiloride interact at distinct sites within ENaC (Carattino, M. D., et al. (2006) J. Biol. Chem. 281, 18901-18907). In contrast, the γ subunit derived peptide γ-43 significantly altered the dose-dependent inhibition of amiloride (FIG. 19). While the γ-43 peptide and amiloride may interact at common sites within the channel, the modest change in the apparent amiloride IC₅₀ in the presence of γ-43 peptide is also consistent with amiloride and the γ-43 peptide interacting at distinct sites. Binding of the peptide to the channel may indirectly affect amiloride binding.

Previous studies demonstrating that the serine protease inhibitor aprotinin abolishes prostasin-induced activation of ENaC in Xenopus oocytes (Vuagniaux, G., et al. (2002) J Gen Physiol 120, 191-201; Vallet, V., et al. (1997) Nature 389, 607-610; Vuagniaux, G., et al. (2000) J Am Soc Nephrol 11, 828-834 and Adachi et al. (2001) J Am Soc Nephrol 12, 1114-1121) are consistent with the possibility that prostasin activates ENaC by cleaving the γ subunit.

Our results begin to address the question of why multiple proteases have a role in the activation of ENaC. Furin dependent cleavage of the α subunit at two sites releases a 26-mer inhibitory peptide (Carattino, M. D., et al. (2006) J. Biol. Chem. 281, 18901-18907). However, furin dependent cleavage of the γ subunit occurs at only one site and is not expected to release a γ subunit inhibitory peptide (Hughey, R. P., et al. (2004) J Biol Chem 279, 18111-18114). A second cleavage event mediated by prostasin or perhaps other proteases, is required to release a γ subunit inhibitory peptide. Harris et al. recently reported that exogenous neutrophil elastase cleaves the γ subunit of cell surface rat ENaC expressed in Xenopus oocytes and increases I_{Na} by 5- to 7-fold (Harris, M., Firsov, D., Vuagniaux, G., Stutts, M. J., and Rossier, B. C. (2006), A novel neutrophil elastase inhibitor prevents elastase activation and surface cleavage of the epithelial sodium channel expressed in Xenopus laevis oocytes, J Biol Chem, In Press).

We propose that multiple proteolytic cleavage events lead to a stepwise activation of ENaC, reflected in a stepwise increase in channel open probability. Channels that lack proteolytic processing have a low open probability (Sheng, S., et al. (2006) Am J Physiol Renal Physiol 290, F1488-1496. Channels that have been cleaved solely by furin exhibit an intermediate open probability, as these are likely the active channels that are observed in Xenopus oocytes at a single channel level (FIG. 15A) where there appears to be limited proteolytic release of the γ subunit inhibitory peptide under basal conditions. In contrast, channels that have released both α subunit and γ subunit inhibitory peptides exhibit a high open probability, as we observed in oocytes co-expressing ENaC and prostasin (FIG. 15B).

Previous studies in airway epithelial cells and renal cortical collecting duct cells have shown that non-specific inhibitors of prostasin, such as aprotinin, bikunin, and protease nexin-1 reduce ENaC activity (Vallet, V., et al. (1997) Nature 389, 607-610; Vuagniaux, G., et al. (2000) J Am Soc Nephrol 11, 828-834; Liu, L., Hering-Smith, K. S., Schiro, F. R., and Hamm, L. L. (2002), Serine protease activity in m-1 cortical collecting duct cells, Hypertension 39, 860-864; Adebamiro, A., Cheng, Y., Johnson, J. P., and Bridges, R. J. (2005), Endogenous protease activation of ENaC: effect of serine protease inhibition on ENaC single channel properties, J Gen. Physiol. 126, 339-352; Bridges, R. J., Newton, B. B., Pilewski, J. M., Devor, D. C., Poll, C. T., and Hall, R. L. (2001), Airway surface liquid volume regulates ENaC by altering the serine protease-protease inhibitor balance: a mechanism for sodium hyperabsorption in cystic fibrosis, Am J Physiol Lung Cell Mol Physiol 281, L16-23; Myerburg, M. M., Butterworth, M. B., McKenna, E. E., Peters, K. W., Frizzell, R. A., Kleyman, T. R., and Pilewski, J. M. (2006) J Biol Chem 281, 27942-27949; Adachi, M., et al. (2001) J Am Soc Nephrol 12, 1114-1121 and Wakida, N., Kitamura, K., Tuyen, D. G., Maekawa, A., Miyoshi, T., Adachi, M., Shiraishi, N., Ko, T., Ha, V., Nonoguchi, H., and Tomita, K. (2006), Inhibition of prostasin-induced ENaC activities by PN-1 and regulation of PN-1 expression by TGF-beta1 and aldosterone, Kidney Int 70, 1432-1438). Channel activity can be restored by either removing the inhibitor, or by treating cells with extracellular trypsin (Liu, L., Hering-Smith, K. S., Schiro, F. R., and Hamm, L. L. (2002) Hypertension 39, 860-864; Adebamiro, A., et al. (2005) J. Gen. Physiol. 126, 339-352 and Bridges, R. J., et al. (2001) Am J Physiol Lung Cell Mol Physiol 281, L16-23). In CCDs, we have also shown that a furin inhibitor dramatically reduced ENaC activity (Hughey, R. P., et al. (2004) J Biol Chem 279, 18111-18114). It has been unclear why inhibitors of both prostasin and furin reduce channel activity. We previously reported that two pools of channels are present at the plasma membrane, a pool of channels processed by furin and perhaps other proteases, and a second pool of near silent channels that has escaped proteolytic processing (Hughey, R. P., Bruns, J. B., Kinlough, C. L., and Kleyman, T. R. (2004) J Biol Chem 279, 48491-48494). We and others have suggested that these non-cleaved channels represent a pool of nearly silent channels and that prostasin or other proteases might activate ENaC by processing these non-cleaved channels at the plasma membrane Hughey, R. P., et al. (2004) J Biol Chem 279, 48491-48494; Caldwell, R. A., Boucher, R. C., and Stutts, M. J. (2004), Serine protease activation of near-silent epithelial Na+ channels, Am J Physiol Cell Physiol 286, C190-194 and Caldwell, R. A., Boucher, R. C., and Stutts, M. J. (2005), Neutrophil elastase activates near-silent epithelial Na+ channels and increases airway epithelial Na+ transport, Am J Physiol Lung Cell Mol Physiol 288, L813-819). Mechanisms by which prostasin activates ENaC are clearly more complex. In addition to the processing of non-cleaved channels, prostasin-dependent processing of channels that have been already cleaved by furin in the biosynthetic pathway further enhances channel activity.

In summary, our results suggest that prostasin activates ENaC by affecting cleavage of the γ subunit and, in concert with furin-dependent cleavage of the γ subunit, releases an inhibitory peptide. In a similar manner, furin-dependent processing within the α subunit at two sites releases an inhibitory peptide. These peptides released from the α and γ subunits may serve as initial templates for developing a new class of peptide based ENaC inhibitors.

Having now fully described this invention, it will be understood to those of ordinary skill in the art that the same can be performed within a wide and equivalent range of conditions, formulations and other parameters without affecting the scope of the invention or any embodiment thereof.

We claim:

1. An epithelial sodium channel-inhibitory agent comprising a polypeptide or polypeptide analog, other than a full length epithelial sodium channel γ-subunit, comprising the sequence:
X₁X₂X₃SX₄X₅X₆X₇X₈X₉GX₁₀X₁₁PX₁₂FX₁₃X₁₄X₁₅X₁₆PLX₁₇X₁₈FX₁₉X₂₀X₂₁X₂₂X₂₃X₂₄X₂₅ARDFX₂₆X₂₇X₂₈X₃₀X₂₉KRK (Formula II)(SEQ ID NO: 1)
wherein X₁₋₃₀ are, independently, any amino acid, X₃₀ may or may not be present, and wherein 0, 1, 2, 3, 4, 5, 6 or 7 of one or both of the N-terminal or C-terminal amino acids are not present.

2. The epithelial sodium channel-inhibitory agent of claim 1, comprising the sequence: EAESWSSX₇WEGTX₁₁PKFLNLIPLLVFNX₂₀DEKGKARDFFTGX₃₀RKRK (SEQ ID NO: 1, wherein residues 1=E, 2=A, 3=E, 5=W, 6=S, 7=S, 9=W, 10=E, 12=T, 15=K, 17=L, 18=N, 19=L, 20=I, 23=L, 24=V, 26=N, 28=D, 29=E, 30=K, 31=G, 32=K, 37=F, 38=T, 39=G and 41=R),
wherein X₇, X₁₁, X₂₀ and X₃₀ are, independently, any amino acid and X₃₀ may or may not be present.

3. The epithelial sodium channel-inhibitory agent of claim 2, wherein:
X₇ is T or V;
X₁₁ is P or Q;
X₂₀ is E or Q; and
X₃₀ is G or is not present.

4. The epithelial sodium channel-inhibitory agent of claim 2, wherein:
X₇ is T, V or A;
X₁₁ is P, Q, R or D;
X₂₀ is E, Q, K or R; and
X₃₀ is G or is not present.

5. The epithelial sodium channel-inhibitory agent of claim 2, wherein:
X₇ is T, V, A, S, P, G, N, D, E, Q, H, K, M, I, L or C;
X₁₁ is P, Q, R, D, T, S, N, E, K, M or H;
X₂₀ is E, Q, K, R, S, T, N, D, H or M; and
X₃₀ is G or is not present.

6. The epithelial sodium channel-inhibitory agent of claim 2, wherein:
X₇ is T, V, A, S, P, G, D, M, I, L;
X₁₁ is P, Q, R, D, T, E, K, N;
X₂₀ is E, Q, K, R, D; and
X₃₀ is G or is not present.

7. The epithelial sodium channel-inhibitory agent of claim 1, wherein the agent is a polypeptide.

8. The epithelial sodium channel-inhibitory agent of claim 1, in which 1, 2, 3, 4, 5, 6 or 7 of one or both of the N-terminal or C-terminal amino acids of Formula II are not present.

9. The epithelial sodium channel-inhibitory agent of claim 1, wherein the agent comprises the amino acid sequence: EAGSMRSTWEGTPPRFLNLIPLLVFNENEKGKARDFFTGRKRK (SEQ ID NO: 6, residues 144-186), EAESWNSVSEGKQPRFSHRIPLLIFDQDEKGKARDFFTGWKRK (SEQ ID NO: 5, residues 139-181), or EAX₃ SX₄X₅SX₇X₈EGX₁₀X₁₁PRFX₁₃X₁₄X₁₅IPLLX₁₈FX₁₉X₂₀X₂₁EKGKARDFFTGX₂₉KRK (SEQ ID NO: 20), where X₃ is G or E, X₄ is M or W, X₅ is R or N, X₇ is T or V, X₈ is W or S, X₁₀ is T or K, X₁i, is P or Q, X₁₃ is L or S, X₁₄ is N or H, X₁₅ is L or B, X₁₈ is V or I, X₁₉ is N or D, X₂₀ is E or Q, X₂₁ is N or D, X₂₉ is K or W and X₃₀ is not present or is any amino acid.

10. The epithelial sodium channel-inhibitory agent of claim 1, wherein X₁₋₃₀ are identified in the following table:

| | |
|---|---|
| X₁ | E |
| X₂ | A |
| X₃ | G, E |
| X₄ | M, W |
| X₅ | R, N |
| X₆ | S |
| X₇ | T, V |
| X₈ | W, S |
| X₉ | E |
| X₁₀ | T, K |
| X₁₁ | P, Q |
| X₁₂ | R |
| X₁₃ | L, S |
| X₁₄ | N, H |
| X₁₅ | L, R |
| X₁₆ | I |
| X₁₇ | L |
| X₁₈ | V, I |
| X₁₉ | N, D |
| X₂₀ | E, Q |
| X₂ₗ | N, D |
| X₂₂ | E |
| X₂₃ | K |
| X₂₄ | G |
| X₂₅ | K |
| X₂₆ | F |
| X₂₇ | T |
| X₂₈ | G |
| X₂₉ | R, W |
| X₃₀ | —. |

11. The epithelial sodium channel-inhibitory agent of claim 1, wherein X₁₋₃₀ are identified in the following table:

| | |
|---|---|
| X₁ | E, D |
| X₂ | A, T |
| X₃ | G, E, Q |
| X₄ | M, W |
| X₅ | R, N, P, S |
| X₆ | S, P |
| X₇ | T, V, A |
| X₈ | W, S, L, R |
| X₉ | E, K |
| X₁₀ | T, K, V |
| X₁₁ | P, Q, R, D |
| X₁₂ | R, K |
| X₁₃ | L, S, F |
| X₁₄ | N, H, K, R |
| X₁₅ | L, R |
| X₁₆ | I, V, A |
| X₁₇ | L, M |
| X₁₈ | V, I, A |
| X₁₉ | N, D, S, E |
| X₂₀ | E, Q, K, R |
| X₂₁ | N, D, G |
| X₂₂ | E, D |
| X₂₃ | K, T |
| X₂₄ | G, S |
| X₂₅ | K, Q |
| X₂₆ | F, L |
| X₂₇ | T, S |
| X₂₈ | G, L |
| X₂₉ | R, W |
| X₃₀ | — or G. |

12. The epithelial sodium channel-inhibitory agent of claim 1, wherein X₁₋₃₀ are identified in the following table:

| | |
|---|---|
| X₁ | E, D, S, T, N, Q, H, R, K |
| X₂ | A, T, C, S, G, P, N. D, E, Q, H, K |
| X₃ | G, E, Q, S, T, A, V, N, D, H, R, K, M |
| X₄ | M, W, Q, I, L, F, Y |
| X₅ | R, N, P, S, E, Q, H, K, T, G, D, K, A |
| X₆ | S, P, T, A, G, N, D, E, Q, K |
| X₇ | T, V, A, S, P, G, N, D, E, Q, H, K, M, I, L, C |
| X₈ | W, S, L, R, F, Y, T, A, G, N, D, E, Q, K, M, I, V, H |
| X₉ | E, K, S, T, N, D, Q, H, R |
| X₁₀ | T, K, V, S, P, G, N, D, E, Q, H, R, A, M, I, L |
| X₁₁ | P, Q, R, D, T, S, N, E, K, M, H |
| X₁₂ | R, K, N, E, Q, H, S, T |
| X₁₃ | L, S, F, M, I, V, A, G, N, D, E, Q, K, L, Y |
| X₁₄ | N, H, K, R, T, G, D, E, Q, N, S |
| X₁₅ | L, R, M, I, V, F, N, E, Q, H, K |
| X₁₆ | I, V, A, M, L, F, C, S, G |
| X₁₇ | L, M, I, V, F, Q |
| X₁₈ | V, I, A, M, L, F, C, S, G |
| X₁₉ | N, D, S, E, T, G, Q, R, K, A, H |
| X₂₀ | E, Q, K, R, S, T, N, D, H, M |
| X₂₁ | N, D, G, S, T, E, Q, R, K, A, V |
| X₂₂ | E, D, S, T, N, Q, H, R, K |
| X₂₃ | K, T, S, N, E, Q, R, P, G, D, H |
| X₂₄ | G, S, T, A, V, A, N, D, E, Q, K |
| X₂₅ | K, Q, S, T, N, E, R, D, H, M |
| X₂₆ | F, L, M, I, Y, W, V, F |
| X₂₇ | T, S, P, G, N, D, E, Q, H, K, |
| X₂₈ | G, L, S, T, A, V, M, I, F |
| X₂₉ | R, W, N, E, Q, H, K, F |
| X₃₀ | —, G, T, A, V. |

13. The epithelial sodium channel-inhibitory agent of claim 1, wherein X₁₋₃₀ are identified in the following table:

| | |
|---|---|
| X₁ | E, D, T, N, Q, K |
| X₂ | A, T, S, P, G, D |
| X₃ | G, E, Q, T, D, K |
| X₄ | M, W, I, L, F, Y |
| X₅ | R, N, P, S, Q, K, D, H, T, A |
| X₆ | S, P, T, A, N |
| X₇ | T, V, A, S, P, G, D, M, I, L |
| X₈ | W, S, L, R, F, Y, T, A, N, M, I, V, Q, K |
| X₉ | E, K, D, Q, R |
| X₁₀ | T, K, V, S, P, G, D, E, Q, R, M, I, L |
| X₁₁ | P, Q, R, D, T, E, K, N |
| X₁₂ | R, K, Q, E |
| X₁₃ | L, S, F, M, I, V, T, A, N, Y |
| X₁₄ | N, H, K, R, S, D, Y, E, Q |
| X₁₅ | L, R, M, I, V, Q, K |
| X₁₆ | I, V, A, M, L, S |
| X₁₇ | L, M, I, V |
| X₁₈ | V, I, A, M, L, S |
| X₁₉ | N, D, S, E, H, T, A, Q, K |
| X₂₀ | E, Q, K, R, D |
| X₂₁ | N, D, G, S, H, T, E |
| X₂₂ | E, D, T, Q, K, N |
| X₂₃ | K, T, E, Q, R, S, P, G, D |
| X₂₄ | G, S, T, A, N |
| X₂₅ | K, Q, E, R, K |
| X₂₆ | F, L, Y, M, I, V |
| X₂₇ | T, S, P, G, D, A, N |
| X₂₈ | G, L, T, M, I, V |
| X₂₉ | R, W, Q, K, F, Y |
| X₃₀ | —, G, T. |

14. The epithelial sodium channel-inhibitory agent of claim 1, consisting of a sequence of less than about 50 amino acids.

15. The epithelial sodium channel-inhibitory agent of claim 14, consisting of a sequence of about 43 amino acids.

16. The epithelial sodium channel-inhibitory agent of claim 14, consisting of a sequence of 43 amino acids.

17. The epithelial sodium channel-inhibitory agent of claim 1, wherein the agent is circularized.

18. The epithelial sodium channel-inhibitory agent of claim 1, comprising one or more N-substituted glycine residues.

19. The epithelial sodium channel-inhibitory agent of claim 1, comprising an amino acid sequence chosen from:

| | |
|---|---|
| EAGSMRSTWEGTPPRFLNLIPLLVFNENEKGK | (SEQ ID NO: 6, |
| ARDFFTGRKRK; | residues 144-186) |
| EAESWNSVSEGKQPRFSHRIPLLIFDQDEKGK | (SEQ ID NO: 5, |
| ARDFFTGWKRK; | residues 139-181) |
| EAGSMPSTLEGTPPRFFKLIPLLVFNENEKGK | (SEQ ID NO: 11) |
| ARDFFTGRKRK; | |
| EAESWSSAWEGTRPKFLRLVPLMVFSQDETSQ | (SEQ ID NO: 12) |
| ARDFLTGRKRK; | |
| EAQSWSSVRKGTDPKFLNLAPLMAFEKGDTGK | (SEQ ID NO: 13) |
| ARDFFTGRKRK; | |
| and | |
| DTESWSPAWEGVRPKFLNLVPLLIFNRDEKGK | (SEQ ID NO: 14) |
| ARDFLSLGRKRK. | |

20. The epithelial sodium channel-inhibitory agent of claim 19, wherein 1, 2 or 3 residues of the agent are conservatively substituted.

21. The epithelial sodium channel-inhibitory agent of claim 1, consisting of less than about 100 amino acids.

22. An epithelial sodium channel-inhibitory agent other than a full length epithelial sodium channel α-subunit, comprising one or more polypeptide or polypeptide analogs comprising a sequence chosen from: DX₁X₂X₃X₄X₅PHPLQRLX₆X₇PX₈PX₉X₁₀X₁₁X₁₂RX₁₃X₁₄R (SEQ ID NO: 2); DLRGX₄LPHPLQRLRVPPPPX₁₀X₁₁ARX₁₃AR (SEQ ID NO: 2, wherein residues 2=L, 3=R, 4=G, 6=L, 14=R, 15=V, 17=P, 19=P, 22=A, and 25=A); PHPLQRL (SEQ ID NO: 2, residues 7-13); PHPLQRLX₆X₇PX₈P (SEQ ID NO: 2, residues 7-18); DX₁X₂X₃X₄X₅PHPLQRLX₆X₇PX₈P (SEQ ID NO: 2, residues 1-18); PHPLQRLX₆X₇PX₈PX₉X₁₀X₁₁X₁₂ (SEQ ID NO: 2, residues 7-22); or PHPLQRLX₆X₇PX₈PX₉X₁₀X₁₁X₁₂RX₁₃X₁₄R (SEQ ID NO: 2, residues 7-26), wherein X₁₋₁₄ are, independently any amino acid, wherein the agent is not a polypeptide consisting of the sequence DLRGALPHPLQRLRTPPPPNPARSAR (SEQ ID NO: 4, residues 206-231).

23. The epithelial sodium channel-inhibitory agent of claim 22, the agent having the amino acid sequence DLRGX₄LPHPLQRLRX₇PPPPX₁₀X₁₁ARX₁₃AR (SEQ ID NO: 2, wherein 2=L, 3=R, 4=G, 6=L, 14=R, 17=P, 19=P, 22=A, and 25=A), where X₄ is A or T, X₇ is T or V, X₁₀ is N or H, X₁₁ is P or G and X₁₃ is S or R, wherein the agent is not one of the polypeptides DLRGALPHPLQRLRTPPPPNPARSAR (SEQ ID NO: 4, residues 206-231) or DLRGTLPHPLQRLRVPPPPHGARRAR (SEQ ID NO: 3, residues 179-204).

24. The epithelial sodium channel-inhibitory agent of claim 23, the agent comprising an amino acid sequence chosen from:

| | |
|---|---|
| DLRGALPHPLQRLRTPPPPNPARSAR; | (SEQ ID NO: 4, |
| | residues 206-231) |
| DLRGTLPHPLQRLRVPPPPHGARRAR; | (SEQ ID NO: 3, |
| | residues 179-204) |
| DLLGAFPHPLQRLRTPPPPYSGRTAR; | (SEQ ID NO: 8) |
| DLREPLPHPLQRLPVPAPPHAARGVR; | (SEQ ID NO: 9) |
| and | |
| DVHPPLPHPLQRLRVPPPRLEARRAR. | (SEQ ID NO: 10) |

25. The epithelial sodium channel-inhibitory agent of claim 24, wherein one or more residues of the sequence is conservatively substituted.

26. The epithelial sodium channel-inhibitory agent of claim 22, wherein X₁₋₁₄ are identified in the following table:

| | |
|---|---|
| X₁ | L |
| X₂ | R |
| X₃ | G |
| X₄ | A, T |
| X₅ | L |
| X₆ | R |
| X₇ | T, V |
| X₈ | P |
| X₉ | P |
| X₁₀ | N, H |
| X₁₁ | P, G |
| X₁₂ | A |
| X₁₃ | S, R, |
| X₁₄ | A. |

27. The epithelial sodium channel-inhibitory agent of claim 22, wherein X₁₋₁₄ are identified in the following table:

| | |
|---|---|
| X₁ | L, V |
| X₂ | R, L, H |
| X₃ | G, E, P |
| X₄ | A, T, P |
| X₅ | L, F |
| X₆ | R, P |
| X₇ | T, V |
| X₈ | P, A |
| X₉ | P, R |
| X₁₀ | N, H, Y, L |
| X₁₁ | P, G, S, A, E |
| X₁₂ | A, G |
| X₁₃ | S, R, T, G |
| X₁₄ | A, V. |

28. The epithelial sodium channel-inhibitory agent of claim 22, wherein X₁₋₁₄ are identified in the following table:

| | |
|---|---|
| X₁ | L, V, M, I, F |
| X₂ | R, L, H, N, E, Q, K, M, I, V, F, T, D, Y |
| X₃ | G, E, P, S, T, A, V, N, D, Q, H, R, K |
| X₄ | A, T, P, C, S, G, N, D, E, Q, H, K, |
| X₅ | L, F, M, I, V, Y, W |
| X₆ | R, P, N, E, Q, H, K, T |
| X₇ | T, V, S, P, G, N, D, E, Q, H, K, A, M, I, L, |
| X₈ | P, A, T, C, S, G, |
| X₉ | P, R, T, N, E, Q, H, K |
| X₁₀ | N, H, Y, L, S, G, D, E, Q, R, K, T, F, Y, W, M, I, V |
| X₁₁ | P, G, S, A, E, T, V, N, D, Q, K, C, H, R |
| X₁₂ | A, G, C, S, T, V |
| X₁₃ | S, R, T, G, A, N, D, E, Q, K, H, P, V |
| X₁₄ | A, V, C, S, G, M, I, L. |

29. The epithelial sodium channel-inhibitory agent of claim 22, wherein X₁₋₁₄ are identified in the following table:

| | |
|---|---|
| X₁ | L, V, M, I |
| X₂ | R, L, H, Q, K, M, I, V, N, Y |
| X₃ | G, E, P, T, D, Q, K |
| X₄ | A, P, S, G, D |
| X₅ | L, F, M, I, V, Y, W |
| X₆ | R, P, Q, K, T |
| X₇ | T, V, S, P, G, D, M, I, L |
| X₈ | P, A, T, S |
| X₉ | P, R, T, Q, K |
| X₁₀ | N, H, Y, L, S, D, F, W, M, I, V |
| X₁₁ | P, G, S, A, E, T, N, D, Q, K |
| X₁₂ | A, G, S, T |
| X₁₃ | S, R, T, G, A, N, Q, K, P, D |
| X₁₄ | A, V, S, M, I, L. |

30. The epithelial sodium channel-inhibitory agent of claim 22, the agent consisting of a sequence of less than about 35 amino acids.

31. The epithelial sodium channel-inhibitory agent of claim 30, the agent consisting of a sequence of about 26 amino acids.

32. The epithelial sodium channel-inhibitory agent of claim 30, the agent consisting of a sequence of 26 amino acids.

33. The epithelial sodium channel-inhibitory agent of claim 22, wherein the agent is a polypeptide.

34. The epithelial sodium channel-inhibitory agent of claim 22, wherein the agent is circularized.

35. The epithelial sodium channel-inhibitory agent of claim 22, the agent comprising one or more N-substituted glycine residues.

36. The epithelial sodium channel-inhibitory agent of claim 22, consisting of less than about 100 amino acids.

37. The epithelial sodium channel-inhibitory agent of claim 22, the agent comprising an amino acid sequence LPHPLQRL (SEQ ID NO: 2, residues 6-13).

38. A composition comprising an epithelial sodium channel-inhibitory agent, the agent comprising an amount of the agent effective to inhibit epithelial sodium channel activity in a patient, wherein the agent is a polypeptide or polypeptide analog comprising the sequence:
X₁X₂X₃SX₄X₅X₆X₇X₈X₉GX₁₀X₁₁PX₁₂FX₁₃X₁₄X₁₅X₁₆PLX₁₇X₁₈FX₁₉X₂₀X₂₁X₂₂X₂₃X₂₄X₂₅ARDFX₂₆X₂₇X₂₈X₃₀X₂₉KRK(Formula II)(SEQ ID NO: 1)
wherein X₁₋₃₀ are, independently, any amino acid, X₃₀ may or may not be present, and wherein 0, 1, 2, 3, 4, 5, 6 or 7 of one or both of the N-terminal or C-terminal amino acids are not present, and a pharmaceutically acceptable excipient.

39. The composition of claim 38, the agent comprising the sequence: EAESWSSX₇WEGTX₁₁PKFLNLIPLLVFNX₂₀DEKGKARDFFTGX₃₀RKRK (SEQ ID NO: 1, wherein residues I=E, 2=A, 3=E, 5=W, 6=S, 7=S, 9=W, 10=E, 12=T, 15=K, 17=L, 18=N, 19=L, 20=I, 23=L, 24=V, 26=N, 28=D, 29=E, 30=K, 31=G, 32=K, 37=F, 38=T, 39=G and 41=R) wherein X₇, X₁₁, X₂₀ and X₃₀ are, independently, any amino acid and X₃₀ may or may not be present.

40. The composition of claim 39, wherein:
X₇ is T or V;
X₁₁ is P or Q;
X₂₀ is E or Q; and
X₃₀ is G or is not present.

41. The composition of claim 39, wherein:
X₇ is T,V or A;
X₁₁ is P, Q, R or D;
X₂₀ is E, Q, K or R; and
X₃₀ is G or is not present.

42. The composition of claim 39, wherein:
X₇ is T, V, A, S, P, G, N, D, E, Q, H, K, M, I, L or C;
X₁₁ is P, Q, R, D, T, S, N, E, K, M or H;
X₂₀ is E, Q, K, R, S, T, N, D, H or M; and
X₃₀ is G or is not present.

43. The composition of claim 39, wherein:
X₇ is T, V, A, S, P, G, D, M, I, L;
X₁₁ is P, Q, R, D, T, E, K, N;
X₂₀ is E, Q, K, R, D; and
X₃₀ is G or is not present.

44. The composition of claim 3 8, wherein the agent is a polypeptide.

45. The composition of claim 38, in which 1, 2, 3, 4, 5, 6 or 7 of one or both of the N-terminal or C-terminal amino acids of Formula II are not present.

46. The composition of claim 38, wherein the agent comprises the amino acid sequence: EAGSMRSTWEGTPPRFLNLIPLLVFNENEKGKARDFFTGRKRK (SEQ ID NO: 6, residues 144-186), EAESWNSVSEGKQPRFSHRIPLLIFDQDEKGKARDFFTGWKRK (SEQ ID NO: 5, residues 139-181), or EAX₃SX₄X₅SX₇X₈EGX₁₀X₁₁PRFX₁₃X₁₄X₁₅IPLLX₁₈FX₁₉X₂₀X₂₁EKGKARDFFTGX₂₉KRK (SEQ ID NO: 20), where X₃ is G or E, X₄ is M or W, X₅ is R or N, X₇ is T or V, X₈ is W or S, X₁₀ is T or K, X₁₁ is P or Q, X₁₃ is L or S, X₁₄ is N or H, X₁₅ is L or B, X₁₈ is V or I, X₁₉ is N or D, X₂₀ is E or Q, X₂₁ is N or D, X₂₉ is K or W and X₃₀ is not present or is any amino acid.

47. The composition of claim 38, wherein X₁₋₃₀ are identified in the following table:

| | |
|---|---|
| X₁ | E |
| X₂ | A |
| X₃ | G, E |
| X₄ | M, W |
| X₅ | R, N |
| X₆ | S |
| X₇ | T, V |
| X₈ | W, S |
| X₉ | E |
| X₁₀ | T, K |
| X₁₁ | P, Q |
| X₁₂ | R |
| X₁₃ | L, S |
| X₁₄ | N, H |
| X₁₅ | L, R |
| X₁₆ | I |
| X₁₇ | L |
| X₁₈ | V, I |
| X₁₉ | N, D |
| X₂₀ | E, Q |
| X₂₁ | N, D |
| X₂₂ | E |
| X₂₃ | K |
| X₂₄ | G |
| X₂₅ | K |
| X₂₆ | F |
| X₂₇ | T |
| X₂₈ | G |
| X₂₉ | R, W |
| X₃₀ | —. |

48. The composition of claim 38, wherein X₁₋₃₀ are identified in the following table:

| | |
|---|---|
| X₁ | E, D |
| X₂ | A, T |
| X₃ | G, E, Q |
| X₄ | M, W |
| X₅ | R, N, P, S |
| X₆ | S, P |
| X₇ | T, V, A |
| X₈ | W, S, L, R |
| X₉ | E, K |
| X₁₀ | T, K, V |
| X₁₁ | P, Q, R, D |
| X₁₂ | R, K |
| X₁₃ | L, S, F |
| X₁₄ | N, H, K, R |
| X₁₅ | L, R |
| X₁₆ | I, V, A |
| X₁₇ | L, M |
| X₁₈ | V, I, A |
| X₁₉ | N, D, S, E |
| X₂₀ | E, Q, K, R |
| X₂₁ | N, D, G |
| X₂₂ | E, D |
| X₂₃ | K, T |
| X₂₄ | G, S |
| X₂₅ | K, Q |
| X₂₆ | F, L |
| X₂₇ | T, S |
| X₂₈ | G, L |
| X₂₉ | R, W |
| X₃₀ | — or G. |

49. The composition of claim 38, wherein X₁₋₃₀ are identified in the following table:

| | |
|---|---|
| X₁ | E, D, S, T, N, Q, H, R, K |
| X₂ | A, T, C, S, G, P, N. D, E, Q, H, K |
| X₃ | G, E, Q, S, T, A, V, N, D, H, R, K, M |
| X₄ | M, W, Q, I, L, F, Y |
| X₅ | R, N, P, S, E, Q, H, K, T, G, D, K, A |
| X₆ | S, P, T, A, G, N, D, E, Q, K |
| X₇ | T, V, A, S, P, G, N, D, E, Q, H, K, M, I, L, C |
| X₈ | W, S, L, R, F, Y, T, A, G, N, D, E, Q, |
| | K, M, I, V, H |
| X₉ | E, K, S, T, N, D, Q, H, R |
| X₁₀ | T, K, V, S, P, G, N, D, E, Q, H, R, A, M, I, L |
| X₁₁ | P, Q, R, D, T, S, N, E, K, M, H |
| X₁₂ | R, K, N, E, Q, H, S, T |
| X₁₃ | L, S, F, M, I, V, A, G, N, D, E, Q, K, L, Y |
| X₁₄ | N, H, K, R, T, G, D, E, Q, N, S |
| X₁₅ | L, R, M, I, V, F, N, E, Q, H, K |
| X₁₆ | I, V, A, M, L, F, C, S, G |
| X₁₇ | L, M, I, V, F, Q |
| X₁₈ | V, I, A, M, L, F, C, S, G |
| X₁₉ | N, D, S, E, T, G, Q, R, K, A, H |
| X₂₀ | E, Q, K, R, S, T, N, D, H, M |
| X₂₁ | N, D, G, S, T, E, Q, R, K, A, V |
| X₂₂ | E, D, S, T, N, Q, H, R, K |
| X₂₃ | K, T, S, N, E, Q, R, P, G, D, H |
| X₂₄ | G, S, T, A, V, A, N, D, E, Q, K |
| X₂₅ | K, Q, S, T, N, E, R, D, H, M |
| X₂₆ | F, L, M, I, Y, W, V, F |
| X₂₇ | T, S, P, G, N, D, E, Q, H, K, |
| X₂₈ | G, L, S, T, A, V, M, I, F |
| X₂₉ | R, W, N, E, Q, H, K, F |
| X₃₀ | —, G, T, A, V. |

50. The composition of claim 38, wherein X₁₋₃₀ are identified in the following table:

| | |
|---|---|
| X₁ | E, D, T, N, Q, K |
| X₂ | A, T, S, P, G, D |
| X₃ | G, E, Q, T, D, K |
| X₄ | M, W, I, L, F, Y |
| X₅ | R, N, P, S, Q, K, D, H, T, A |
| X₆ | S, P, T, A, N |
| X₇ | T, V, A, S, P, G, D, M, I, L |
| X₈ | W, S, L, R, F, Y, T, A, N, M, I, V, Q, K |
| X₉ | E, K, D, Q, R |
| X₁₀ | T, K, V, S, P, G, D, E, Q, R, M, I, L |
| X₁₁ | P, Q, R, D, T, E, K, N |
| X₁₂ | R, K, Q, E |
| X₁₃ | L, S, F, M, I, V, T, A, N, Y |
| X₁₄ | N, H, K, R, S, D, Y, E, Q |
| X₁₅ | L, R, M, I, V, Q, K |
| X₁₆ | I, V, A, M, L, S |
| X₁₇ | L, M, I, V |
| X₁₈ | V, I, A, M, L, S |
| X₁₉ | N, D, S, E, H, T, A, Q, K |
| X₂₀ | E, Q, K, R, D |
| X₂₁ | N, D, G, S, H, T, E |
| X₂₂ | E, D, T, Q, K, N |
| X₂₃ | K, T, E, Q, R, S, P, G, D |
| X₂₄ | G, S, T, A, N |
| X₂₅ | K, Q, E, R, K |
| X₂₆ | F, L, Y, M, I, V |
| X₂₇ | T, S, P, G, D, A, N |
| X₂₈ | G, L, T, M, I, V |
| X₂₉ | R, W, Q, K, F, Y |
| X₃₀ | —, G, T. |

51. The composition of claim 38, consisting of a sequence of less than about 50 amino acids.

52. The composition of claim 51, the agent consisting of a sequence of about 43 amino acids.

53. The composition of claim 51, the agent consisting of a sequence of 43 amino acids.

54. The composition of claim 38, wherein the agent is circularized.

55. The composition of claim 38, the agent comprising one or more N-substituted glycine residues.

56. The composition of claim 38, the agent comprising an amino acid sequence chosen from:

| | |
|---|---|
| EAGSMRSTWEGTPPRFLNLIPLLVFNENEKGK | (SEQ ID NO: 6, |
| ARDFFTGRKRK; | residues 144-186) |
| EAESWNSVSEGKQPRFSHRIPLLIFDQDEKGK | (SEQ ID NO: 5, |
| ARDFFTGWKRK; | residues 139-181) |
| EAGSMPSTLEGTPPRFFKLIPLLVFNFNEKGK | (SEQ ID NO: 11) |
| ARDFFTGRKRK; | |
| EAESWSSAWEGTRPKFLRLVPLMVFSQDETSQ | (SEQ ID NO: 12) |
| ARDFLTGRKRK; | |
| EAQSWSSVRKGTDPKFLNLAPLMAFEKGDTGK | (SEQ ID NO: 13) |
| ARDFFTGRKRK; | |
| and | |
| DTESWSPAWEGVRPKFLNLVPLLIFNRDEKGK | (SEQ ID NO: 14) |
| ARDFLSLGRKRK. | |

57. The composition of claim 56, in which any 1, 2 or 3 amino acids are conservatively substituted with another amino acid.

58. The composition of claim 38, comprising from about 100 pg to about 100 mg of the agent per unit dose.

59. The composition of claim 38, in which the composition is an aerosol or spray dosage form.

60. A composition comprising an epithelial sodium channel-inhibitory agent, the agent comprising an amount of the agent effective to inhibit epithelial sodium channel activity in a patient, wherein the agent is one or more polypeptide or polypeptide analog comprising a sequence: DX₁X₂X₃X₄X₅PHPLQRLX₆X₇PX₈PX₉X₁₀X₁₁X₁₂RX₁₃X₁₄R (SEQ ID NO: 2); DLRGX₄LPHPLQRLRVPPPPX₁₀X₁₁ARX₁₃AR (SEQ ID NO: 2, wherein residues 2=L, 3=R, 4=G, 6=L, 14=R, 15=V, 17=P, 19=P, 22=A, and 25=A); PHPLQRL (SEQ ID NO: 2, residues 7-13); PHPLQRLX₆X₇PX₈P (SEQ ID NO: 2, residues 7-18); DX₁X₂X₃X₄X₅PHPLQRLX₆X₇PX₈P (SEQ ID NO: 2, residues 1-18); PHPLQRLX₆X₇PX₈PX₉X₁₀X₁₁X₁₂ (SEQ ID NO: 2, residues 7-22); or PHPLQRLX₆X₇PX₈PX₉X₁₀X₁₁X₁₂RX₁₃X₁₄R (SEQ ID NO: 2, residues 7-26), wherein X₁₋₁₄ are, independently any amino acid, and a pharmaceutically acceptable excipient.

61. The composition of claim 60, the agent comprising one or more of the sequences DLRGALPHPLQRLRTPPPPNPARSAR(SEQ ID NO: 4, residues 206-231), DLRGTLPHPLQRLRVPPPPHGARRAR (SEQ ID NO: 3, residues 179-204), or DLRGX₄LPHPLQRLRX₇PPPPX₁₀X₁₁ARX₁₃AR (SEQ ID NO: 2, wherein 2=L, 3=R, 4=G, 6=L, 14=R, 17=P, 19=P, 22=A, and 25=A), where X₄ is A or T, X₇ is T or V, X₁₀ is N or H, X₁₁ is P or G and X₁₃ is S or R.

62. The composition of claim 60, the agent comprising an amino acid sequence chosen from:
DLRGALPHPLQRLRTPPPPNPARSAR (SEQ ID NO: 4, residues 206-231);
DLRGTLPHPLQRLRVPPPPHGARRAR (SEQ ID NO: 3, residues 179-204);
DLLGAFPHPLQRLRTPPPPYSGRTAR (SEQ ID NO: 8);
DLREPLPHPLQRLPVPAPPHAARGVR (SEQ ID NO: 9); and
DVHPPLPHPLQRLRVPPPRLEARRAR (SEQ ID NO: 10).

63. The composition of claim 62, wherein one or more residues of the agent are conservatively substituted.

64. The composition of claim 60, wherein X₁₋₁₄ are identified in the following table:

| | |
|---|---|
| X₁ | L |
| X₂ | R |
| X₃ | G |
| X₄ | A, T |
| X₅ | L |
| X₆ | R |
| X₇ | T, V |
| X₈ | P |
| X₉ | P |
| X₁₀ | N, H |
| X₁₁ | P, G |
| X₁₂ | A |
| X₁₃ | S, R, |
| X₁₄ | A. |

65. The composition of claim 60, wherein X₁₋₁₄ are identified in the following table:

| | |
|---|---|
| X₁ | L, V |
| X₂ | R, L, H |
| X₃ | G, E, P |
| X₄ | A, T, P |
| X₅ | L, F |
| X₆ | R, P |
| X₇ | T, V |
| X₈ | P, A |
| X₉ | P, R |
| X₁₀ | N, H, Y, L |
| X₁₁ | P, G, S, A, E |
| X₁₂ | A, G |
| X₁₃ | S, R, T, G |
| X₁₄ | A, V. |

66. The composition of claim 60, wherein X₁₋₁₄ are identified in the following table:

| | |
|---|---|
| X₁ | L, V, M, I, F |
| X₂ | R, L, H, N, E, Q, K, M, I, V, F, T, D, Y |
| X₃ | G, E, P, S, T, A, V, N, D, Q, H, R, K |
| X₄ | A, T, P, C, S, G, N, D, E, Q, H, K, |
| X₅ | L, F, M, I, V, Y, W |
| X₆ | R, P, N, E, Q, H, K, T |
| X₇ | T, V, S, P, G, N, D, E, Q, H, K, A, M, I, L, |
| X₈ | P, A, T, C, S, G, |
| X₉ | P, R, T, N, E, Q, H, K |
| X₁₀ | N, H, Y, L, S, G, D, E, Q, R, K, T, F, Y, W, M, I, V |
| X₁₁ | P, G, S, A, E, T, V, N, D, Q, K, C, H, R |
| X₁₂ | A, G, C, S, T, V |
| X₁₃ | S, R, T, G, A, N, D, E, Q, K, H, P, V |
| X₁₄ | A, V, C, S, G, M, I, L. |

67. The composition of claim 60, wherein X₁₋₁₄ are identified in the following table:

| | |
|---|---|
| X₁ | L, V, M, I |
| X₂ | R, L, H, Q, K, M, I, V, N, Y |
| X₃ | G, E, P, T, D, Q, K |
| X₄ | A, P, S, G, D |
| X₅ | L, F, M, I, V, Y, W |
| X₆ | R, P, Q, K, T |
| X₇ | T, V, S, P, G, D, M, I, L |
| X₈ | P, A, T, S |
| X₉ | P, R, T, Q, K |
| X₁₀ | N, H, Y, L, S, D, F, W, M, I, V |
| X₁₁ | P, G, S, A, E, T, N, D, Q, K |
| X₁₂ | A, G, S, T |
| X₁₃ | S, R, T, G, A, N, Q, K, P, D |
| X₁₄ | A, V, S, M, I, L. |

68. The composition of claim 60, the agent consisting of a sequence of less than about 35 amino acids.

69. The composition of claim 68, the agent consisting of a sequence of about 26 amino acids.

70. The composition of claim 68, the agent consisting of a sequence of 26 amino acids.

71. The composition of claim 60, wherein the agent is a polypeptide.

72. The composition of claim 60, wherein the agent is circularized.

73. The composition of claim 60, the agent comprising one or more N-substituted glycine residues.

74. The epithelial sodium channel-inhibitory agent of claim 60, in which the sequence is DX₁X₂X₃X₄X₅PHPLQRLX₆X₇PX₈PX₉X₁₀X₁₁X₁₂RX₁₃X₁₄R (SEQ ID NO: 2, wherein 2=L, 3=R, 4=G, 6=L, 14=R, 17=P, 19=P, 22=A, and 25=A) and 1, 2, 3, 4, 5, 6 or 7 of one or both of the N-terminal or C-terminal amino acids of the sequence are not present.

75. The composition of claim 60, comprising from about 100 pg to about 100 mg of the agent per unit dose.

76. The composition of claim 60, in which the composition is an aerosol or spray dosage form.

77. The composition of claim 60, the agent comprising an amino acid sequence LPHPLQRL (SEQ ID NO: 2, residues 6-13).

78. A method of characterizing the activity of an epithelial sodium channel-inhibitory agent, comprising testing the epithelial sodium channel-inhibitory agent for inhibition of epithelial sodium channel activity, the agent comprising a sequence: DX₁X₂X₃X₄X₅PHPLQRLX₆X₇PX₈PX₉X₁₀X₁₁X₁₂RX₁₃X₁₄R (SEQ ID NO: 2); DLRGX₄LPHPLQRLRVPPPPX₁₀X₁₁ARX₁₃AR (SEQ ID NO: 2, wherein residues 2=L, 3=R, 4=G, 6=L, 14=R, 15=V, 17=P, 19=P, 22=A, and 25=A); PHPLQRL (SEQ ID NO: 2, residues 7-13); PHPLQRLX₆X₇PX₈P (SEQ ID NO: 2, residues 7-18); DX₁X₂X₃X₄X₅PHPLQRLX₆X₇PX₈P (SEQ ID NO: 2, residues 1-18); PHPLQRLX₆X₇PX₈PX₉X₁₀X₁₁X₁₂ (SEQ ID NO: 2, residues 7-22); or PHPLQRLX₆X₇PX₈PX₉X₁₀X₁₁X₁₂RX₁₃X₁₄R (SEQ ID NO: 2, residues 7-26), wherein X₁₋₁₄ are, independently, any amino acid.

79. The method of claim 78, the agent comprising one or more of the sequences DLRGALPHPLQRLRTPPPPNPARSAR (SEQ ID NO: 4, residues 206-231), DLRGTLPHPLQRLRVPPPPHGARRAR (SEQ ID NO: 3, residues 179-204), or DLRGX₄LPHPLQRLRX₇PPPPX₁₀X₁₁ARX₁₃AR (SEQ ID NO: 2, wherein 2=L, 3=R, 4=G, 6=L, 14=R, 17=P, 19=P, 22=A, and25=A),where X₄ is A or T, X₇ is T or V, X₁₀ is N or H, X₁₁ is P or G and X₁₃ is S or R.

80. The method of claim 78, wherein X₁₋₁₄ are identified in the following table:

| | |
|---|---|
| X₁ | L |
| X₂ | R |
| X₃ | G |
| X₄ | A, T |
| X₅ | L |
| X₆ | R |
| X₇ | T, V |
| X₈ | P |
| X₉ | P |
| X₁₀ | N, H |
| X₁₁ | P, G |
| X₁₂ | A |
| X₁₃ | S, R, |
| X₁₄ | A. |

81. The method of claim 78, wherein X₁₋₁₄ are identified in the following table:

| | |
|---|---|
| X₁ | L, V |
| X₂ | R, L, H |
| X₃ | G, E, P |
| X₄ | A, T, P |
| X₅ | L, F |
| X₆ | R, P |
| X₇ | T, V |
| X₈ | P, A |
| X₉ | P, R |
| X₁₀ | N, H, Y, L |
| X₁₁ | P, G, S, A, E |
| X₁₂ | A, G |
| X₁₃ | S, R, T, G |
| X₁₄ | A, V. |

82. The method of claim 78, wherein X₁₋₁₄ are identified in the following table:

| | |
|---|---|
| X₁ | L, V, M, I, F |
| X₂ | R, L, H, N, E, Q, K, M, I, V, F, T, D, Y |
| X₃ | G, E, P, S, T, A, V, N, D, Q, H, R, K |
| X₄ | A, T, P, C, S, G, N, D, E, Q, H, K, |
| X₅ | L, F, M, I, V, Y, W |
| X₆ | R, P, N, E, Q, H, K, T |
| X₇ | T, V, S, P, G, N, D, E, Q, H, K, A, M, I, L, |
| X₈ | P, A, T, C, S, G, |
| X₉ | P, R, T, N, E, Q, H, K |
| X₁₀ | N, H, Y, L, S, G, D, E, Q, R, K, T, F, Y, W, M, I, V |
| X₁₁ | P, G, S, A, E, T, V, N, D, Q, K, C, H, R |
| X₁₂ | A, G, C, S, T, V |
| X₁₃ | S, R, T, G, A, N, D, E, Q, K, H, P, V |
| X₁₄ | A, V, C, S, G, M, I, L. |

83. The method of claim 78, wherein X₁₋₁₄ are identified in the following table:

| | |
|---|---|
| X₁ | L, V, M, I |
| X₂ | R, L, H, Q, K, M, I, V, N, Y |
| X₃ | G, E, P, T, D, Q, K |
| X₄ | A, P, S, G, D |
| X₅ | L, F, M, I, V, Y, W |
| X₆ | R, P, Q, K, T |
| X₇ | T, V, S, P, G, D, M, I, L |
| X₈ | P, A, T, S |
| X₉ | P, R, T, Q, K |
| X₁₀ | N, H, Y, L, S, D, F, W, M, I, V |
| X₁₁ | P, G, S, A, E, T, N, D, Q, K |
| X₁₂ | A, G, S, T |
| X₁₃ | S, R, T, G, A, N, Q, K, P, D |
| X₁₄ | A, V, S, M, I, L. |

84. The method of claim 78, the agent consisting of a sequence of less than about 35 amino acids.

85. The method of claim 84, the agent consisting of a sequence of about 26 amino acids.

86. The method of claim 84, the agent consisting of a sequence of 26 amino acids.

87. The method of claim 78, wherein the agent is a polypeptide.

88. The method of claim 78, wherein the agent is circularized.

89. The method of claim 78, the agent comprising one or more N-substituted glycine residues.

90. The method of claim 78, the agent comprising an amino acid sequence chosen from:
DLRGALPHPLQRLRTPPPPNPARSAR (SEQ ID NO: 4, residues 206-231);
DLRGTLPHPLQRLRVPPPPHGARRAR (SEQ ID NO: 3, residues 179-204);
DLLGAFPHPLQRLRTPPPPYSGRTAR (SEQ ID NO: 8);
DLREPLPHPLQRLPVPAPPHAARGVR (SEQ ID NO: 9); and
DVHPPLPHPLQRLRVPPPRLEARRAR (SEQ ID NO: 10).

91. The method of claim 90, wherein one or more residues of the sequence is conservatively substituted.

92. The method of claim 90, in which any 1, 2 or 3 amino acids are conservatively substituted with another amino acid.

93. The method of claim 78, in which the sequence is: DX₁X₂X₃X₄X₅PHPLQRLX₆X₇PX₈PX₉X₁₀X₁₁X₁₂RX₁₃X₁₄R (SEQ ID NO: 2) and 1, 2, 3, 4, 5, 6 or 7 of one or both of the N-terminal or C-terminal amino acids of the sequence are not present.

94. The method of claim 78, the agent comprising an amino acid sequence LPHPLQRL (SEQ ID NO: 2, residues 6-13).

95. A method of characterizing the activity of an epithelial sodium channel-inhibitory agent, comprising testing the epithelial sodium channel-inhibitory agent for inhibition of epithelial sodium channel activity, the agent comprising a sequence:
X₁X₂X₃SX₄X₅X₆X₇X₈X₉GX₁₀X₁₁PX₁₂FX₁₃X₁₄X₁₅X₁₆PLX₁₇X₁₈FX₁₉X₂₀X₂₁X₂₂X₂₃X₂₄X₂₅ARDFX₂₆X₂₇X₂₈X₃₀X₂₉KRK (Formula II)(SEQ ID NO: 1)
wherein X₁₋₃₀ are, independently, any amino acid, X₃₀ may or may not be present, and wherein 0, 1, 2, 3, 4, 5, 6 or 7 of one or both of the N-terminal or C-terminal amino acids are not present.

96. The method of claim 95, comprising the sequence: EAESWSSX₇WEGTX₁₁PKFLNLIPLLVFNX₂₀DEKGKARDFFTGX₃₀RKRK (SEQ ID NO: 1, wherein residues 1=E, 2=A, 3=E, 5=W, 6=S, 7=S, 9=W, 10=E, 12=T, 15=K, 17=L, 18=N, 19=L, 20 =I, 23=L, 24=V, 26=N, 28=D, 29=E, 30=K, 31=G, 32=K, 37=F, 38=T, 39=G and 41=R) X₇, X₁₁, X₂₀ and X₃₀ are, independently, any amino acid and X₃₀ may or may not be present.

97. The method of claim 96, wherein:
X₇ is T or V;
X₁₁ is P or Q;
X₂₀ is E or Q; and
X₃₀ is G or is not present.

98. The method of claim 96, wherein:
X₇ is T, V or A;
X₁₁ is P, Q, R or D;
X₂₀ is E, Q, K or R; and
X₃₀ is G or is not present.

99. The method of claim 96, wherein:
X₇ is T, V, A, S, P, G, N, D, E, Q, H, K, M, I, L or C;
X₁₁ is P, Q, R, D, T, S, N, E, K, M or H;
X₂₀ is E, Q, K, R, S, T, N, D, H or M; and
X₃₀ is G or is not present.

100. The method of claim 96, wherein:
X₇ is T, V, A, S, P, G, D, M, I, L;
X₁₁ is P, Q, R, D, T, E, K, N;
X₂₀ is E, Q, K, R, D; and
X₃₀ is G or is not present.

101. The method of claim 95, wherein the agent is a polypeptide.

102. The method of claim 95, wherein the agent comprises one or more N-substituted glycine residues.

103. The method of claim 95, wherein the agent comprises the amino acid sequence: EAGSMRSTWEGTPPRFLNLIPLLVFNENEKGKARDFFTGRKRK (SEQ ID NO: 6, residues 144-186), EAESWNSVSEGKQPRFSHRIPLLIFDQDEKGKARDFFTGWKRK (SEQ ID NO: 5, residues 139-181), or EAX₃SX₄X₅SX₇X₈EGX₁₀X₁₁PRFX₁₃X₁₄X₁₅IPLLX₁₈FX₁₉X₂₀X₂₁EKGKARDFFTGX₂₉KRK (SEQ ID NO: 20), where X₃ is G or E, X₄ is M or W, X₅ is R or N, X₇ is T or V, X₈ is W or S, X₁₀ is T or K, X₁₁ is P or Q, X₁₃ is L or S, X₁₄ is N or H, X₁₅ is L or B, X₁₈ is V or I, X₁₉ is N or D, X₂₀ is E or Q, X₂₁ is N or D, X₂₉ is K or W and X₃₀ is not present or is any amino acid.

104. The method of claim 95, wherein X₁₋₃₀ are identified in the following table:

| | |
|---|---|
| X₁ | E |
| X₂ | A |
| X₃ | G, E |
| X₄ | M, W |
| X₅ | R, N |
| X₆ | S |
| X₇ | T, V |
| X₈ | W, S |
| X₉ | E |
| X₁₀ | T, K |
| X₁₁ | P, Q |
| X₁₂ | R |
| X₁₃ | L, S |
| X₁₄ | N, H |
| X₁₅ | L, R |
| X₁₆ | I |
| X₁₇ | L |
| X₁₈ | V, I |
| X₁₉ | N, D |
| X₂₀ | E, Q |
| X₂₁ | N, D |
| X₂₂ | E |
| X₂₃ | K |
| X₂₄ | G |
| X₂₅ | K |
| X₂₆ | F |
| X₂₇ | T |
| X₂₈ | G |
| X₂₉ | R, W |
| X₃₀ | —. |

105. The method of claim 95, wherein X₁₋₃₀ are identified in the following table:

| | |
|---|---|
| X₁ | E, D |
| X₂ | A, T |
| X₃ | G, E, Q |
| X4 | M, W |
| X₅ | R, N, P, S |
| X₆ | S, P |
| X₇ | T, V, A |
| X₈ | W, S, L, R |
| X₉ | E, K |
| X₁₀ | T, K, V |
| X₁₁ | P, Q, R, D |
| X₁₂ | R, K |
| X₁₃ | L, S, F |
| X₁₄ | N, H, K, R |
| X₁₅ | L, R |
| X₁₆ | I, V, A |
| X₁₇ | L, M |
| X₁₈ | V, I, A |
| X₁₉ | N, D, S, E |
| X₂₀ | E, Q, K, R |
| X₂₁ | N, D, G |
| X₂₂ | E, D |
| X₂₃ | K, T |
| X₂₄ | G, S |
| X₂₅ | K, Q |
| X₂₆ | F, L |
| X₂₇ | T, S |
| X₂₈ | G, L |
| X₂₉ | R, W |
| X₃₀ | — or G. |

106. The method of claim 95, wherein X₁₋₃₀ are identified in the following table:

| | |
|---|---|
| X₁ | E, D, S, T, N, Q, H, R, K |
| X₂ | A, T, C, S, G, P, N. D, E, Q, H, K |
| X₃ | G, E, Q, S, T, A, V, N, D, H, R, K, M |
| X₄ | M, W, Q, I, L, F, Y |
| X₅ | R, N, P, S, E, Q, H, K, T, G, D, K, A |
| X₆ | S, P, T, A, G, N, D, E, Q, K |
| X₇ | T, V, A, S, P, G, N, D, E, Q, H, K, M, I, L, C |
| X₈ | W, S, L, R, F, Y, T, A, G, N, D, E, Q, |
| | K, M, I, V, H |
| X₉ | E, K, S, T, N, D, Q, H, R |
| X₁₀ | T, K, V, S, P, G, N, D, E, Q, H, R, A, M, I, L |
| X₁₁ | P, Q, R, D, T, S, N, E, K, M, H |
| X₁₂ | R, K, N, E, Q, H, S, T |
| X₁₃ | L, S, F, M, I, V, A, G, N, D, E, Q, K, L, Y |
| X₁₄ | N, H, K, R, T, G, D, E, Q, N, S |
| X₁₅ | L, R, M, I, V, F, N, E, Q, H, K |
| X₁₆ | I, V, A, M, L, F, C, S, G |
| X₁₇ | L, M, I, V, F, Q |
| X₁₈ | V, I, A, M, L, F, C, S, G |
| X₁₉ | N, D, S, E, T, G, Q, R, K, A, H |
| X₂₀ | E, Q, K, R, S, T, N, D, H, M |
| X₂₁ | N, D, G, S, T, E, Q, R, K, A, V |
| X₂₂ | E, D, S, T, N, Q, H, R, K |
| X₂₃ | K, T, S, N, E, Q, R, P, G, D, H |
| X₂₄ | G, S, T, A, V, A, N, D, E, Q, K |
| X₂₅ | K, Q, S, T, N, E, R, D, H, M |
| X₂₆ | F, L, M, I, Y, W, V, F |
| X₂₇ | T, S, P, G, N, D, E, Q, H, K, |
| X₂₈ | G, L, S, T, A, V, M, I, F |
| X₂₉ | R, W, N, E, Q, H, K, F |
| X₃₀ | —, G, T, A, V. |

107. The method of claim 95, wherein X₁₋₃₀ are identified in the following table:

| | |
|---|---|
| X₁ | E, D, T, N, Q, K |
| X₂ | A, T, S, P, G, D |
| X₃ | G, E, Q, T, D, K |
| X₄ | M, W, I, L, F, Y |
| X₅ | R, N, P, S, Q, K, D, H, T, A |
| X₆ | S, P, T, A, N |
| X₇ | T, V, A, S, P, G, D, M, I, L |
| X₈ | W, S, L, R, F, Y, T, A, N, M, I, V, Q, K |
| X₉ | E, K, D, Q, R |
| X₁₀ | T, K, V, S, P, G, D, E, Q, R, M, I, L |
| X₁₁ | P, Q, R, D, T, E, K, N |
| X₁₂ | R, K, Q, E |
| X₁₃ | L, S, F, M, I, V, T, A, N, Y |
| X₁₄ | N, H, K, R, S, D, Y, E, Q |
| X₁₅ | L, R, M, I, V, Q, K |
| X₁₆ | I, V, A, M, L, S |
| X₁₇ | L, M, I, V |
| X₁₈ | V, I, A, M, L, S |
| X₁₉ | N, D, S, E, H, T, A, Q, K |
| X₂₀ | E, Q, K, R, D |
| X₂₁ | N, D, G, S, H, T, E |
| X₂₂ | E, D, T, Q, K, N |
| X₂₃ | K, T, E, Q, R, S, P, G, D |
| X₂₄ | G, S, T, A, N |
| X₂₅ | K, Q, E, R, K |
| X₂₆ | F, L, Y, M, I, V |
| X₂₇ | T, S, P, G, D, A, N |
| X₂₈ | G, L, T, M, I, V |
| X₂₉ | R, W, Q, K, F, Y |
| X₃₀ | —, G, T. |

108. The method of claim 95, consisting of a sequence of less than about 50 amino acids.

109. The method of claim 108, consisting of a sequence of about 43 amino acids.

110. The method of claim 108, consisting of a sequence of 43 amino acids.

111. The method of claim 95, wherein the agent is circularized.

112. The method of claim 95, comprising an amino acid sequence chosen from:

| | |
|---|---|
| EAGSMRSTWEGTPPRFLNLIPLLVFNENEKGK | (SEQ ID NO: 6, |
| ARDFFTGRKRK; | residues 144-186) |
| EAESWNSVSEGKQPRFSHRIPLLIFDQDEKGK | (SEQ ID NO: 5, |
| ARDFFTGWKRK; | residues 139-181) |
| EAGSMPSTLEGTPPRFFKLIPLLVFNENEKGK | (SEQ ID NO: 11) |
| ARDFFTGRKRK; | |
| EAESWSSAWEGTRPKFLRLVPLMVFSQDETSQ | (SEQ ID NO: 12) |
| ARDFLTGRKRK; | |
| EAQSWSSVRKGTDPKFLNLAPLMAFEKGDTGK | (SEQ ID NO: 13) |
| ARDFFTGRKRK; | |
| and | |
| DTESWSPAWEGVRPKFLNLVPLLIFNRDEKGK | (SEQ ID NO: 14) |
| ARDFLSLGRKRK. | |

113. The method of claim 112, wherein one or more residues of the agent are conservatively substituted.

114. The method of claim 112, in which any 1, 2 or 3 amino acids are conservatively substituted with another amino acid.

115. A method of inhibiting epithelial sodium channel activity in cells of a patient's airway, comprising administering to the patient an amount of a composition claimed in claim 38, effective to inhibit activity of epithelial sodium channel in cells of the patient's airway, thereby inhibiting activity of the epithelial sodium channel in the patient's airway.

116. A method of inhibiting epithelial sodium channel activity in cells of a patient's airway, comprising administering to the patient an amount of a composition claimed in claim 60, effective to inhibit activity of epithelial sodium channel in cells of the patient's airway, thereby inhibiting activity of the epithelial sodium channel in the patient's airway.


SEQUENCE LISTING (20 sequences)

SEQ ID NO: 1 - protein, 44 aa, Artificial
  Generic ENaC gamma agonist
  MISC_FEATURE (1)..(41): Any X can be any amino acid
XXXSXXXXXXGXXPXFXXXXPLXXFXXXXXXXARDFXXXXXKRK

SEQ ID NO: 2 - protein, 26 aa, Artificial
  Generic ENaC alpha agonist
  MISC_FEATURE (2)..(25): Any X can be any amino acid
DXXXXXPHPLQRLXXPXPXXXXRXXR

SEQ ID NO: 3 - protein, 669 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 4 - protein, 699 aa, Mus musculus
MLDHTRAPELNLDLDLDVSNSPKGSMKGNNFKEQDLCPPLPMQGLGKGDKREEQALGPEPSEPRQPTEEEEALIEFHRSYRELFQFFCNNTTIHGAIRLVCSKHNRMKTAFWAVLWLCTFGMMYWQFALLFEEYFSYPVSLNINLNSDKLVFPAVTVCTLNPYRYTEIKEDLEELDRITEQTLFDLYKYNSSYTRQAGGRRRSTRDLRGALPHPLQRLRTPPPPNPARSARSASSSVRDNNPQVDRKDWKIGFQLCNQNKSDCFYQTYSSGVDAVREWYRFHYINILSRLPDTSPALEEEALGSFIFTCRFNQAPCNQANYSQFHHPMYGNCYTFNNKNNSNLWMSSMPGVNNGLSLTLRTEQNDFIPLLSTVTGARVMVHGQDEPAFMDDGGFNVRPGVETSISMRKEALDSLGGNYGDCTENGSDVPVKNLYPSKYTQQVCIHSCFQENMIKKCGCAYIFYPKPKGVEFCDYLKQSSWGYCYYKLQAAFSLDSLGCFSKCRKPCSVTNYKLSAGYSRWPSVKSQDWIFEMLSLQNNYTINNKRNGVAKLNIFFKELNYKTNSESPSVTMVSLLSNLGSQWSLWFGSSVLSVVEMAELIFDLLVITLIMLLHRFRSRYWSPGRGARGAREVASTPASSFPSRFCPHPTSPPPSLPQQGTTPPLALTAPPPAYATLGPSASPLDSAVPGSSACAPAMAL

SEQ ID NO: 5 - protein, 649 aa, Homo sapiens
MAPGEKIKAKIKKNLPVTGPQAPTIKELMRWYCLNTNTHGCRRIVVSRGRLRRLLWIGFTLTAVALILWQCALLVFSFYTVSVSIKVHFRKLDFPAVTICNINPYKYSTVRHLLADLEQETREALKSLYGFPESRKRREAESWNSVSEGKQPRFSHRIPLLIFDQDEKGKARDFFTGRKRKVGGSIIHKASNVMHIESKQVVGFQLCSNDTSDCATYTFSSGINAIQEWYKLHYMNIMAQVPLEKKINMSYSAEELLVTCFFDGVSCDARNFTLFHHPMHGNCYTFNNRENETILSTSMGGSEYGLQVILYINEEEYNPFLVSSTGAKVIIHRQDEYPFVEDVGTEIETAMVTSIGMHLTESFKLSEPYSQCTEDGSDVPIRNIYNAAYSLQICLHSCFQTKMVEKCGCAQYSQPLPPAANYCNYQQHPNWMYCYYQLHRAFVQEELGCQSVCKEACSFKEWTLTTSLAQWPSVVSEKWLLPVLTWDQGRQVNKKLNKTDLAKLLIFYKDLNQRSIMESPANSIEMLLSNFGGQLGLWMSCSVVCVIEIIEVFFIDFFSIIARRQWQKAKEWWAWKQAPPCPEAPRSPQGQDNPALDIDDDLPTFNSALHLPPALGTQVPGTPPPKYNTLRLERAFSNQLTDTQMLDEL

SEQ ID NO: 6 - protein, 655 aa, Mus musculus
MAPGEKIKAKIKKNLPVRGPQAPTIKDLMHWYCLNTNTHGCRRIVVSRGRLRRLLWIAFTLTAVALIIWQCALLVFSFYTVSVSIKVHFQKLDFPAVTICNINPYKYSAVSDLLTDLDSETKQALLSLYGVKDVLDSTPRKRREAGSMRSTWEGTPPRFLNLIPLLVFNENEKGKARDFFTGRKRKISGKIIHKASNVMHVHESKKLVGFQLCSNDTSDCATYTFSSGINAIQEWYKLHYMNIMAQVPLEKKINMSYSAEELLVTCFFDGMSCDARNFTLFHHPMYGNCYTFNNRENATILSTSMGGSEYGLQVILYINEDEYNPFLVSSTGAKVLVHQQNEYPFIEDVGTEIETAMSTSIGMHLTESFKLSEPYSQCTEDGSDVPVTNIYNAAYSLQICLYSCFQTKMVEKCGCAQYSQPLPPAANYCNYQQHPNWMYCYYQLYQAFVREELGCQSVCKQSCSFKEWTLTTSLAQWPSEASEKWLLNVLTWDQSQQINKKLNKTDLAKLLIFYKDLNQRSIMESPANSIEMLLSNFGGQLGLWMSCSVVCVIEIIEVFFIDFFSIIARRQWQKAKDWWARRRTPPSTETPSSQQGQDNPALDTDDDLPTFTSAMRLPPAPEAPVPGTPPPRYNTLRLDSAFSSQLTDTQLTNEF

SEQ ID NO: 7 - protein, 4 aa, Artificial
  Cleavage site on ENaC gamma by furin
RKRK

SEQ ID NO: 8 - protein, 26 aa, Rattus norvegicus
DLLGAFPHPLQRLRTPPPPYSGRTAR

SEQ ID NO: 9 - protein, 26 aa, Bos taurus
DLREPLPHPLQRLPVPAPPHAARGVR

SEQ ID NO: 10 - protein, 26 aa, Oryctolagus cuniculus
DVHPPLPHPLQRLRVPPPRLEARRAR

SEQ ID NO: 11 - protein, 43 aa, Rattus norvegicus
EAGSMPSTLEGTPPRFFKLIPLLVFNENEKGKARDFFTGRKRK

SEQ ID NO: 12 - protein, 43 aa, Canis familiaris
EAESWSSAWEGTRPKFLRLVPLMVFSQDETSQARDFLTGRKRK

SEQ ID NO: 13 - protein, 43 aa, Bos taurus
EAQSWSSVRKGTDPKFLNLAPLMAFEKGDTGKARDFFTGRKRK

SEQ ID NO: 14 - protein, 44 aa, Oryctolagus cuniculus
DTESWSPAWEGVRPKFLNLVPLLIFNRDEKGKARDFLSLGRKRK

SEQ ID NO: 15 - protein, 26 aa, Artificial
  Scrambled alpha peptide used as control experiments for Examples
PLTQPRPRLADHNPLSARRRPLPAGP

SEQ ID NO: 16 - protein, 26 aa, Artificial
  Substituted alpha peptide where all Proline are substituted with Alanine
DLRGALAHALQRLRTAAAANAARSAR

SEQ ID NO: 17 - protein, 26 aa, Artificial
  Substituted alpha peptide where all Arginines are substituted with Glutamic Acid
DLEGALPHPLQELETPPPPNPAESAE

SEQ ID NO: 18 - protein, 43 aa, Artificial
  Scrambled gamma peptide used as control experiments for Examples
FKGFVGKEALREILTFWLRFNNTEMDSKPLRTRANPPSKGGRE

SEQ ID NO: 19 - protein, 4 aa, Artificial
  Cleavage site of ENaC gamma by furin was mutated to this sequence
QQQQ

SEQ ID NO: 20 - protein, 43 aa, Artificial Sequence
  Artificial ENaC alpha sequence in which X30 is omitted
  MISC_FEATURE (1)..(40): Any X can be any amino acid
EAXSXXSXXEGXXPRFXXXIPLLXFXXXEKGKARDFFTGXKRK